(19) United States
(12) Patent Application Publication
(10) Pub. No.: US 2023/0002346 A1
(43) Pub. Date: Jan. 5, 2023

(54) NEW COMPOUNDS AND METHODS

(71) Applicant: BenevolentAl Bio Limited, London (GB)

(72) Inventors: Michael J. Rawling, London (GB); Edward I. Balmond, London (GB); Callum Finn, London (GB); Márton Vass, London (GB); Mark David Rackham, London (GB); Edward D. Savory, London (GB); Rebecca Paul, London (GB)

(73) Assignee: BenevolentAl Bio Limited, London (GB)

(21) Appl. No.: 17/642,060

(22) PCT Filed: Sep. 11, 2020
(86) PCT No.: PCT/GB2020/052203

§ 371 Date: Mar. 10, 2022

(30) Foreign Application Priority Data
Sep. 11, 2019 (GB) 1913110.1

(51) Int. Cl. C07D 401/04; C07D 213/82; C07D 405/04; C07D 417/04; C07D 413/04; C07D 401/10; C07D 491/056; C07D 471/04; C07D 491/048

(52) U.S. Cl. CPC C07D 401/04; C07D 213/82; C07D 405/04; C07D 417/04; C07D 413/04; C07D 401/10; C07D 491/056; C07D 471/04; C07D 491/048

(57) ABSTRACT

The present invention relates to compounds of Formula (I) or a pharmaceutically acceptable salt, solvate, hydrate, tautomer, optical isomer, N-oxide, and/or prodrug thereof. The present invention also relates to pharmaceutical compositions comprising the compounds of the invention, and to their use in the treatment or prevention of medical conditions in which inhibition of c-Abl is beneficial. (I)

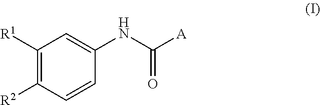

### FIELD OF THE INVENTION

The present invention relates to compounds of Formula (I) which are inhibitors of c-Abl. The invention also relates to pharmaceutical compositions comprising those compounds, and to their use in the treatment or prevention of medical conditions in which inhibition of c-Abl is beneficial. Such medical conditions include neurodegenerative diseases and cancer.

### BACKGROUND

ABL1 (Abelson Murine Leukaemia Viral Oncogene Homolog 1) is a protein that exhibits tyrosine kinase enzymatic activity and is associated with various cell functions. In humans, this protein is encoded by the ABL1 gene located on chromosome 9. The version of the ABL1 gene found within the mammalian genome is denoted c-Abl.

Philadelphia chromosome is a genetic abnormality in chromosome 22 formed by the t(9,22) reciprocal chromosome translocation, resulting in a fusion gene denoted BCR-ABL1. This fusion gene contains the ABL1 gene from chromosome 9 and part of the BCR gene. The tyrosine kinase activity of the ABL1 protein is normally tightly regulated, however, the BCR domains in the fusion gene result in constitutive activation of the ABL1 kinase. However, the binding domains of BCR-ABL and c-Abl are identical.

Activation of c-Abl has been implicated in various diseases, notably cancer. For instance, the presence of the BCR-ABL mutation is strongly linked to chronic myeloid leukaemia (CML). It is also found in some instances of acute lymphoblastic leukaemia (ALL). Nilotinib and Ponatinib are both orthosteric c-Abl inhibitors which bind in the ATP-site of c-Abl and have been used in the treatment of chronic myeloid leukaemia (CML) and acute lymphoblastic leukaemia (ALL).

Asciminib (ABL001) is an allosteric c-Abl inhibitor which binds in the myristate pocket of c-Abl. Asciminib is currently in clinical trials for the treatment of CML and Philadelphia chromosome-positive ALL, either as a standalone therapy or in combination with orthosteric tyrosine kinase inhibitors of c-Abl, such as nilotinib, ponatinib, dasatinib, and bosutinib.

The range of leukaemias that may be treated by c-Abl inhibition include chronic myeloid leukaemia (CML), acute lymphoblastic leukaemia (ALL), acute myelogenous leukaemia (AML), mixed-phenotype acute leukaemia (MPAL), and central nervous system (CNS) metastases thereof.

Activation of c-Abl has also been implicated in neurodegenerative diseases. Neurodegenerative diseases may be characterised by progressive degeneration and ultimate death of neurons. Particular neurodegenerative diseases include amyotrophic lateral sclerosis (ALS) and Parkinson's disease (PD).

ALS is a fatal neurodegenerative disease caused by the progressive degeneration of motor neurons. It has been reported that c-Abl signalling activation contributes to neuronal apoptosis and that c-Abl inhibitors can prevent motor neuron death [Rojas et al. Frontiers in Cellular Neuroscience, 2015, 9, 203; Imamura et al. Science Translational Medicine, 2017].

Parkinson's disease (PD) is a progressive neurodegenerative disorder caused by a selective loss of dopaminergic neurons in the substantia nigra pars compacta. It has been reported that c-Abl is activated in the brain of patients with PD and that c-Abl inhibition can protect against dopamine neuronal loss [Pagan et al. Pharmacology Research & Perspectives, 2019; Karuppagounder et al. Scientific Reports, 2014, 4, 4874].

Activation of c-Abl has also been implicated in a wide range of other diseases including, but not limited to, prion diseases, viral infections, diabetes, inflammatory diseases such as pulmonary fibrosis, and skeletal or muscular dystrophies.

Viral infections can be mediated by ABL1 kinase activity, as in the case of pox-viruses and the Ebola virus. Gleevec® and Tasigna® have been shown to stop the release of Ebola viral particles from infected cells, in vitro (see for instance WO 2007/002441; Mayra et al. Productive Replication of Ebola Virus Is Regulated by the ABL1 Tyrosine Kinase Science translational medicine 2012, 4, 123ra24). Inhibition of the ABL kinase can therefore be expected to reduce the pathogen's ability to replicate.

In prion disease models, Gleevec® showed beneficial effects. It delayed prion neuroinvasion by inhibiting prion propagation from the periphery to the CNS (Yun et al. The tyrosine kinase inhibitor imatinib mesylate delays prion neuroinvasion by inhibiting prion propagation in the periphery J Neurovirol. 2007, 13, 328-37). Gleevec® and ABL deficiency induced cellular clearance of PrPSc in prion-infected cells (Ertmer et al. The tyrosine kinase inhibitor STI571 induces cellular clearance of PrPSc in prion-infected cells J. Biol. Chem. 2004 279, 41918-27). Therefore, ABL1 inhibitors represent a valid therapeutic approach for the treatment of prion diseases, such as Creutzfeldt-Jacob disease (CJD).

X-linked recessive Emery-Dreifuss muscular dystrophy is caused by mutations of emerin, a nuclear-membrane protein with roles in nuclear architecture, gene regulation and signalling. A study has shown that emerin is tyrosine-phosphorylated directly by ABL1 in cell models, and that the phosphorylation status of emerin changes emerin binding to other proteins such as BAF. This, in turn, may explain the mislocalization of mutant emerin from nuclear to cytosolic compartments and consequently changes in downstream effector and signal integrator for signalling pathway(s) at the nuclear envelope (Tifft et al. Tyrosine phosphorylation of nuclear-membrane protein emerin by SRC, ABL1 and other kinases J. Cell Sci. 2009, 122, 3780-90). Changes in emerin-lamin interactions during both mitosis and interphase are of relevance for the pathology of muscular dystrophies. In addition, results from another study demonstrate that Gleevec® attenuates skeletal muscle dystrophy in mdx mice (Huang et al. Imatinib attenuates skeletal muscle dystrophy in mdx mice FASEB J. 2009, 23, 2539-48). Therefore, ABL1 inhibitors also represent therapeutic approaches for treatment of skeletal and muscular dystrophies.

Furthermore, ABL1 kinase plays a role in inflammation and oxidative stress, two mechanisms that are implicated in a variety of human diseases ranging from acute CNS diseases, such as stroke and traumatic brain or spinal cord injuries, chronic CNS diseases, such as Alzheimer's, Parkinson's, Huntington's and motoneuron diseases, to non-CNS inflammatory and autoimmune diseases, such as diabetes, pulmonary fibrosis.

For example, Gleevec® prevents fibrosis in different preclinical models of systemic sclerosis and induces regression of established fibrosis (Akhmetshina et al. Treatment with imatinib prevents fibrosis in different preclinical models of systemic sclerosis and induces regression of established fibrosis Arthritis Rheum. 2009, 60, 219-24) and it shows antifibrotic effects in bleomycin-induced pulmonary fibrosis in mice (Aono et al. Imatinib as a novel antifibrotic agent in bleomycin-induced pulmonary fibrosis in mice Am. J. Respir. Crit. Care Med. 2005, 171, 1279-85). Another study showed that both imatinib and nilotinib attenuated bleomycin-induced acute lung injury and pulmonary fibrosis in mice (Rhee et al. Effect of nilotinib on bleomycin-induced acute lung injury and pulmonary fibrosis in mice. Respiration 2011, 82, 273-87). Although in these studies the authors were focusing on the implication the mechanism related to PDGFRs, of interest, in the study by Rhee et al. (Respiration. 2011, 82, 273-87), nilotinib which is a more potent c-Abl inhibitor than imatinib showed superior therapeutic antifibrotic effects, thus supporting the therapeutic applicability of c-Abl inhibitors for treatment of human diseases with pulmonary inflammation. In another study, exposure of mice to hyperoxia increased ABL1 activation which is required for dynamin 2 phosphorylation and reactive oxygen species production and pulmonary leak (Singleton et al. Dynamin 2 and c-Abl are novel regulators of hyperoxia-mediated NADPH oxidase activation and reactive oxygen species production in caveolin-enriched microdomains of the endothelium J. Biol. Chem. 2009, 284, 34964-75).

It has also been reported that c-Abl inhibition may be a useful strategy for ameliorating local and system inflammation in severe acute pancreatitis [R Madhi et al, Journal of Leukocyte Biology, 2019, 106(2): 455-466. Further, BCR-ABL inhibitors have been used in the treatment of pulmonary arterial hypertension [D. Dumitrescu et al, European Respiratory Journal, 2011, 38: 218-220].

In view of the above there is an unmet need for new compounds that may be used in the treatment and prevention of medical conditions in which inhibition of c-Abl is beneficial, such as neurodegenerative diseases (i.e. ALS and PD) and cancer (especially leukaemias).

### DISCLOSURE OF THE INVENTION

Surprisingly, it has been found that compounds of Formula (I) inhibit c-Abl and therefore treat or prevent the above medical conditions. Without wishing to be bound by theory, it is believed that compounds of Formula (I) bind in the myristate pocket of c-Abl and therefore operate via an allosteric inhibitory mechanism.

Further, the compounds of Formula (I) have certain beneficial properties leading to increased potential for use as a drug compared to known compounds. This may be in terms of their efficacy, brain to plasma ratio, bioavailability, clearance, half-life, solubility, selectivity profiles, such as kinase selectivity, low hERG inhibitory activity, and/or other notable pharmacokinetic properties.

Consequently, the invention relates to a compound of Formula (I),

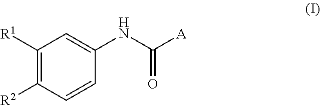

or a pharmaceutically acceptable salt, solvate, hydrate, geometrical isomer, tautomer, optical isomer, N-oxide thereof, and/or prodrug thereof, wherein

R¹ is selected from the group consisting of H and halo;

R² is selected from the group consisting of —OCF₂Cl, —OCF₃, —SCF₃, —SCF₂Cl, —CF₂CF₃, —CF₂CF₂Cl, —OCF₂CF₃, —SF₅, OF₂CH₃, —SOCF₃, —SO₂CF₃, —OCF₂CF₂H, and —SCF₂H;

A is

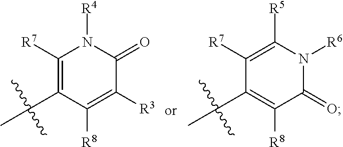

R³, R⁴, R⁵, and R⁶ are independently selected from the group consisting of:

(i) H, halo, —OH, —C(O)NR^{d}R^{e}, —NR^{a}R^{b}, cyano, —C(O)OR^{c}, and —C(O)R^{c};

(ii) C₁-C₇ alkyl, C₂-C₆ alkenyl, C₂-C₆ alkynyl, C₁-C₆ alkoxy, optionally substituted with one or more substituents independently selected from the group consisting of —NR^{a}R^{b}, cyano, —OR^{c}, halo, oxo, 6- to 10-membered aryl, 5- to 10-membered heteroaryl, and 4- to 10-membered heterocycle, optionally wherein the 5- to 10-membered heteroaryl and 4- to 10-membered heterocycle are independently substituted with one or more substituents selected from halo and C₁-C₇ alkyl, wherein the C₁-C₇ alkyl is optionally substituted with one or more halo atoms;

(iii) 6- to 10-membered aryl and 5- to 10 membered heteroaryl, optionally substituted with one or more substituents independently selected from the group consisting of halo, —C(O)NR^{d}R^{e}, —NR^{d}R^{e}, —OH, oxo, cyano, —C(O)OR^{c}, and —C(O)R^{c}, C₁-C₇ alkyl, C₂-C₆ alkenyl, C₂-C₆ alkynyl, C₁-C₆ alkoxy, (C₁-C₃ alkyl)-O—(C₁-C₃ alkyl), and 4- to 10-membered heterocycle, wherein the 4- to 10-membered heterocycle is optionally substituted with an oxo group, wherein the alkyl, alkenyl, and alkynyl groups are each optionally independently substituted with one or more halo atoms; and

(iv) 4- to 10-membered heterocycle, optionally substituted with one or more substituents independently selected from the group consisting of halo, oxo, —C(O)NR^{d}R^{e}, —NR^{d}R^{e}, cyano, —C(O)OR^{c}, and —C(O)R^{c}, C₁-C₇ alkyl, C₂-C₆ alkenyl, C₂-C₆ alkynyl, C₁-C₆ alkoxy, (C₁-C₃ alkyl)-O—(C₁-C₃ alkyl), 6- to 10-membered aryl, 5- to 10-membered heteroaryl, and 4- to 10-membered heterocycle, wherein the alkyl, alkenyl, and alkynyl groups are each optionally independently substituted with one or more halo atoms;

each R⁷ and R⁸ is independently selected from the group consisting of H and halo;

each R^{a}, R^{b}, and R^{c} is independently selected from H and C₁-C₇ alkyl, wherein the C₁-C₇ alkyl is optionally substituted with one or more halo atoms; and

each R^{d} and R^{e} are independently selected from H and C₁-C₇ alkyl, wherein the C₁-C₇ alkyl is optionally substituted with one or more halo atoms, or R^{d} and R^{e} can be taken together with the nitrogen atom to which they are attached to form a 5- or 6-membered saturated, partially saturated, or unsaturated ring, wherein the ring contains one or more heteroatoms.

These compounds are compounds of the invention.

It is preferable that R¹ is selected from H, F, and Cl, and more preferably R¹ is H.

Each R⁷ and R⁸ is also preferably selected from H, F, and Cl, more preferably H and F, and most preferably H.

In a preferred feature of the invention, R¹ and each R⁷ and R⁸ are H.

In another preferred feature of the invention, R² is selected from —OCF₂Cl and —OCF₃.

Notwithstanding the above, the point of attachment on R⁴ and R⁶ to the remainder of the molecule is not a halo atom, and preferably not a heteroatom other than in the case of forming an N-oxide. As such, R⁴ and R⁶ are not halo, and R⁴ and R⁶ are preferably not —NR^{a}R^{b}, or C₁-C₆ alkoxy.

It may be particularly advantageous to include an aromatic or heteroaromatic substituent on group A in the meta-position relative to the amide linker (i.e. in positions R³, R⁴, or R⁵, as present in Formula (I)), as this may result in increased inhibition of c-Abl. As such, compounds of Formula (I) that are particularly preferred are those in which R⁵ and R³ and/or R⁴ are selected from substituents (iii) listed above. It is especially preferred that R⁵ or R⁴ is selected from substituents (iii).

It is highly preferred that R¹, R⁷, and R⁸ are H and R² is selected from —OCF₂C₁ and —OCF₃. In this case, the compounds of the invention are compounds of Formula (II),

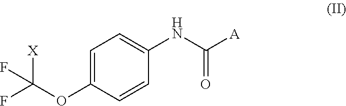

wherein X is F or C₁;

A is

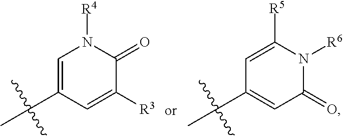

and

R³, R⁴, R⁵, and R⁶ are defined as above.

Compounds of Formula (II) in which X is CI are particularly preferred as these compounds may exhibit increased inhibition of c-Abl.

In a preferred feature of the invention, R³, R⁴, R⁵, and R⁶ are independently selected from the group consisting of:

(i) H and cyano;

(ii) C₁-C₆ alkyl and C₁-C₆ alkoxy, optionally substituted with one or more substituents independently selected from halo and 5- or 6-membered heterocycle;

(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and
(iv) phenyl optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;

(v) 5- to 10-membered heteroaryl optionally substituted with one or more substituents independently selected from the group consisting of halo, cyano, oxo, C₁-C₆ alkyl, C₁-C₆ alkoxy, and (C₁-C₃ alkyl)-O—(C₁-C₃ alkyl), wherein the alkyl groups are optionally substituted with one or more halo atoms.

In this preferred feature of the invention, compounds of Formula (II) that are particularly preferred are those in which R⁵ and R³ and/or R⁴ are selected from substituents (iv) and (v). It is especially preferred that R⁵ or R⁴ is selected from substituents (iv) and (v).

In one aspect of the invention, the compound of Formula (II) is a compound of Formula (IIa),

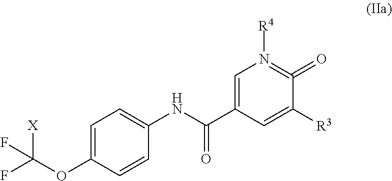

wherein R³ and R⁴ are independently selected from the group consisting of:

(i) H and cyano;

(ii) C₁-C₆ alkyl and C₁-C₆ alkoxy, optionally substituted with one or more substituents independently selected from halo and 5- or 6-membered heterocycle;

(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and

(iv) phenyl, optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;

(v) 5- to 10-membered heteroaryl optionally substituted with one or more substituents independently selected from the group consisting of halo, cyano, oxo, C₁-C₆ alkyl, C₁-C₆ alkoxy, and (C₁-C₃ alkyl)-O—(C₁-C₃ alkyl), wherein the alkyl groups are optionally substituted with one or more halo atoms.

Compounds of Formula (IIa) that are particularly preferred are those in which R³ and/or R⁴ is selected from substituents (iv) and (v). It is especially preferred that R⁴ is selected from substituents (iv) and (v).

It is preferred that R³ is selected from the group consisting of:

(i) H, C₁-C₆ alkoxy, and cyano;

(ii) C₁-C₆ alkyl, optionally substituted with one or more halo atoms;

(iii) phenyl and 5- or 6-membered heteroaryl, optionally substituted with one or more substituents independently selected from the group consisting of halo and C₁-C₆ alkyl, wherein the C₁-C₆ alkyl is optionally substituted with one or more halo atoms; and

(iv) 5- or 6-membered heterocycle, optionally substituted with one or more substituents independently selected from the group consisting of halo and C₁-C₆ alkyl, wherein the C₁-C₆ alkyl is optionally substituted with one or more halo atoms.

Compounds of Formula (IIa) that are particularly preferred are those in which R³ is selected from substituents (iii), i.e. optionally substituted phenyl and 5- or 6-membered heteroaryl.

Examples of particularly preferred 5- or 6-membered heteroaryls as R³ include

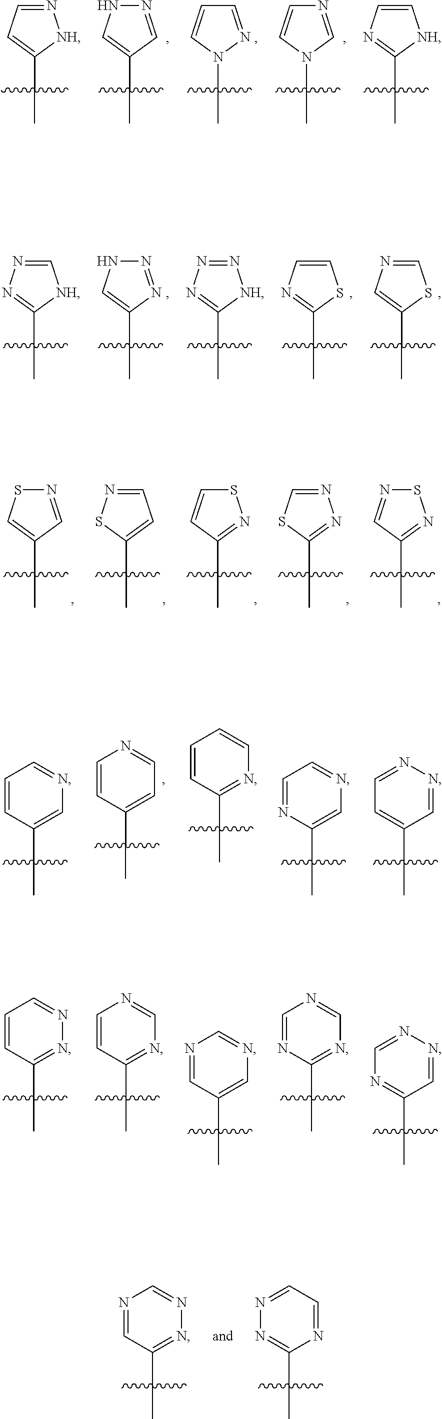

or tautomers thereof, each of which may be optionally substituted as outlined above. More preferably they include:

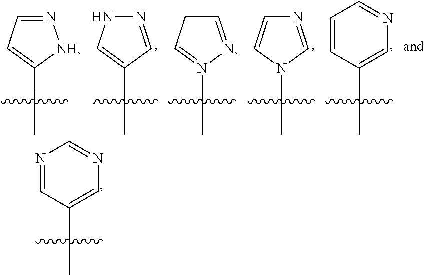

or tautomers thereof, each of which may be optionally substituted as outlined above.

Further, it is preferred that R⁴ is selected from the group consisting of:

(i) H;

(ii) C₁-C₆ alkyl, wherein the C₁-C₆ alkyl is optionally substituted with one or more substituents selected from halo and 5- or 6-membered heterocycle;

(iii) phenyl, optionally substituted with one or more substituents independently selected from halo, cyano, C₁-C₆ alkyl, C₁-C₆ alkoxy, and 4-membered heterocycle, wherein the 4-membered heterocycle is optionally substituted with an oxo group, wherein the C₁-C₆ alkyl is optionally substituted with one or more halo atoms;

(iv) 5- or 6-membered heteroaryl, optionally substituted with one or more substituents independently selected from halo, cyano, C₁-C₆ alkyl, C₁-C₆ alkoxy, and (C₁-C₃ alkyl)-O—(C₁-C₃ alkyl), wherein the alkyl groups are optionally substituted with one or more halo atoms;

(v) 5- or 6-membered heterocycle, optionally substituted with one or more substituents independently selected from the group consisting of halo, and C₁-C₆ alkyl, wherein the C₁-C₆ alkyl is optionally substituted with one or more halo atoms;

(vi) group B

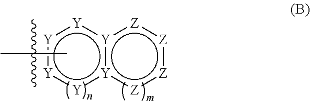

wherein each Y and Z is independently selected from C, S, O, and N, at least one Y or Z is S, O or N,
each Y and Z is optionally independently substituted with halo, or C₁-C₆ alkyl, wherein the C₁-C₆ alkyl is optionally substituted with one or more halo atoms,
n is 0 or 1,
m is 0 or 1;

(vii) group C

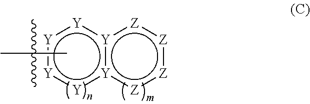

wherein each Y and Z is independently selected from C, S, O, and N, at least one Y or Z is S, O or N,
each Y and Z is optionally independently substituted with halo or C₁-C₆ alkyl, wherein the C₁-C₆ alkyl is optionally substituted with one or more halo atoms,
n is 0 or 1,
m is 0 or 1; and

(viii) group D

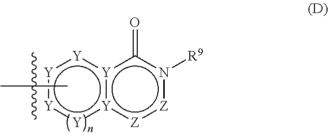

wherein each Y and Z is independently selected from C, S, O, and N, at least one Z is C,
each Y and Z is optionally independently substituted with halo or C₁-C₆ alkyl, wherein the C₁-C₆ alkyl is optionally substituted with one or more halo atoms, R⁹ is selected from halo and C₁-C₆ alkyl, wherein the C₁-C₆ alkyl is optionally substituted with one or more halo atoms,
n is 0 or 1;

Compounds of Formula (IIa) that are particularly preferred are those in which R⁴ is selected from substituents (iii), (iv), (vi), (vii), and (viii).

Exemplary 5- or 6-membered heteroaryls as R⁴ include:

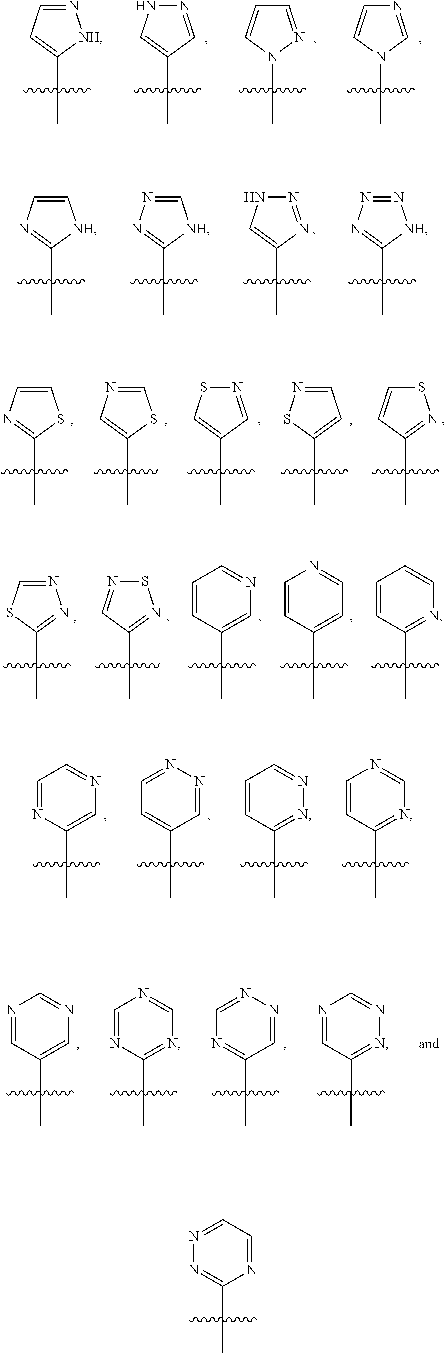

or tautomers thereof, each of which may be optionally substituted as outlined above. More preferably they include:

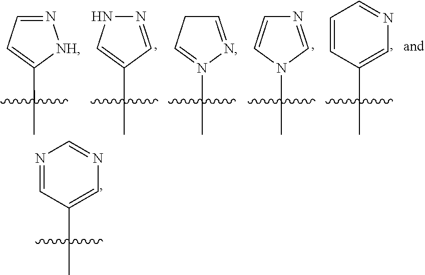

or tautomers thereof, each of which may be optionally substituted as outlined above.

Particularly preferred examples of groups B, C, and D as R⁴ include:

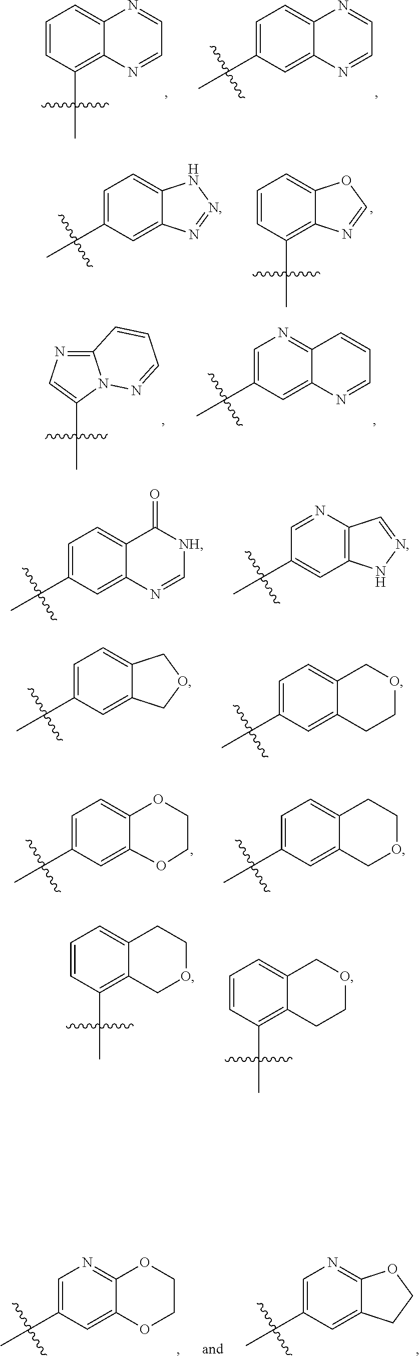

or tautomers thereof, each of which may be optionally substituted as outlined above.

In another aspect of the invention, the compound of Formula (II) is a compound of Formula (IIb),

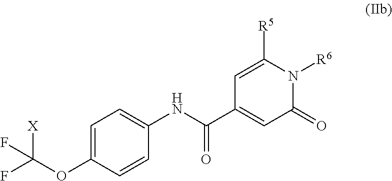

wherein R⁵ and R⁶ are independently selected from the group consisting of:

(i) H and cyano;

(ii) C₁-C₆ alkyl and C₁-C₆ alkoxy, optionally substituted with one or more substituents independently selected from halo and 5- or 6-membered heterocycle; and

(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and

(iv) phenyl, optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;

(v) 5- to 10-membered heteroaryl optionally substituted with one or more substituents independently selected from the group consisting of halo, cyano, oxo, C₁-C₆ alkyl, C₁-C₆ alkoxy, (C₁-C₃ alkyl)-O—(C₁-C₃ alkyl), wherein the alkyl groups are optionally substituted with one or more halo atoms;

Compounds of Formula (IIb) that are particularly preferred are those in which R⁵ is selected from substituents (iv) and (v).

It is preferred that R⁵ is a 5- or 6-membered heteroaryl group, optionally substituted with one or more substituents independently selected from the group consisting of halo and C₁-C₆ alkyl, wherein the C₁-C₆ alkyl is optionally substituted with one or more halo atoms.

Exemplary 5- or 6-membered heteroaryls as R⁵ include:

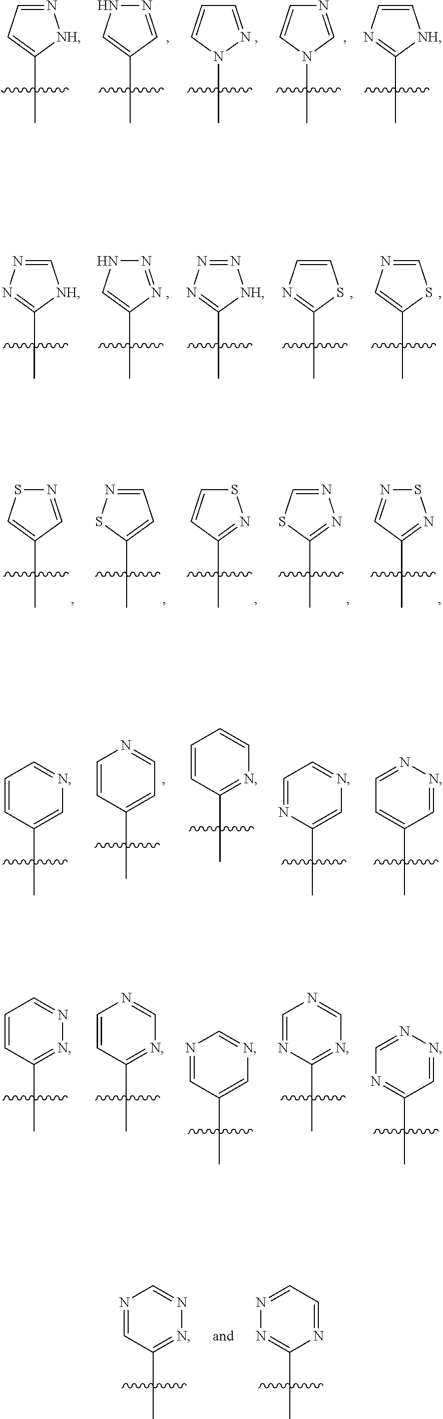

or tautomers thereof, each of which may be optionally substituted as outlined above. More preferably they include:

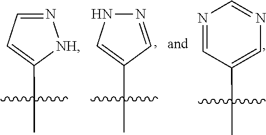

or tautomers thereof, each of which may be optionally substituted as outlined above.

Further, it is preferred that R⁶ is selected from the group consisting of H and C₁-C₆ alkyl, wherein the C₁-C₆ alkyl is optionally substituted with one or more halo atoms.

Particular compounds of the invention are those listed below.
N-[4-[Chloro(difluoro)methoxy]phenyl]-1-(3-ethoxy-1-methyl-pyrazol-4-yl)-6-oxo-pyridine-3-carboxamide;
1-(2-Morpholinoethyl)-6-oxo-N-[4-(trifluoromethoxy)phenyl]pyridine-3-carboxamide;
N-[4-(Chlorodifluoromethoxy)phenyl]-1-(1-methyl-1H-pyrazol-3-yl)-6-oxo-1,6-dihydropyridine-3-carboxamide;
N-[4-[Chloro(difluoro)methoxy]phenyl]-6-oxo-1-[(3S)-tetrahydrofuran-3-yl]pyridine-3-carboxamide;
N-[4-[Chloro(difluoro)methoxy]phenyl]-6-oxo-1-(1H-pyrazol-5-yl)pyridine-3-carboxamide;
N-[4-[Chloro(difluoro)methoxy]phenyl]-6-oxo-1-[(3R)-tetrahydrofuran-3-yl]pyridine-3-carboxamide;
N-[4-(Chlorodifluoromethoxy)phenyl]-1-[1-(difluoromethyl)-1H-pyrazol-3-yl]-6-oxo-1,6-dihydropyridine-3-carboxamide;
N-[4-[Chloro(difluoro)methoxy]phenyl]-6-oxo-1-thiazol-2-yl-pyridine-3-carboxamide;
N-[4-[Chloro(difluoro)methoxy]phenyl]-1-(2-methyl-3-pyridyl)-6-oxo-pyridine-3-carboxamide;
N-[4-[Chloro(difluoro)methoxy]phenyl]-1-(2,4-dimethoxypyrimidin-5-yl)-6-oxo-pyridine-3-carboxamide;
N-[4-[Chloro(difluoro)methoxy]phenyl]-6-oxo-1-thiazol-5-yl-pyridine-3-carboxamide;
N-[4-[Chloro(difluoro)methoxy]phenyl]-1-(2-methoxypyrimidin-5-yl)-6-oxo-pyridine-3-carboxamide;
N-[4-[Chloro(difluoro)methoxy]phenyl]-1-(6-methoxy-3-pyridyl)-6-oxo-pyridine-3-carboxamide;
N-[4-[Chloro(difluoro)methoxy]phenyl]-1-(1-isopropylpyrazol-4-yl)-6-oxo-pyridine-3-carboxamide;
N-[4-[Chloro(difluoro)methoxy]phenyl]-1-(1-cyclopropylpyrazol-4-yl)-6-oxo-pyridine-3-carboxamide;
N-[4-[Chloro(difluoro)methoxy]phenyl]-1-[(3R)-1-methylpyrrolidin-3-yl]-6-oxo-pyridine-3-carboxamide;
N-[4-[Chloro(difluoro)methoxy]phenyl]-1-[(3S)-1-methylpyrrolidin-3-yl]-6-oxo-pyridine-3-carboxamide;
N-[4-[Chloro(difluoro)methoxy]phenyl]-1-(1,5-dimethylpyrazol-4-yl)-6-oxo-pyridine-3-carboxamide;
N-[4-[Chloro(difluoro)methoxy]phenyl]-1-(3-cyclopropyl-1-methyl-pyrazol-4-yl)-6-oxo-pyridine-3-carboxamide;
N-[4-(Chlorodifluoromethoxy)phenyl]-1-(3-cyanophenyl)-6-oxo-1,6-dihydropyridine-3-carboxamide;
N-[4-[Chloro(difluoro)methoxy]phenyl]-1-[1-(cyclopropylmethyl)pyrazol-4-yl]-6-oxo-pyridine-3-carboxamide;
N-[4-[Chloro(difluoro)methoxy]phenyl]-1-[1-(2-methoxyethyl)pyrazol-4-yl]-6-oxo-pyridine-3-carboxamide; 1-(1-tert-Butylpyrazol-4-yl)-N-[4-[chloro(difluoro)methoxy]phenyl]-6-oxo-pyridine-3-carboxamide;
N-[4-(Chlorodifluoromethoxy)phenyl]-1-(3,4-difluorophenyl)-6-oxo-1,6-dihydropyridine-3-carboxamide;
N-[4-(Chlorodifluoromethoxy)phenyl]-1-(1-methyl-1H-pyrazol-5-yl)-6-oxo-1,6-dihydropyridine-3-carboxamide;
1-Methyl-6-oxo-N-[4-(trifluoromethoxy)phenyl]pyridine-3-carboxamide
N-[4-(Chlorodifluoromethoxy)phenyl]-1-[1-(difluoromethyl)-1H-pyrazol-4-yl]-6-oxo-1,6-dihydropyridine-3-carboxamide;
N-[4-(Chlorodifluoromethoxy)phenyl]-6-oxo-1-(quinoxalin-5-yl)-1,6-dihydropyridine-3-carboxamide;
N-[4-(Chlorodifluoromethoxy)phenyl]-1-(4-methoxyphenyl)-6-oxo-1,6-dihydropyridine-3-carboxamide;
1-(5-Chloro-1-methyl-1H-pyrazol-4-yl)-N-[4-(chlorodifluoromethoxy)phenyl]-6-oxo-1,6-dihydropyridine-3-carboxamide;
N-[4-(Chlorodifluoromethoxy)phenyl]-1-(2,3-difluorophenyl)-6-oxo-1,6-dihydropyridine-3-carboxamide;
N-[4-(Chlorodifluoromethoxy)phenyl]-1-(2,3-dimethoxyphenyl)-6-oxo-1,6-dihydropyridine-3-carboxamide;
N-[4-(Chlorodifluoromethoxy)phenyl]-1-(3,4-dihydro-1H-2-benzopyran-6-yl)-6-oxo-1,6-dihydropyridine-3-carboxamide;
N-[4-(Chlorodifluoromethoxy)phenyl]-1-(1,3-dihydro-2-benzofuran-5-yl)-6-oxo-1,6-dihydropyridine-3-carboxamide;
N-[4-(Chlorodifluoromethoxy)phenyl]-1-(3-methoxyphenyl)-6-oxo-1,6-dihydropyridine-3-carboxamide;
N-[4-(Chlorodifluoromethoxy)phenyl]-1-(2,3-dihydro-1,4-benzodioxin-6-yl)-6-oxo-1,6-dihydropyridine-3-carboxamide;
N-[4-(Chlorodifluoromethoxy)phenyl]-1-(2-fluorophenyl)-6-oxo-1,6-dihydropyridine-3-carboxamide;
N-[4-(Chlorodifluoromethoxy)phenyl]-6-oxo-1-(quinoxalin-6-yl)-1,6-dihydropyridine-3-carboxamide;
N-[4-(Chlorodifluoromethoxy)phenyl]-1-(3,4-dihydro-1H-2-benzopyran-7-yl)-6-oxo-1,6-dihydropyridine-3-carboxamide;
N-[4-(Chlorodifluoromethoxy)phenyl]-1-(3,4-dihydro-1H-2-benzopyran-8-yl)-6-oxo-1,6-dihydropyridine-3-carboxamide;
N-[4-(Chlorodifluoromethoxy)phenyl]-1-(3,4-dihydro-1H-2-benzopyran-5-yl)-6-oxo-1,6-dihydropyridine-3-carboxamide;
N-[4-(Chlorodifluoromethoxy)phenyl]-1-(4-fluorophenyl)-6-oxo-1,6-dihydropyridine-3-carboxamide;
N-[4-(Chlorodifluoromethoxy)phenyl]-1-(3,4-dimethoxyphenyl)-6-oxo-1,6-dihydropyridine-3-carboxamide;
N-[4-(Chlorodifluoromethoxy)phenyl]-1-(1-methyl-1H-1,2,3-benzotriazol-5-yl)-6-oxo-1,6-dihydropyridine-3-carboxamide;
1-(3-Chloro-1-methyl-1H-pyrazol-4-yl)-N-[4-(chlorodifluoromethoxy)phenyl]-6-oxo-1,6-dihydropyridine-3-carboxamide;
N-[4-(Chlorodifluoromethoxy)phenyl]-1-(3,5-difluorophenyl)-6-oxo-1,6-dihydropyridine-3-carboxamide;
N-[4-(Chlorodifluoromethoxy)phenyl]-1-(3-fluorophenyl)-6-oxo-1,6-dihydropyridine-3-carboxamide;
1-(1,3-Benzoxazol-4-yl)-N-[4-(chlorodifluoromethoxy)phenyl]-6-oxo-1,6-dihydropyridine-3-carboxamide;
N-[4-[Chloro(difluoro)methoxy]phenyl]-6-oxo-1-[4-(2-oxoazetidin-1-yl)phenyl]pyridine-3-carboxamide;
6-Oxo-1-(pyrimidin-5-yl)-N-[4-(trifluoromethoxy)phenyl]-1.6-dihydropyridine-3-carboxamide;
N-[4-(Chlorodifluoromethoxy)phenyl]-6-oxo-1-(pyrimidin-5-yl)-1,6-dihydropyridine-3-carboxamide;
N-[4-[Chloro(difluoro)methoxy]phenyl]-1-(1-methylpyrazol-4-yl)-6-oxo-pyridine-3-carboxamide;
N-[4-[Chloro(difluoro)methoxy]phenyl]-6-oxo-1-(3-pyridyl)pyridine-3-carboxamide;
N-[4-[Chloro(difluoro)methoxy]phenyl]-1-(5-fluoro-3-pyridyl)-6-oxo-pyridine-3-carboxamide;
N-[4-[Chloro(difluoro)methoxy]phenyl]-1-(5-cyano-3-pyridyl)-6-oxo-pyridine-3-carboxamide;
N-[4-[Chloro(difluoro)methoxy]phenyl]-6-oxo-1-phenyl-pyridine-3-carboxamide;
N-[4-[Chloro(difluoro)methoxy]phenyl]-1-(5-methyl-3-pyridyl)-6-oxo-pyridine-3-carboxamide;
N-[4-[Chloro(difluoro)methoxy]phenyl]-5-methoxy-6-oxo-1-pyrimidin-5-yl-pyridine-3-carboxamide;
N-[4-[Chloro(difluoro)methoxy]phenyl]-5-methyl-6-oxo-1-pyrimidin-5-yl-pyridine-3-carboxamide;
N-[4-(Chlorodifluoromethoxy)phenyl]-5-(morpholin-4-yl)-6-oxo-1-(pyrimidin-5-yl)-1,6-dihydropyridine-3-carboxamide;
N-[4-[Chloro(difluoro)methoxy]phenyl]-1-(5-methoxy-3-pyridyl)-6-oxo-pyridine-3-carboxamide;
N-[4-[Chloro(difluoro)methoxy]phenyl]-6-oxo-1-[5-(trifluoromethyl)-3-pyridyl]pyridine-3-carboxamide;
N-[4-[Chloro(difluoro)methoxy]phenyl]-1-(5-chloro-3-pyridyl)-6-oxo-pyridine-3-carboxamide;
N-[4-(Chlorodifluoromethoxy)phenyl]-1-{imidazo[1,2-b]pyridazin-3-yl}-6-oxo-1,6-dihydropyridine-3-carboxamide;
N-[4-(Chlorodifluoromethoxy)phenyl]-6′-methyl-2-oxo-2H-[1,3′-bipyridine]-5-carboxamide;
N-[4-(Chlorodifluoromethoxy)phenyl]-5′-(difluoromethyl)-2-oxo-2H-[1,3′-bipyridine]-5-carboxamide;
N-[4-(Chlorodifluoromethoxy)phenyl]-1-(1,5-naphthyridin-3-yl)-6-oxo-1,6-dihydropyridine-3-carboxamide;
N-[4-(Chlorodifluoromethoxy)phenyl]-1-(3-methyl-4-oxo-3,4-dihydroquinazolin-7-yl)-6-oxo-1,6-dihydropyridine-3-carboxamide;
N-[4-(Chlorodifluoromethoxy)phenyl]-1-{2H,3H-[1,4]dioxino[2,3-b]pyridin-7-yl}-6-oxo-1,6-dihydropyridine-3-carboxamide;
N-[4-(Chlorodifluoromethoxy)phenyl]-1-{1-methyl-1H-pyrazolo[4,3-b]pyridin-6-yl}-6-oxo-1,6-dihydropyridine-3-carboxamide;
N-[4-(Chlorodifluoromethoxy)phenyl]-1-{2-methyl-2H-pyrazolo[4,3-b]pyridin-6-yl}-6-oxo-1,6-dihydropyridine-3-carboxamide;
6′-Chloro-N-[4-(chlorodifluoromethoxy)phenyl]-2-oxo-2H-[1,3′-bipyridine]-5-carboxamide;
N-[4-(Chlorodifluoromethoxy)phenyl]-1-{2H,3H-furo[2,3-b]pyridin-5-yl}-6-oxo-1,6-dihydropyridine-3-carboxamide;
2′-Chloro-N-[4-(chlorodifluoromethoxy)phenyl]-2-oxo-2H-[1,3′-bipyridine]-5-carboxamide;
N-[4-(Chlorodifluoromethoxy)phenyl]-2-oxo-6′-(trifluoromethyl)-2H-[1.3′-bipyridine]-5-carboxamide;
N-[4-(Chlorodifluoromethoxy)phenyl]-1-methyl-6-oxo-1,6-dihydropyridine-3-carboxamide;
N-[4-(Chlorodifluoromethoxy)phenyl]-1-(oxan-4-yl)-6-oxo-1,6-dihydropyridine-3-carboxamide;
5-{[4-(Chlorodifluoromethoxy)phenyl]carbamoyl}-2-oxo-2H-[1.3′-bipyridin]-1′-ium-1′-olate;
N-[4-(Chlorodifluoromethoxy)phenyl]-5-cyano-1-(1-methyl-1H-pyrazol-4-yl)-6-oxo-1.6-dihydropyridine-3-carboxamide;
N-[4-(Chlorodifluoromethoxy)phenyl]-6-oxo-5-(1H-pyrazol-3-yl)-1,6-dihydropyridine-3-carboxamide;
N-[4-[Chloro(difluoro)methoxy]phenyl]-1-methyl-6-oxo-5-(3-pyridyl)pyridine-3-carboxamide;
N-[4-(Chlorodifluoromethoxy)phenyl]-1-methyl-6-oxo-5-(pyrimidin-5-yl)-1,6-dihydropyridine-3-carboxamide;
N-[4-(Chlorodifluoromethoxy)phenyl]-1-methyl-5-(1-methyl-1H-pyrazol-5-yl)-6-oxo-1,6-dihydropyridine-3-carboxamide;
N-[4-(Chlorodifluoromethoxy)phenyl]-5-(1-ethyl-1H-pyrazol-3-yl)-1-methyl-6-oxo-1,6-dihydropyridine-3-carboxamide;
N-[4-(Chlorodifluoromethoxy)phenyl]-1-methyl-5-(1-methyl-1H-pyrazol-3-yl)-6-oxo-1,6-dihydropyridine-3-carboxamide;
N-[4-[Chloro(difluoro)methoxy]phenyl]-1-methyl-5-(1-methylpyrazol-4-yl)-6-oxo-pyridine-3-carboxamide;
N-[4-[Chloro(difluoro)methoxy]phenyl]-5-(5-fluoro-3-pyridyl)-1-methyl-6-oxo-pyridine-3-carboxamide;
N-[4-[Chloro(difluoro)methoxy]phenyl]-1-methyl-6-oxo-5-phenyl-pyridine-3-carboxamide;
N-[4-(Chlorodifluoromethoxy)phenyl]-1-(1-methyl-1H-pyrazol-4-yl)-2-oxo-1,2-dihydro-[3,3′-bipyridine]-5-carboxamide;
N-[4-[Chloro(difluoro)methoxy]phenyl]-1-methyl-6-oxo-5-pyrazol-1-yl-pyridine-3-carboxamide;
N-[4-[Chloro(difluoro)methoxy]phenyl]-5-imidazol-1-yl-1-methyl-6-oxo-pyridine-3-carboxamide;
N-[4-(Chlorodifluoromethoxy)phenyl]-2-oxo-6-(1H-pyrazol-5-yl)-1,2-dihydropyridine-4-carboxamide;
2-Oxo-6-(pyrimidin-5-yl)-N-[4-(trifluoromethoxy)phenyl]-1,2-dihydropyridine-4-carboxamide;
N-[4-(Chlorodifluoromethoxy)phenyl]-1-methyl-6-(1-methyl-1H-pyrazol-4-yl)-2-oxo-1,2-dihydropyridine-4-carboxamide;

The compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, N-oxides, and/or prodrug forms thereof. It is to be understood that the compounds of the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

Another object of the present invention relates to the compounds of the invention for use in therapy.

The compounds of the invention are useful as inhibitors of c-Abl. As such, they are useful in the treatment or prevention of medical conditions (conditions or diseases) in which inhibition of c-Abl is beneficial. There is therefore provided a method for the treatment or prevention of a disease or condition responsive to c-Abl inhibition comprising administering a therapeutically effective amount of a compound of the invention to a subject. Whilst the compounds of the invention may be suitable to prevent a range of diseases and conditions, it is preferable that they are used to treat said diseases and conditions. Therefore, it is preferred that the method is for the treatment of a disease or condition, and therefore the method comprises administering a therapeutically effective amount of a compound of the invention to a subject in need thereof.

The term “treatment” as used herein may include prophylaxis of the named disorder or condition, or amelioration or elimination of the disorder once it has been established. The term “prevention” refers to prophylaxis of the named disorder or condition.

The range of diseases and conditions treatable or preventable by c-Abl inhibition is well known. The compounds of the invention therefore may be used to treat or prevent this range of diseases or conditions. This includes neurodegenerative disorders, cancers, prion diseases, viral infections, diabetes, inflammatory diseases such as pulmonary fibrosis, acute pancreatitis (preferably severe acute pancreatitis), pulmonary arterial hypertension, or a skeletal or muscular dystrophy. Preferably, the disease is a neurodegenerative disorder or a cancer.

Treatable or preventable neurodegenerative disorders include, but are not limited to, Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, and spinocerebellar ataxia, fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

Of the treatable or preventable neurodegenerative disorders, most notable are amyotrophic lateral sclerosis (ALS) and Parkinson's disease. Most preferably the neurodegenerative disorder is ALS.

Treatable or preventable cancers include, but are not limited to, leukaemia.

Of the treatable or preventable cancers, most notable are chronic myeloid leukaemia (CML), acute lymphoblastic leukaemia (ALL), acute myelogenous leukaemia (AML), and mixed-phenotype acute leukaemia (MPAL), or any central nervous system (CNS) metastases thereof. Most preferably the cancer is CML or ALL.

The invention thus includes the use of the compounds of the invention in the manufacture of a medicament for the treatment or prevention of a disease or condition, such as the above-mentioned neurodegenerative disorders and cancers. The invention also relates to the compounds of the invention for use in the treatment of a disease or condition, such as the above-mentioned neurodegenerative disorders and cancers.

The compounds of the invention can be used either as a standalone therapy or in conjunction with other c-Abl inhibitors. Examples of c-Abl inhibitors that can be used in conjunction with compounds of the invention are nilotinib, ponatinib, dasatinib, bosutinib, and mixtures thereof.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g. screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. Preferably, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g. to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g. Marker levels) in the sample can be performed using any suitable technique known in the art, including, but not limited to, enzyme immunoassay, is ELISA, radiolabelling/assay techniques, blotting/chemiluminescence methods, real-time PCR, and the like.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g. tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Definitions

The term “N-oxide” denotes a compound containing the N⁺—O⁻ functional group, such as in the following example.

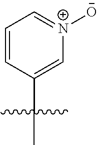 [SMILES: *c1ccc[n+]([O-])c1]

The term “heteroatom” means O, N, or S. It is preferable that a heteroatom is O or N.

“Optional” or “optionally” means that the subsequently described event or circumstance may, but need not, occur, and that the description includes instances where the event or circumstance occurs and instances in which it does not.

The term “C₁-C₇ alkyl” denotes a straight, branched or cyclic or partially cyclic alkyl group having from 1 to 7 carbon atoms, i.e. 1, 2, 3, 4, 5, 6, or 7 carbon atoms. For the “C₁-C₇ alkyl” group to comprise a cyclic portion it should be formed of 3 to 7 carbon atoms. For parts of the range “C₁-C₇ alkyl” all subgroups thereof are contemplated, such as C₁-C₆ alkyl, C₁-C₅ alkyl, C₁-C₄ alkyl, C₁-C₃ alkyl, C₁-C₂ alkyl, Ci alkyl, C₂-C₇ alkyl, C₂-C₆ alkyl, C₂-C₅ alkyl, C₂-C₄ alkyl, C₂-C₃ alkyl, C₂ alkyl, C₃-C₇ alkyl, C₃-C₆ alkyl, C₃-C₅ alkyl, C₃-C₄ alkyl, C₃ alkyl, C₄-C₇ alkyl, C₄-C₆ alkyl, C₄-C₅ alkyl, C₄ alkyl, C₅-C₇ alkyl, C₅-C₆ alkyl, C₅ alkyl, C₆-C₇ alkyl, C₆ alkyl, and C₇ alkyl. Examples of “C₁-C₇ alkyl” include methyl, ethyl, n-propyl, isopropyl, cyclopropyl, n-butyl, isobutyl, sec-butyl, t-butyl, cyclobutyl, cyclopropylmethyl, and straight, branched or cyclic or partially cyclic pentyl and hexyl etc.

When a term denotes a range, for instance “1 to 7 carbon atoms” in the definition of C₁-C₇ alkyl, each integer is considered to be disclosed, i.e. 1, 2, 3, 4, 5, 6 and 7.

The term “C₂-C₆ alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “C₂-C₆ alkenyl” all subgroups thereof are contemplated, such as C₂-C₆ alkenyl, C₂-C₄ alkenyl, C₂-C₃ alkenyl, C₂ alkenyl, C₃-C₆ alkenyl, C₃-C₆ alkenyl, C₃-C₄ alkenyl, C₃ alkenyl, C₄-C₆ alkenyl, C₄-C₆ alkenyl, C₄ alkenyl, C₅-C₆ alkenyl, C₅ alkenyl, and C₆ alkenyl. Examples of “C₂-C₆ alkenyl” include 2-propenyl, 2-butenyl, 3-butenyl, 2-methyl-2-propenyl, 2-hexenyl, 5-hexenyl, 2,3-dimethyl-2-butenyl.

The term “C₂-C₆ alkynyl” denotes a straight, branched or cyclic or partially cyclic alkyl group having at least one carbon-carbon triple bond, and having from 2 to 6 carbon atoms. The alkynyl group may comprise a ring formed of 3 to 6 carbon atoms. For parts of the range “C₂-C₆ alkynyl” all subgroups thereof are contemplated, such as C₂-C₆ alkynyl, C₂-C₄ alkynyl, C₂-C₃ alkynyl, C₂ alkynyl, C₃-C₆ alkynyl, C₃-C₆ alkynyl, C₃-C₄ alkynyl, C₃ alkynyl, C₄-C₆ alkynyl, C₄-C₆ alkynyl, C₄ alkynyl, C₅-C₆ alkynyl, C₅ alkynyl, and C₆ alkynyl. Examples of “C₂-C₆ alkynyl” include 2-propynyl, 2-butynyl, 3-butynyl, 2-pentynyl, 3-methyl-4-pentynyl, 2-hexynyl, 5-hexynyl etc.

The term “C₁-C₆ alkoxy” denotes —O—(C₁-C₆alkyl) in which a C₁-C₆ alkyl group is as defined above and is attached to the remainder of the compound through an oxygen atom. Examples of “C₁-C₆ alkoxy” include methoxy, ethoxy, n-propoxy, isopropoxy, n-butoxy, isobutoxy, sec-butoxy, t-butoxy and straight- and branched-chain pentoxy and hexoxy.

The term “halo” means a halogen atom, and unless otherwise stated is preferably, F, Cl, Br and I, more preferably F and Cl, and most preferably F.

The term “oxo” denotes a double bond to an oxygen atom (═O). This typically forms a ketone or aldehyde group, the former may form part of another functional group, such as a carboxylic acid, ester, or amide.

The term “6- to 10-membered aryl” denotes a stable aromatic monocyclic or fused bicyclic hydrocarbon ring system comprising 6 to 10 ring atoms. The term “6- to 10-membered aryl” includes fused bicyclic ring systems in which one ring is partially unsaturated or fully saturated, wherein the point of attachment to the remainder of the molecule is on the aromatic ring. Examples of “6- to 10-membered aryl” groups include phenyl, indenyl, naphthyl, naphthalene, 1,2,3,4-tetrahydronaphthyl, and indanyl.

The term “5- to 10-membered heteroaryl” denotes a stable aromatic monocyclic or fused bicyclic heteroaromatic ring system having 5 to 10 ring atoms in which 1 to 9 of the ring atoms are carbon and one or more of the ring atoms are selected from nitrogen, sulphur, and oxygen. Examples of “5- to 10-membered heteroaryl” include furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl, chromanyl, and tetrahydroquinoline. The term “5- to 10-membered heteroaryl” includes fused bicyclic ring systems in which one ring is partially unsaturated or fully saturated, wherein the point of attachment to the remainder of the molecule is on the aromatic ring, such as in the following examples:

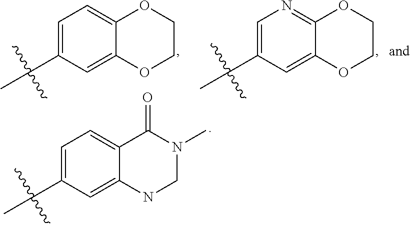

The term “5-membered heteroaryl” denotes a monocyclic “5- to 10-membered heteroaryl” having 5 ring atoms in which 1 to 4 of the ring atoms are carbon and one or more of the ring atoms are selected from nitrogen, sulfur, and oxygen. Examples of “5-membered heteroaryl” include pyrrolyl, and furyl.

The term “6-membered heteroaryl” denotes a monocyclic “5- to 10-membered heteroaryl” having 6 ring atoms in which 1 to 5 of the ring atoms are carbon and one or more of the ring atoms are selected from nitrogen, sulfur, and oxygen. Examples of “6-membered heteroaryl” include pyridinyl, and pyrimidinyl.

The term “4- to 10-membered heterocycle” denotes a non-aromatic monocyclic or fused bicyclic, fully saturated or partially unsaturated, ring system having 5 to 10 ring atoms in which 1 to 9 of the ring atoms are carbon and one or more of the ring atoms are selected from nitrogen, sulphur, and oxygen. When present, the sulfur atom may be in an oxidized form (i.e. the diradical of S═O or the diradical of O═S═O). The term “4- to 10-membered heterocycle” includes fused bicyclic ring systems in which one ring is aromatic, wherein the point of attachment to the reminder of the molecule is on the non-aromatic ring. Examples of “4- to 10-membered heterocycle” include azetidinyl, piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl, homopiperazinyl, and 5,6-dihydro-4H-1,3-oxazin-2-yl.

The term “5-membered heterocycle” denotes a monocyclic “4- to 10-membered heterocycle” in which the ring system has 5 ring atoms in which 1 to 4 of the ring atoms are carbon and one or more of the ring atoms are selected from nitrogen, sulfur, and oxygen. Examples of “5-membered heterocycle” include tetrahydrofuranyl, and pyrrolidinyl.

The term “6-membered heterocycle” denotes a monocyclic “4- to 10-membered heterocycle” wherein the ring system has 6 ring atoms in which 1 to 5 of the ring atoms are carbon and one or more of the ring atoms are selected from nitrogen, sulfur, and oxygen. Examples of “6-membered heterocycle” include piperidinyl, and morpholinyl.

“An effective amount” refers to an amount of a compound of the invention that confers a therapeutic effect on the treated subject. The therapeutic effect may be objective (i.e. measurable by some test or marker) or subjective (i.e. subject gives an indication of or feels an effect).

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Compounds of the invention may be disclosed by the name or chemical structure. Compounds herein were named using the OpenEye naming convention. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### Preparation of Compounds of the Invention

The compounds of formula (I) disclosed herein may be prepared by, or in analogy with, conventional methods. The preparation of Intermediates and compounds according to the Examples of the present invention may in particular be illuminated by the following Schemes. Definitions of variables in the structures in Schemes herein are commensurate with those of corresponding positions in the formulas delineated herein.

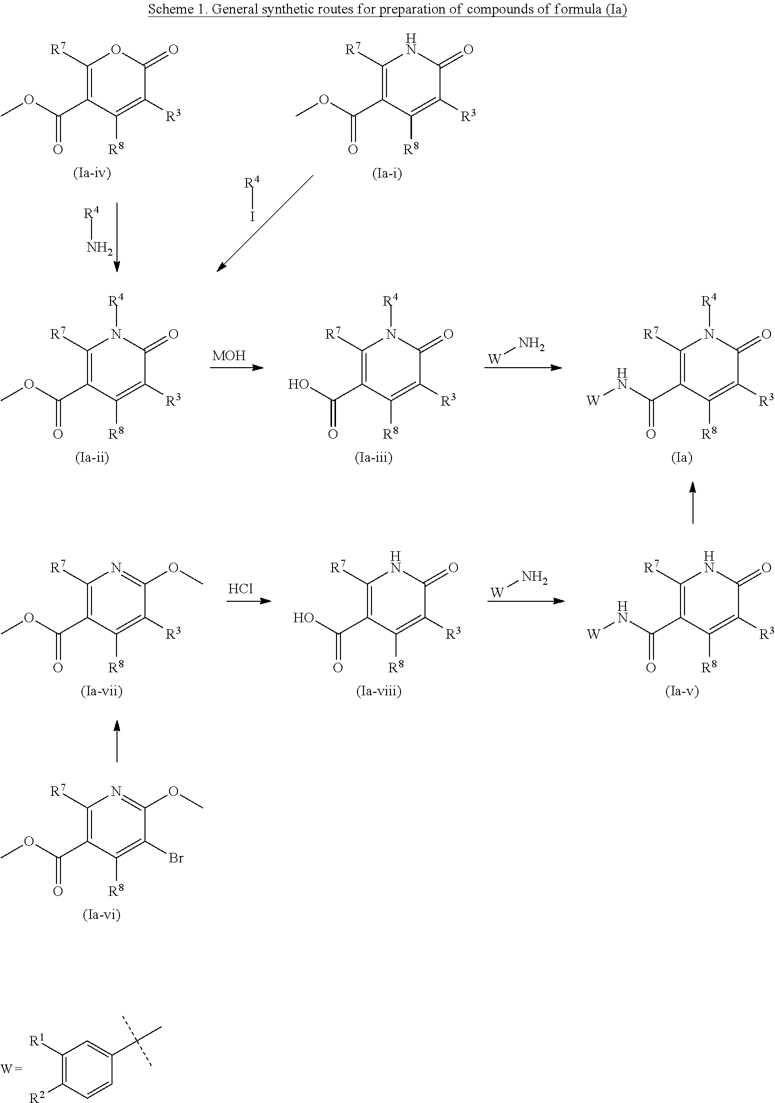

wherein R¹, R², R³, R⁴, R⁷ and R⁸ are as defined in formula (I) and M is Na or Li.

Compounds of general formula (I) where A is the pyridone regioisomer depicted in Scheme 1 (designated compounds of general formula (Ia)) can easily be prepared by a number of routes. For example, pyridones of general formula (Ia-i) can undergo N-alkylation with R⁴I alkyl iodides to compounds of general formula (Ia-ii), which can in turn undergo ester hydrolysis to give compounds of general formula (Ia-iii) and subsequent amide coupling with WNH₂ anilines to give compounds of general formula (Ia). Alternatively, compounds of general formula (Ia-ii) can also be prepared by ring-opening of coumalates of general formula (Ia-iv) with R⁴NH₂ amines followed by condensation-cyclisation. Alternatively, compounds of general formula (Ia) can be prepared from compounds of formula (Ia-v) by standard chemistry methodologies including alkylation, Chan-Lam or Ullmann reactions. Compounds of formula (Ia-v) can be prepared from 5-bromo-6-methoxy nicotinates of general formula (Ia-vi) using standard cross-coupling reactions such as Suzuki and Buchwald reactions, to give 6-methoxy nicotinates of general formula (Ia-vii), then treatment with acid to give compounds of general formula (Ia-viii) and subsequent amide coupling with WNH₂ anilines. If required, standard protecting group strategies can be employed to facilitate the syntheses outlined in Scheme 1.

Optionally, compounds of formula (Ia) can be converted into another compound of formula (Ia) in one or more synthetic steps.

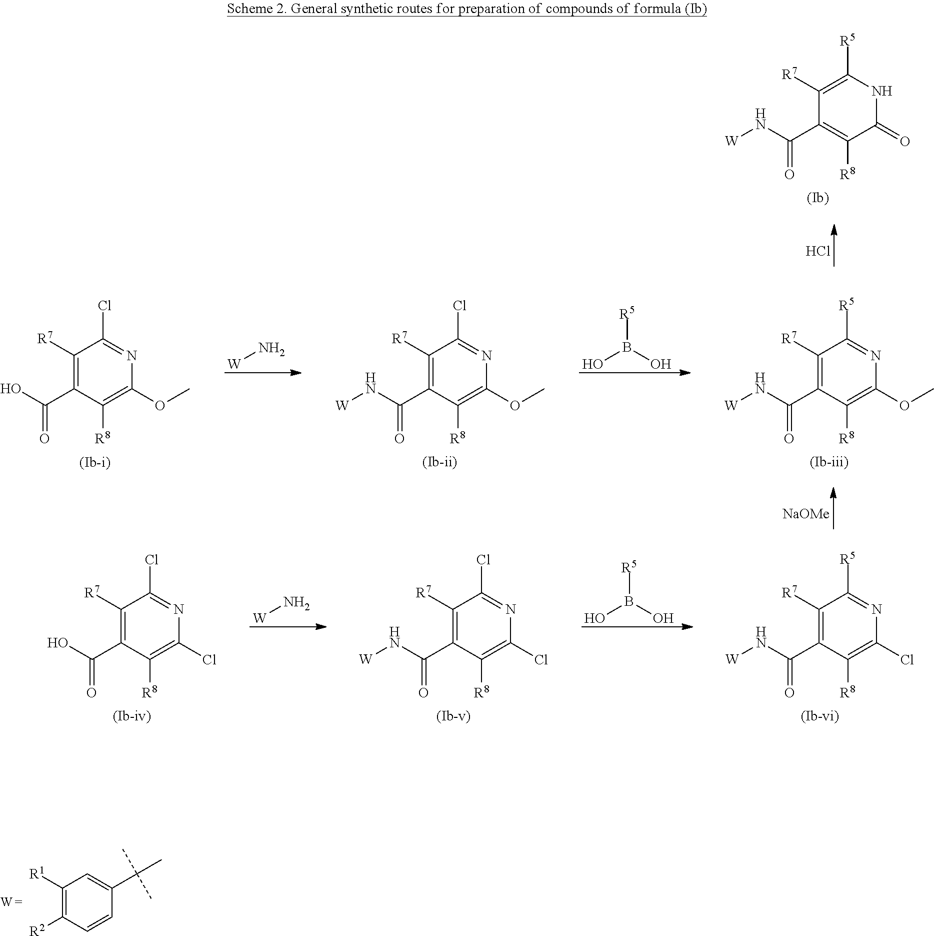

wherein R¹, R², R⁵, R⁷ and R⁸ are as defined in formula (I)

Compounds of general formula (I) where A is the pyridone regioisomer depicted in Scheme 2 and R⁶ is H (designated compounds of general formula (Ib)) can easily be prepared by a number of routes. For example, isonicotinic acids of general formula (Ib-i) can undergo amide formation with WNH₂ anilines to give isonicotinamides of general formula (Ib-ii), followed by Suzuki reaction to give compounds of general formula (Ib-iii). Compounds of general formula (Ib-iii) can then be treated with acids such as HCl, to give compounds of general formula (Ib). Alternatively, compounds of general formula (Ib-iii) can be prepared from isonicotinic acids of general formula (Ib-iv) by amide formation with WNH₂ anilines to give isonicotinamides of general formula (Ib-v), then Suzuki reaction to give compounds of general formula (Ib-vi) and subsequent treatment with sodium methoxide. If required, standard protecting group strategies can be employed to facilitate the syntheses outlined in Scheme 2.

Optionally, compounds of formula (Ib) can be converted into another compound of formula (Ib) in one or more synthetic steps.

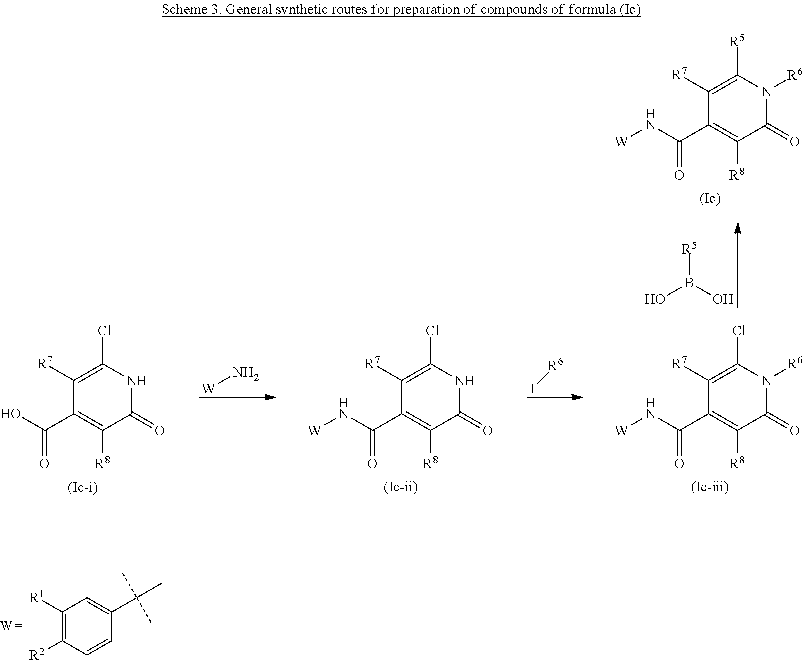

wherein R¹, R², R⁵, R⁶, R⁷ and R⁸ are as defined in formula (I)

Compounds of general formula (I) where A is the pyridone regioisomer depicted in Scheme 3 and R⁶ is not H (designated compounds of general formula (Ic)) can easily be prepared by standard means. For example, acids of general formula (Ic-i) can undergo amide formation with WNH₂ anilines to give compounds of general formula (Ic-ii), followed by N-alkylation with R⁶I alkyl iodides to give compounds of general formula (Ic-iii). Subsequent Suzuki reaction can give compounds of general formula (Ic). If required, standard protecting group strategies can be employed to facilitate the syntheses outlined in Scheme 3.

Optionally, compounds of formula (Ic) can be converted into another compound of formula (Ic) in one or more synthetic steps.

Appropriate reaction conditions for the individual reaction steps are known to a person skilled in the art. Particular reaction conditions for examples of the invention are also described in the experimental section. The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl(triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

The following abbreviations have been used:

| | |
|---|---|
| Ac | acetyl |
| AcOH | acetic acid |
| aq | aqueous |
| Ar | aryl |
| ATP | adenosine triphosphate |
| B:P | brain to plasma ratio |
| conc | concentrated |
| d | day(s) |
| DAD | diode array detector |
| dba | dibenzylideneacetone |
| DCM | dichloromethane |
| DIPEA | diisopropylethylamine |
| DMAP | 4-dimethylaminopyridine |
| DME | 1,2-dimethoxyethane |
| DMEDA | 1,2-dimethylethylenediamine |
| DMF | dimethylformamide |
| DMSO | dimethylsulfoxide |
| dppf | 1,1′-bis(diphenylphosphino)ferrocene |
| EDCI | 1-ethyl-3-(3-dimethylaminopropyl)carbodiimide |
| EDTA | ethylenediaminetetraacetic acid |
| ELSD | evaporative light scattering detector |
| equiv. | equivalent(s) |
| ES⁺ | electrospray ionization |
| Et₂O | diethylether |
| EtOAc | ethyl acetate |
| EtOH | ethanol |
| Ex. | example |
| GPCR | G-protein-coupled receptor(s) |
| h | hour(s) |
| HATU | 2-(7-aza-1H-benzotriazole-1-yl)-1,1,3,3-tetramethyluronium |
| | hexafluorophosphate |
| hERG | human ether-á-go-go related gene |
| Hex | hexane(s) |
| HPBC | 2-hydropropyl-beta-cyclodextrin |
| HPLC | high performance liquid chromatography |
| Int. | Intermediate |
| IPA | Isopropanol |
| i.v. | intravenous |
| LCMS | liquid chromatography mass spectrometry |
| LLoQ | lower limit of quantification |
| M | molar |
| mCPBA | meta-chloroperoxybenzoic acid |
| Me | methyl |
| MeCN | acetonitrile |
| MeOH | methanol |
| min | minute(s) |
| MS | mass spectrometry |
| NaOMe | sodium methoxide |
| NMP | N-methylpyrrolidone |
| NP | normal phase |
| OTf | triflate |
| PEG | polyethylene glycol |
| Ph | phenyl |
| p.o. | per os (Latin)/by mouth |
| prep | preparative |
| PyBop | (benzotriazol-1-yl-oxytripyrrolidinophosphonium |
| | hexafluorophosphate) |
| Ref. | reference |
| RP | reverse phase |
| rpm | revolutions per minute |
| Rt | retention time |
| RM | reaction mixture |
| RT | room temperature |
| sat | saturated |
| TBAI | tetrabutylammonium iodide |
| TBME | tertiary-butyl methyl ether |
| tert | tertiary |
| TFA | trifluoroacetic acid |
| THF | tetrahydrofuran |
| TLC | thin layer chromatography |
| UPLC | ultra performance liquid chromatography |
| UV | ultraviolet |
| v | volume |
| XantPhos | 4,5-bis(diphenylphosphino)-9,9-dimethylxanthene |

### EXAMPLES AND INTERMEDIATE COMPOUNDS

All reagents were commercial grade and were used as received without further purification, unless otherwise specified. Reagent grade solvents were used, unless otherwise specified. The reactions facilitated by microwave heating were performed on a Biotage Initiator system using process vials fitted with aluminum caps and septa. Preparative low pressure chromatography was performed using a CombiFlash Companion or Combiflash RF systems equipped with RediSep or GraceResolv silica and C18 RP columns. Preparative RP HPLC was performed on either a Gilson system with a UV detector, a Teledyne Isco ACCQPrep HP125 system with 200-400 nm UV variable wavelength detector and a Purlon mass spectrometer, or an Agilent 1260 Infinity system equipped with DAD and mass-detectors. The RP HPLC systems were equipped with at least one of the following columns: an ACE-5AQ, 100×21.2 mm, 5 μm column; a Phenomenex Synergi Hydro-RP 80A AXIA, 100×21.2 mm, 4 μm column; an ACE SuperC18, 100×21.2 mm, 5 μm column; a RediSep C18Prep, 250 mm×50.0 mm, 5 μm column; or a Waters Sunfire C18 OBD Prep Column, 100A, 5 μm, 19 mm×100 mm with SunFire C18 Prep Guard Cartridge, 100A, 10 μm, 19 mm×10 mm. The purest fractions were collected, concentrated and dried under vacuum. Compounds were typically dried in a vacuum oven between 40° C. and 60° C. prior to purity analysis. Quoted yields include purity factoring when marked with a superscript $. Reactions were performed at RT unless otherwise stated. The compounds were automatically named using OpenEye rules.

Compound analysis was performed by LCMS, HPLC and UPLC. LCMS data was collected using an Agilent 1100 HPLC system with a Waters ZQ mass spectrometer connected, a Waters ACQUITY H-class UPLC with ACQUITY QDa mass detector connected, an Agilent 1100 Series LC/MSD system with DAD\ELSD and Agilent LC\MSD VL (G1956A), SL (G1956B) mass-spectrometer; or an Agilent 1200 Series LC/MSD system with DAD\ELSD and Agilent LC\MSD SL (G6130A), SL (G6140A) mass-spectrometer. The mass values reported correspond to the parent molecule with a hydrogen added [MH]⁺ or a sodium added [MNa]⁺. The HPLC and UPLC data was collected on either an Agilent 1100 system with DAD, an Agilent 1200 system with DAD, or an Agilent 1290 Infinity system with DAD. The analytical HPLC or UPLC systems utilised the following columns and methods: a Phenomenex Kinetex XB-C18 column (1.7 μm, 2.1×100 mm) at 40° C. and 0.5 mL/min with a gradient of 5% MeCN (⁺0.085% TFA) in water (⁺0.1% TFA) for 1.0 min, 5-100% over 8.0 min, holding for 0.2 min, then reequilibration for 0.8 min with a collection wavelength of 200-300 nm; a Phenomenex Kinetex XB-C18 column (1.7 μm, 2.1×50 mm) at 40° C. and 0.8 mL/min with a gradient of 5% MeCN (⁺0.085% TFA) in water (⁺0.1% TFA) for 1.0 min, 5-100% over 3.0 min, holding for 0.2 min, then reequilibration for 0.8 min with a collection wavelength of 200-300 nm; a Zorbax SB-C18 column (1.8 μm, 4.6×15 mm Rapid Resolution cartridge) at 3.0 mL/min with a gradient of 0% MeCN (0.1% formic acid) in water (0.1% formic acid) for 0.01 min, 0-100% over 1.5 min, holding for 0.3 min then reequilibration for 0.2 min, or a Phenomenex Kinetex XB-C18 column (1.7 μm, 2.1×50 mm) at 40° C. and 0.8 mL/min with a gradient of 0-20% MeCN (⁺0.085% TFA) in water (⁺0.1% TFA) over 0.9 min, ramping up to 100% in 0.1 min, holding for 0.2 min, then reequilibrate for 0.8 min with a collection wavelength of 200-300 nm.

### Intermediate 1

### Methyl 1-(2-morpholinoethyl)-6-oxo-pyridine-3-carboxylate

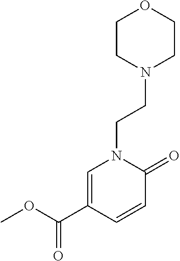 [SMILES: COC(=O)c1ccc(=O)n(CCN2CCOCC2)c1]

Methyl 6-oxo-1,6-dihydro-3-pyridinecarboxylate (123 mg, 0.80 mmol), 4-(2-chloroethyl)morpholine. HCl (223 mg, 1.20 mmol), TBAI (29.6 mg, 0.08 mmol), and K₂CO₃ (332 mg, 2.40 mmol) in MeCN (4.0 mL) were heated at 110° C. using a microwave reactor for 60 min, filtered then concentrated in vacuo. Purification by RP column chromatography gave the title compound (162 mg, 76.0%) as a colourless gum. LCMS (ES⁺): 267.1 [MH]⁺. HPLC: Rt 0.64 min, 100% purity.

### Intermediate 2

### Methyl 1-(1-methylpyrazol-3-yl)-6-oxo-pyridine-3-carboxylate

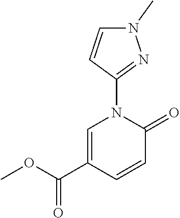 [SMILES: COC(=O)c1ccc(=O)n(-c2ccn(C)n2)c1]

1-Methyl-1H-pyrazol-3-amine (2.19 g, 22.5 mmol) and methyl coumalate (2.16 g, 20.5 mmol) in MeOH (20 mL) were stirred for 20 minutes, then the precipitate was collected by filtration and dried for 4 h under vacuum at 45° C. The crude solid was dissolved in 15% aq Na₂CO₃ (40 mL), heated to 40-45° C., stirred for 15 min, cooled to RT, and the solid material was collected by filtration then recrystallised from IPA to give the title compound (2.62 g, 57.3%) as an off-white solid. LCMS (ES⁺): 234.1 [MH]⁺.

### Intermediates 3-25

Intermediates 3-25 were prepared similarly to Intermediate 2, by ring-opening of methyl coumalate with the appropriate primary amine, followed by condensation-cyclisation; see Table 1 below.

TABLE 1
Reaction of methyl coumalate with the appropriate primary amine

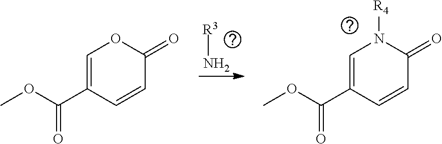

| | | | Form, Yield, |
|---|---|---|---|
| Int. | Structure | Name | LCMS, UPLC |
| 3 | 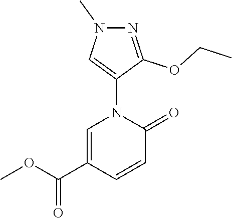 [SMILES: CCOc1nn(C)cc1-n1cc(C(=O)OC)ccc1=O] | Methyl 1-(3-ethoxy-1- methyl-pyrazol-4-yl)-6-oxo- pyridine-3-carboxylate | Light yellow solid Yield 144 mg, 53.0%^{$} LCMS (ES⁺): 278.1 [MH]⁺ UPLC: Rt 2.11 min, 99.4% purity |
| 4 | 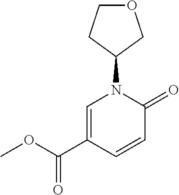 [SMILES: COC(=O)c1ccc(=O)n([C@H]2CCOC2)c1] | Methyl 6-oxo-1-[(3S)- tetrahydrofuran-3- yl]pyridine-3-carboxylate | Off-white solid Yield 80.0 mg, 18.2%^{$} LCMS (ES⁺): N/R UPLC: Rt 1.73 min, 99.0% purity |
| 5 | 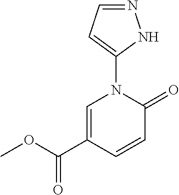 [SMILES: COC(=O)c1ccc(=O)n(-c2ccn[nH]2)c1] | Methyl 6-oxo-1-(1H- pyrazol-5-yl)pyridine-3- carboxylate | White solid Yield 857 mg, 80.3%^{$} LCMS (ES⁺): 220.1 [MH]⁺ UPLC: Rt 1.69 min, 100% purity |
| 6 | 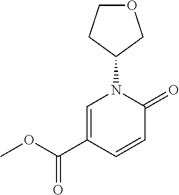 [SMILES: COC(=O)c1ccc(=O)n([C@@H]2CCOC2)c1] | Methyl 6-oxo-1-[(3R)- tetrahydrofuran-3- yl]pyridine-3-carboxylate | Off-white solid Yield 199 mg, 38.2%^{$} LCMS (ES⁺): 224.1 [MH]⁺ UPLC: Rt 1.72 min, 100% purity |
| 7 | 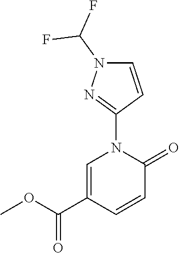 [SMILES: COC(=O)c1ccc(=O)n(-c2ccn(C(F)F)n2)c1] | Methyl 1-[1- (difluoromethyl)pyrazol-3- yl]-6-oxo-pyridine-3- carboxylate | White solid Yield: 132 mg, 5.1% LCMS (ES⁺): 270.0 [MH]⁺ |
| 8 | 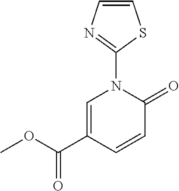 [SMILES: COC(=O)c1ccc(=O)n(-c2nccs2)c1] | Methyl 6-oxo-1-thiazol-2-yl- pyridine-3-carboxylate | Off-white solid Yield 147 mg, 31.5%^{$} LCMS (ES⁺): 236.9 [MH]⁺ UPLC: Rt 2.56 min, 98.6% purity |
| 9 | 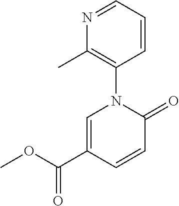 [SMILES: COC(=O)c1ccc(=O)n(-c2cccnc2C)c1] | Methyl 1-(2-methyl-3- pyridyl)-6-oxo-pyridine-3- carboxylate | Yellow gum Yield 69.0 mg, 13.6%^{$} LCMS (ES⁺): 245.1 [MH]⁺ UPLC: Rt 0.97 min, 93.4% purity |
| 10 | 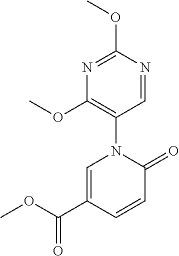 [SMILES: COC(=O)c1ccc(=O)n(-c2cnc(OC)nc2OC)c1] | Methyl 1-(2,4- dimethoxypyrimidin-5-yl)-6- oxo-pyridine-3-carboxylate | Off-white solid Yield 178 mg, 31.1% LCMS (ES⁺): 292.0 [MH]⁺ UPLC: Rt 2.01 min, 99.0% purity |
| 11 | 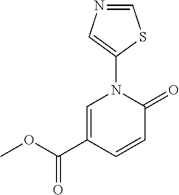 [SMILES: COC(=O)c1ccc(=O)n(-c2cncs2)c1] | Methyl 6-oxo-1-thiazol-5-yl- pyridine-3-carboxylate | Tan solid Yield 246 mg, 52.1%^{$} LCMS (ES⁺): 237.0 [MH]⁺ UPLC: Rt 1.79 min, 97.4% purity |
| 12 | 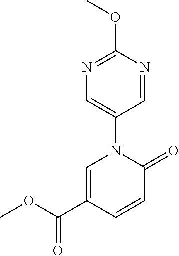 [SMILES: COC(=O)c1ccc(=O)n(-c2cnc(OC)nc2)c1] | Methyl 1-(2- methoxypyrimidin-5-yl)-6- oxo-pyridine-3-carboxylate | Off-white solid Yield 353 mg, 69.5%^{$} LCMS (ES⁺): 262.0 [MH]⁺ UPLC: Rt 1.78 min, 100% purity |
| 13 | 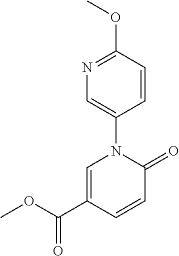 [SMILES: COC(=O)c1ccc(=O)n(-c2ccc(OC)nc2)c1] | Methyl 1-(6-methoxy-3- pyridyl)-6-oxo-pyridine-3- carboxylate | Light purple solid Yield 307 mg, 60.5%^{$} LCMS (ES⁺): 261.0 [MH]⁺ UPLC: Rt 2.01 min, 100% purity |
| 14 | 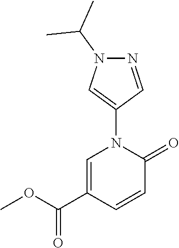 [SMILES: COC(=O)c1ccc(=O)n(-c2cnn(C(C)C)c2)c1] | Methyl 1-(1- isopropylpyrazol-4-yl)-6- oxo-pyridine-3-carboxylate | Light brown gum Yield 414 mg, 78.6%^{$} LCMS (ES⁺): 262.2 [MH]⁺ UPLC: Rt 2.17 min, 96.6% purity |
| 15 | 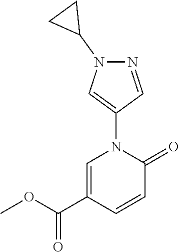 [SMILES: COC(=O)c1ccc(=O)n(-c2cnn(C3CC3)c2)c1] | Methyl 1-(1- cyclopropylpyrazol-4-yl)-6- oxo-pyridine-3-carboxylate | Light brown gum Yield 304 mg, 59.4%^{$} LCMS (ES⁺): 260.1 [MH]⁺ HPLC: Rt 2.07 min, 98.6% purity |
| 16 | 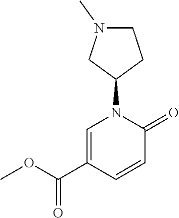 [SMILES: COC(=O)c1ccc(=O)n([C@@H]2CCN(C)C2)c1] | Methyl 1-[(3R)-1- methylpyrrolidin-3-yl]-6- oxo-pyridine-3-carboxylate | Off-white solid Yield 247 mg, 52.1%^{$} LCMS (ES⁺): 237.1 [MH]⁺ UPLC: Rt 0.64 min, 99.4% purity |
| 17 | 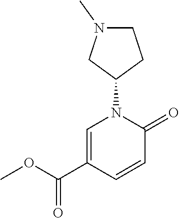 [SMILES: COC(=O)c1ccc(=O)n([C@H]2CCN(C)C2)c1] | Methyl 1-[(3S)-1- methylpyrrolidin-3-yl]-6- oxo-pyridine-3-carboxylate | Light brown solid Yield 279 mg, 58.9%^{$} LCMS (ES⁺): 237.1 [MH]⁺ UPLC: Rt 0.57 min, 99.7% purity |
| 18 | 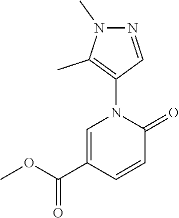 [SMILES: COC(=O)c1ccc(=O)n(-c2cnn(C)c2C)c1] | Methyl 1-(1,5- dimethylpyrazol-4-yl)-6- oxo-pyridine-3-carboxylate | Light yellow solid Yield 304 mg, 62.9%^{$} LCMS (ES⁺): 248.1 [MH]⁺ UPLC: Rt 1.84 min, 99.6% purity |
| 19 | 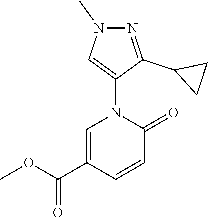 [SMILES: COC(=O)c1ccc(=O)n(-c2cn(C)nc2C2CC2)c1] | Methyl 1-(3-cyclopropyl-1- methyl-pyrazol-4-yl)-6-oxo- pyridine-3-carboxylate | Light yellow gum Yield 73.0 mg, 24.7%^{$} LCMS (ES⁺): 274.1 [MH]⁺ UPLC: Rt 2.05 min, 94.4% purity |
| 20 | 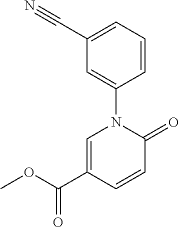 [SMILES: COC(=O)c1ccc(=O)n(-c2cccc(C#N)c2)c1] | Methyl 1-(3-cyanophenyl)- 6-oxo-pyridine-3- carboxylate | Beige solid Yield: 478 mg, 29.5% LCMS (ES⁺): 255.0 [MH]⁺ |
| 21 | 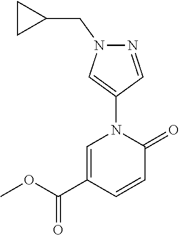 [SMILES: COC(=O)c1ccc(=O)n(-c2cnn(CC3CC3)c2)c1] | Methyl 1-[1- (cyclopropylmethyl)pyrazol- 4-yl]-6-oxo-pyridine-3- carboxylate | Orange solid Yield 224 mg, 81.5%^{$} LCMS (ES⁺): 274.1 [MH]⁺ UPLC: Rt 2.17 min, 96.8% purity |
| 22 | 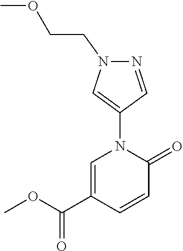 [SMILES: COCCn1cc(-n2cc(C(=O)OC)ccc2=O)cn1] | Methyl 1-[1-(2- methoxyethyl)pyrazol-4-yl]- 6-oxo-pyridine-3- carboxylate | Orange solid Yield 240 mg, 87.7%^{$} LCMS (ES⁺): 278.1 [MH]⁺ UPLC: Rt 1.89 min, 98.6% purity |
| 23 | 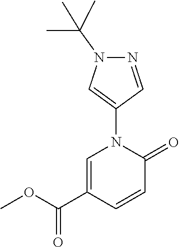 [SMILES: COC(=O)c1ccc(=O)n(-c2cnn(C(C)(C)C)c2)c1] | Methyl 1-(1-tert- butylpyrazol-4-yl)-6-oxo- pyridine-3-carboxylate | Orange gum Yield 171 mg, 63.0%^{$} LCMS (ES⁺): 276.1 [MH]⁺ UPLC: Rt 2.25 min, 98.7% purity |
| 24 | 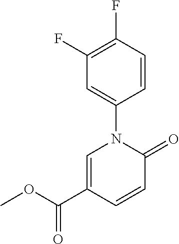 [SMILES: COC(=O)c1ccc(=O)n(-c2ccc(F)c(F)c2)c1] | Methyl 1-(3,4- difluorophenyl)-6-oxo-1,6- dihydropyridine-3- carboxylate | Off-white solid Yield: 1.08 g, 31.4% LCMS (ES⁺): 266.0 [MH]⁺ |
| 25 | 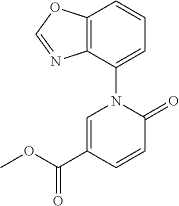 [SMILES: COC(=O)c1ccc(=O)n(-c2cccc3ocnc23)c1] | Methyl 1-(1,3-benzoxazol- 4-yl)-6-oxo-pyridine-3- carboxylate | White solid Yield 621 mg, 31.4% LCMS (ES⁺): 271.0 [MH]⁺ |

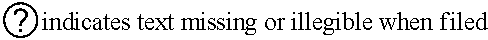

### Intermediate 26

### 1-(3,4-Dimethoxyphenyl)-6-oxo-pyridine-3-carboxylic acid

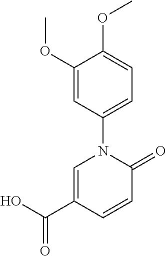 [SMILES: COc1ccc(-n2cc(C(=O)O)ccc2=O)cc1OC]

Methyl coumalate (503 mg, 3.25 mmol) and 3,4-dimethoxyaniline (510 mg, 3.24 mmol) in dry MeOH (10 mL) was refluxed for 2 h, cooled to RT then NaOH (264 mg, 6.50 mmol) was added and the RM stirred for an additional 12 h. The RM was treated with water (40 mL), washed with EtOAc (20 mL) and CHCl₃ (20 mL). The aqueous layer was acidified to pH 2 using 10% HCl and extracted by EtOAc (2×20 mL). The combined organic layers were dried over Na₂SO₄ and the solvent was evaporated. The crude product was purified by RP HPLC to give the title compound (54.0 mg, 6.2%) as a white solid. LCMS (ES⁺): 276.0 [MH]⁺.

### Intermediates 27-47

Intermediates 27-47 were prepared similarly to Intermediate 26, by ring-opening of methyl coumalate with the appropriate primary amine, followed by condensation-cyclisation and subsequent ester hydrolysis; see Table 2 below.

TABLE 2
Reaction of methyl coumalate with the appropriate primary amine,
then ester hydrolysis

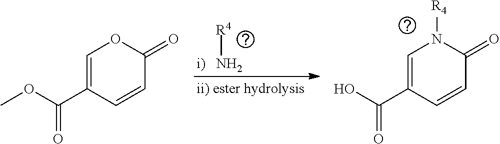

| | | | Form, Yield, |
|---|---|---|---|
| Int. | Structure | Name | LCMS, UPLC |
| 27 | 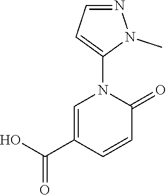 [SMILES: Cn1nccc1-n1cc(C(=O)O)ccc1=O] | 1-(2-Methylpyrazol-3- yl)-6-oxo-pyridine-3- carboxylic acid | White solid Yield: 254 mg, 12.3% LCMS (ES⁺): 220.0 [MH]⁺ |
| 28 | 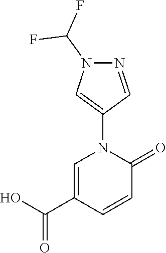 [SMILES: O=C(O)c1ccc(=O)n(-c2cnn(C(F)F)c2)c1] | 1-[1- (Difluoromethyl)pyrazol- 4-yl]-6-oxo-pyridine-3- carboxylic acid | Brown solid Yield: 516 mg, 60.1% LCMS (ES⁺): 256.0 [MH]⁺ |
| 29 | 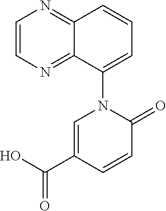 [SMILES: O=C(O)c1ccc(=O)n(-c2cccc3nccnc23)c1] | 6-Oxo-1-quinoxalin-5-yl- pyridine-3-carboxylic acid | Yellow solid Yield: 1.34 g, 42.4% LCMS (ES⁺): 268.0 [MH]⁺ |
| 30 | 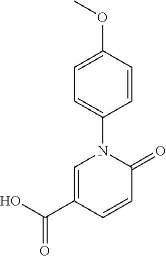 [SMILES: COc1ccc(-n2cc(C(=O)O)ccc2=O)cc1] | 1-(4-Methoxyphenyl)-6- oxo-pyridine-3- carboxylic acid | Off-white solid Yield: 2.13 g, 96.4% LCMS (ES⁺): 246.0 [MH]⁺ |
| 31 | 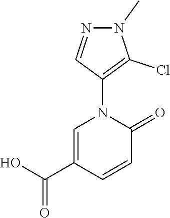 [SMILES: Cn1ncc(-n2cc(C(=O)O)ccc2=O)c1Cl] | 1-(5-Chloro-1-methyl- pyrazol-4-yl)-6-oxo- pyridine-3-carboxylic acid | Yellow solid Yield: 463 mg, 56.2% LCMS (ES⁺): 254.0 [MH]⁺ |
| 32 | 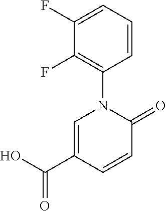 [SMILES: O=C(O)c1ccc(=O)n(-c2cccc(F)c2F)c1] | 1-(2,3-Difluorophenyl)- 6-oxo-pyridine-3- carboxylic acid | White solid Yield: 353 mg, 15.5% LCMS (ES⁺): 252.0 [MH]⁺ |
| 33 | 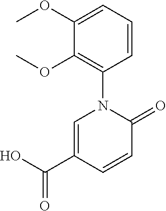 [SMILES: COc1cccc(-n2cc(C(=O)O)ccc2=O)c1OC] | 1-(2,3- Dimethoxyphenyl)-6- oxo-pyridine-3- carboxylic acid | Off-white solid Yield: 814 mg, 16.5% LCMS (ES⁺): 276.0 [MH]⁺ |
| 34 | 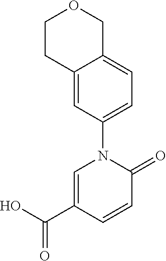 [SMILES: O=C(O)c1ccc(=O)n(-c2ccc3c(c2)CCOC3)c1] | 1-Isochroman-6-yl-6- oxo-pyridine-3- carboxylic acid | Colourless oil Yield NR LCMS (ES⁺): 272.0 [MH]⁺ |
| 35 | 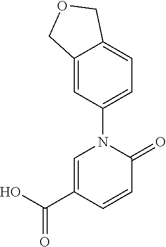 [SMILES: O=C(O)c1ccc(=O)n(-c2ccc3c(c2)COC3)c1] | 1-(1,3- Dihydroisobenzofuran- 5-yl)-6-oxo-pyridine-3- carboxylic acid | White solid Yield: 2.24 g, 66.3% LCMS (ES⁺): 258.0 [MH]⁺ |
| 36 | 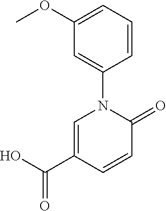 [SMILES: COc1cccc(-n2cc(C(=O)O)ccc2=O)c1] | 1-(3-Methoxyphenyl)-6- oxo-pyridine-3- carboxylic acid | Off-white solid Yield: 1.43 g, 46.4% LCMS (ES⁺): 246.0 [MH]⁺ |
| 37 | 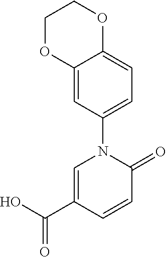 [SMILES: O=C(O)c1ccc(=O)n(-c2ccc3c(c2)OCCO3)c1] | 1-(2,3-Dihydro-1,4- benzodioxin-6-yl)-6- oxo-pyridine-3- carboxylic acid | White solid Yield: 62.0 mg, 7.1% LCMS (ES⁺): 274.0 [MH]⁺ |
| 38 | 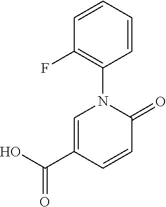 [SMILES: O=C(O)c1ccc(=O)n(-c2ccccc2F)c1] | 1-(2-Fluorophenyl)-6- oxo-pyridine-3- carboxylic acid | White solid Yield: 26.0 mg, 3.2% LCMS (ES⁺): 234.0 [MH]⁺ |
| 39 | 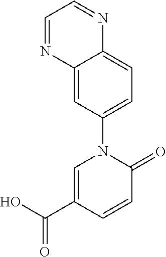 [SMILES: O=C(O)c1ccc(=O)n(-c2ccc3nccnc3c2)c1] | 6-Oxo-1-quinoxalin-6-yl- pyridine-3-carboxylic acid | Yellow solid Yield: 1.34 g, 42.4% LCMS (ES⁺): 268.0 [MH]⁺ |
| 40 | 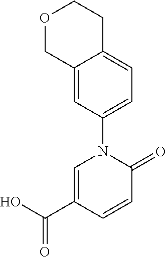 [SMILES: O=C(O)c1ccc(=O)n(-c2ccc3c(c2)COCC3)c1] | 1-Isochroman-7-yl-6- oxo-pyridine-3- carboxylic acid | White solid Yield: 1.24 g, 68.3% LCMS (ES⁺): 272.0 [MH]⁺ |
| 41 | 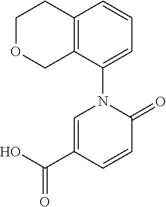 [SMILES: O=C(O)c1ccc(=O)n(-c2cccc3c2COCC3)c1] | 1-Isochroman-8-yl-6- oxo-pyridine-3- carboxylic acid | White solid Yield: 104 mg, 12.3% LCMS (ES⁺): 272.0 [MH]⁺ |
| 42 | 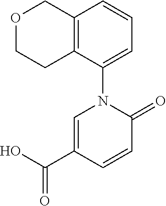 [SMILES: O=C(O)c1ccc(=O)n(-c2cccc3c2CCOC3)c1] | 1-Isochroman-5-yl-6- oxo-pyridine-3- carboxylic acid | White solid Yield: 54.0 mg, 4.1% LCMS (ES⁺): 272.0 [MH]⁺ |
| 43 | 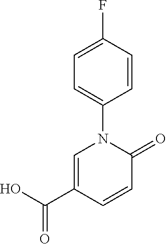 [SMILES: O=C(O)c1ccc(=O)n(-c2ccc(F)cc2)c1] | 1-(4-Fluorophenyl)-6- oxo-pyridine-3- carboxylic acid | White solid Yield: 52.0 mg, 7.4% LCMS (ES⁺): 234.0 [MH]⁺ |
| 44 | 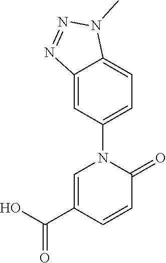 [SMILES: Cn1nnc2cc(-n3cc(C(=O)O)ccc3=O)ccc21] | 1-(1- Methylbenzotriazol-5- yl)-6-oxo-pyridine-3- carboxylic acid | Yellow solid Yield: 18.0 mg, 2.3% LCMS (ES⁺): 271.0 [MH]⁺ |
| 45 | 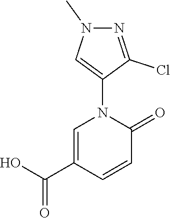 [SMILES: Cn1cc(-n2cc(C(=O)O)ccc2=O)c(Cl)n1] | 1-(3-Chloro-1-methyl- pyrazol-4-yl)-6-oxo- pyridine-3-carboxylic acid | Yellow solid Yield: 73.0 mg, 9.5% LCMS (ES⁺): 254.0 [MH]⁺ |
| 46 | 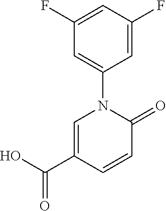 [SMILES: O=C(O)c1ccc(=O)n(-c2cc(F)cc(F)c2)c1] | 1-(3,5-Difluorophenyl)- 6-oxo-pyridine-3- carboxylic acid | White solid Yield: 63.0 mg, 4.5% LCMS (ES⁺): 252.0 [MH]⁺ |
| 47 | 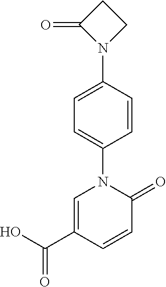 [SMILES: O=C(O)c1ccc(=O)n(-c2ccc(N3CCC3=O)cc2)c1] | 6-Oxo-1-[4-(2- oxoazetidin-1- yl)phenyl]pyridine-3- carboxylic acid | Off-white solid Yield: 587 mg, 25.8% LCMS (ES⁺): 285.0 [MH]⁺ |

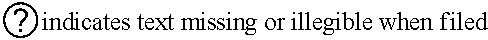

### Intermediate 48

### 5-Bromo-1-(1-methylpyrazol-4-yl)-6-oxo-pyridine-3-carboxylic acid

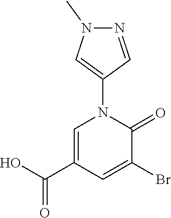 [SMILES: Cn1cc(-n2cc(C(=O)O)cc(Br)c2=O)cn1]

Intermediate 48 was prepared similarly to Intermediate 26, using methyl 3-bromo-2-oxo-2H-pyran-5-carboxylate instead of methyl coumalate and 1-methyl-1H-pyrazol-4-amine instead of 3,4-dimethoxyaniline, to give the title compound (9.54 g, 78.3%) as a yellow solid. LCMS (ES⁺): 298.0 [MH]⁺.

### Intermediate 49

### Lithium 1-(1,3-benzoxazol-4-yl)-6-oxo-pyridine-3-carboxylate

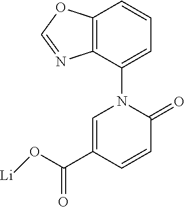

Intermediate 25 (312 mg, 1.12 mmol) and LiOH (442 mg, 1.12 mmol) were stirred in 1:1 EtOH:water (10 mL) at RT until TLC analysis (EtOAc/10% MeOH) showed full consumption of the starting material. Solvent was removed under reduced pressure and the residue was dried under vacuum giving the title compound (244 mg, 78.4%) as a yellow solid, which was used in the next step without purification or characterisation.

### Intermediate 50

### Methyl 6-methoxy-5-morpholino-pyridine-3-carboxylate

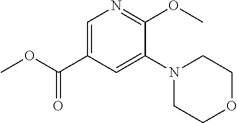 [SMILES: COC(=O)c1cnc(OC)c(N2CCOCC2)c1]

Methyl 5-bromo-6-methoxynicotinate (3.04 g, 12.2 mmol) and morpholine (2.13 g, 24.4 mmol) in dry dioxane (30 mL) were treated with Cs₂CO₃ (11.9 g, 36.6 mmol), Pd₂(dba)₃ (331 mg, 366 μmol) and XantPhos (421 mg, 732 μmol) then heated to 100° C. overnight. The RM was mixed with water (50 mL), extracted with EtOAc (3×50 mL), the combined organic extracts concentrated under vacuum to give the title compound which was used in the next step without further purification or characterisation.

### Intermediate 51

### 5-Morpholino-6-oxo-1H-pyridine-3-carboxylic acid

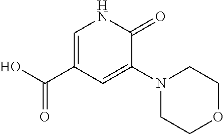 [SMILES: O=C(O)c1c[nH]c(=O)c(N2CCOCC2)c1]

Intermediate 50 (1.03 g, 3.97 mmol) and conc aq HCl (5.0 mL) was heated under reflux for 12 h, then concentrated under vacuum to give the title compound which was used in the next step without further purification or characterisation.

### Intermediate 52

### 6-Oxo-5-(1H-pyrazol-5-yl)-1H-pyridine-3-carboxylic acid

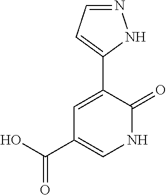 [SMILES: O=C(O)c1c[nH]c(=O)c(-c2ccn[nH]2)c1]

Methyl 5-bromo-6-methoxynicotinate (3.04 g, 12.2 mmol), 3-(tetramethyl-1,3,2-dioxaborolan-2-yl)-1H-pyrazole (3.08 g, 15.9 mmol), K₂CO₃ (6.73 g, 48.8 mmol) and Pd(dppf)Cl₂.DCM (500 mg, 0.61 mmol) in dioxane-water (100 mL, 1:1, v/v) was heated to 90° C. for 12 h. The RM was diluted with water (50 mL), extracted with EtOAc (2×30 mL) and the combined organic layers concentrated in vacuo.

The residue was treated with conc HCl solution (10 mL) and THF (10 mL), stirred at 35-40° C. for 1 h then the precipitated solid was filtered and dried to give the title product (922 mg, 36.7%) as a grey powder. LCMS (ES⁺): 206.0 [MH]⁺.

### General Amidation Procedures A, B, C, D and E

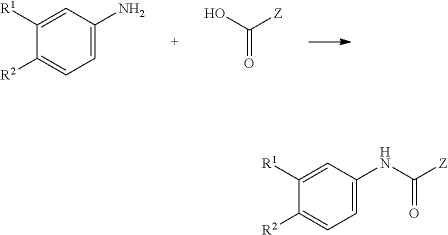

Several amidation procedures were used throughout this invention to prepare various intermediates and exemplified compounds, through reaction of the appropriate aniline with the appropriate carboxylic acid. R¹ and R² are as defined in formula (I) and Z is either A as defined in formula (I), or Z is an intermediate used in the preparation of A. General, representative amidation protocols A-E are outlined below.

### General Amidation Procedure A:

The appropriate carboxylic acid (25.8 mmol) was suspended in dry DCM (100 mL) and dry DMF (200 μL) at 0° C. then COCl₂ (2.22 mL, 25.8 mmol) was added dropwise and the mixture stirred at RT for 2 h. DIPEA (9.00 mL, 51.7 mmol) and the appropriate aniline (25.8 mmol) were added and the reaction stirred at RT for 18 h. Sat aq NaHCO₃ (50 mL) was carefully added and the mixture extracted with DCM or EtOAc (3×50 mL). The combined organic extracts were concentrated in vacuo and typically purified by column chromatography, RP HPLC, trituration or crystallisation.

### General Amidation Procedure B:

The appropriate carboxylic acid (5.91 mmol), the appropriate aniline (8.87 mmol), EDCI.HCl (2.27 g, 11.8 mmol), DMAP (72.2 mg, 591 μmol) and DIPEA (3.09 mL, 17.7 mmol) in dioxane (20 mL) was stirred under reflux until reaction completion by LCMS then concentrated in vacuo and typically purified by column chromatography, RP HPLC, trituration or crystallisation.

### General Amidation Procedure C:

The appropriate carboxylic acid was suspended in dioxane (12 mL) then HATU (466 mg, 1.22 mmol) and DIPEA (213 μL, 1.22 mmol) were added and the RM stirred at 80° C. for 15 min. The appropriate aniline (284 mg, 1.47 mmol) in dioxane (1.0 mL) was added and the RM stirred at 80° C. overnight, then concentrated in vacuo and typically purified by column chromatography, RP HPLC, trituration or crystallisation.

### General Amidation Procedure D:

The appropriate carboxylic acid (14.3 mmol, 1 equiv.) and CDI (17.2 mmol, 1.2 equiv.) in DMF (10 mL) was stirred at 80° C. for 2 h, then the required aniline (15.7 mmol, 1.1 equiv.) was added and the RM was stirred at 80° C. overnight. The RM was cooled to RT, DMF was removed under vacuum, the residue mixed with water and extracted with EtOAc (3×20 mL). The combined organic extracts were dried over Na₂SO₄, filtered and concentrated under vacuum then typically purified by column chromatography, RP HPLC, trituration or crystallisation.

### General Amidation Procedure E:

PyBop (253 mg, 1.12 mmol, 1.0 equiv.) was added to a solution of the appropriate carboxylic acid (1.12 mmol, 1.0 equiv.), the appropriate aniline (1.12 mmol, 1.0 equiv.) and DIPEA (213 mg, 1.65 mmol, 1.5 equiv.) in dry DMF (10 mL). The RM was stirred at RT overnight, then treated with water (10 mL) and extracted with EtOAc (2×10 mL). The combined organic layers were dried under Na₂SO₄, concentrated under vacuum and typically purified by column chromatography, RP HPLC, trituration or crystallisation.

### Intermediates 53-63

Intermediates 53-63 were prepared by similar procedures to General Amidation Procedures A-E between the appropriate carboxylic acid and the appropriate aniline; see Table 3 below. If no intermediates are specified, then the appropriate reactants were sourced commercially.

TABLE 3
Amidation reactions

| | | | Intermediate(s) used, |
|---|---|---|---|
| | | | Amidation procedure, |
| | | | Form, Yield, LCMS, |
| Int. | Structure | Name | UPLC |
| 53 | 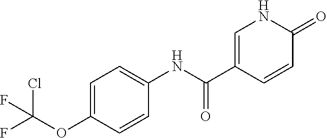 [SMILES: O=C(Nc1ccc(OC(F)(F)Cl)cc1)c1ccc(=O)[nH]c1] | N-[4- [Chloro(difluoro) methoxy]phenyl]- 6-oxo-1H- pyridine-3- carboxamide | Amidation Procedure A Off-white solid Yield 3.04 g, 37.3%^{$} LCMS (ES⁺): 315.1 [MH]⁺ UPLC: Rt 4.86 min, 99.6% purity |
| 54 | 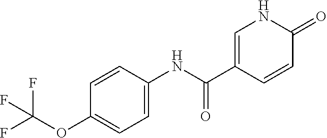 [SMILES: O=C(Nc1ccc(OC(F)(F)F)cc1)c1ccc(=O)[nH]c1] | 6-Oxo-N-[4- (trifluoromethoxy) phenyl]-1H- pyridine-3- carboxamide | Amidation Procedure D Yellow solid Yield 3.51 g, 82.0% LCMS (ES⁺): 299.0 [MH]⁺ |
| 55 | 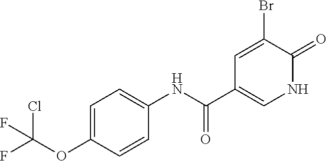 [SMILES: O=C(Nc1ccc(OC(F)(F)Cl)cc1)c1c[nH]c(=O)c(Br)c1] | 5-Bromo-N-[4- [chloro(difluoro) methoxy]phenyl]- 6-oxo-1H- pyridine-3- carboxamide | Amidation Procedure A White solid Yield 3.60 g, 63.5% LCMS (ES⁺): 393.0 [MH]⁺ UPLC: Rt 2.65 min, 89.6% purity |
| 56 | 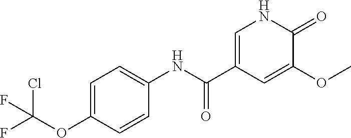 [SMILES: COc1cc(C(=O)Nc2ccc(OC(F)(F)Cl)cc2)c[nH]c1=O] | N-[4- [Chloro(difluoro) methoxy]phenyl]- 5-methoxy-6- oxo-1H-pyridine- 3-carboxamide | Amidation Procedure B White solid Yield 711 mg, 34.8%^{$} LCMS (ES⁺): 345.0 [MH]⁺ UPLC: Rt 2.47 min, 99.8% purity |
| 57 | 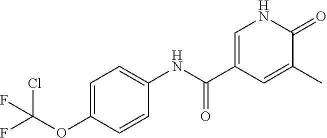 [SMILES: Cc1cc(C(=O)Nc2ccc(OC(F)(F)Cl)cc2)c[nH]c1=O] | N-[4- [Chloro(difluoro) methoxy]phenyl]- 5-methyl-6-oxo- 1H-pyridine-3- carboxamide | Amidation Procedure B Off-white solid Yield 559 mg, 27.9%^{$} LCMS (ES⁺): 329.1 [MH]⁺ UPLC: Rt 2.54 min, 98.3% purity |
| 58 | 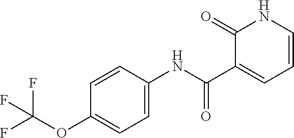 [SMILES: O=C(Nc1ccc(OC(F)(F)F)cc1)c1ccc[nH]c1=O] | 2-Oxo-N-[4- (trifluoromethoxy) phenyl]-1H- pyridine-3- carboxamide | Amidation Procedure D Light yellow solid Yield 1.76 g, 88.3% LCMS (ES⁺): 299.0 [MH]⁺ |
| 59 | 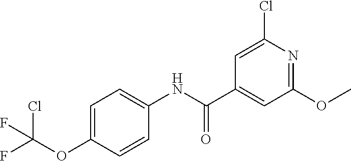 [SMILES: COc1cc(C(=O)Nc2ccc(OC(F)(F)Cl)cc2)cc(Cl)n1] | 2-Chloro-N-[4- [chloro(difluoro) methoxy]phenyl]- 6-methoxy- pyridine-4- carboxamide | Amidation Procedure D White solid Yield 1.49 g, 66.3% LCMS (ES⁺): 364.0 [MH]⁺ |
| 60 | 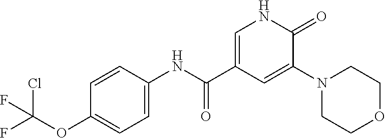 [SMILES: O=C(Nc1ccc(OC(F)(F)Cl)cc1)c1c[nH]c(=O)c(N2CCOCC2)c1] | N-[4- [Chloro(difluoro) methoxy]phenyl]- 5-morpholino-6- oxo-1H-pyridine- 3-carboxamide | Intermediate 51 Amidation Procedure D White solid Yield 253 mg, 25.1% LCMS (ES⁺): 401.0 [MH]⁺ |
| 61 | 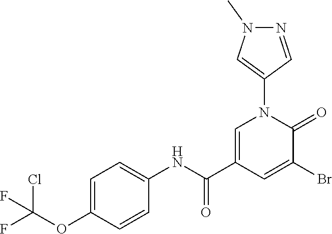 [SMILES: Cn1cc(-n2cc(C(=O)Nc3ccc(OC(F)(F)Cl)cc3)cc(Br)c2=O)cn1] | 5-Bromo-N-[4- [chloro(difluoro) methoxy]phenyl]- 1-(1- methylpyrazol-4- yl)-6-oxo- pyridine-3- carboxamide | Intermediate 48 Amidation Procedure D White solid Yield 1.22 g, 30.2% LCMS (ES⁺): 473.0 [MH]⁺ |
| 62 | 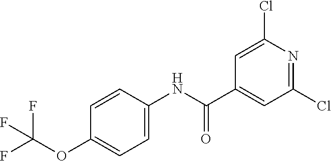 [SMILES: O=C(Nc1ccc(OC(F)(F)F)cc1)c1cc(Cl)nc(Cl)c1] | 2,6-Dichloro-N- [4- (trifluoromethoxy) phenyl]pyridine- 4-carboxamide | Amidation Procedure A Light yellow solid Yield 7.11 g, 89.3% LCMS (ES⁺): 351.0 [MH]⁺ |
| 63 | 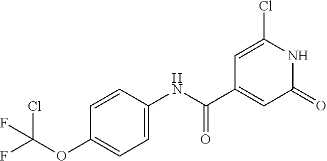 [SMILES: O=C(Nc1ccc(OC(F)(F)Cl)cc1)c1cc(Cl)[nH]c(=O)c1] | 2-Chloro-N-[4- [chloro(difluoro) methoxy]phenyl]- 6-oxo-1H- pyridine-4- carboxamide | Amidation Procedure D Yellow solid Yield 3.11 g, 75.4% LCMS (ES⁺): 350.0 [MH]⁺ |

### Intermediate 64

### 5-Bromo-N-[4-[chloro(difluoro)methoxy]phenyl]-1-methyl-6-oxo-pyridine-3-carboxamide

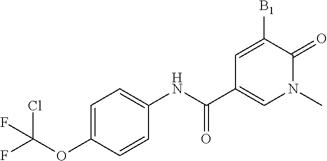

Intermediate 55 (1.70 g, 89.6% purity, 3.87 mmol), iodomethane (241 μL, 3.87 mmol) and K₂CO₃ (535 mg, 3.87 mmol) were stirred in DMSO (25 mL) for 18 h. Water (30 mL) was added to the rapidly stirring reaction mixture, which was then cooled to 0-5° C. and stirred for 10 min. The solid material was collected by filtration, washed with water (3×20 mL), then dried in the vacuum oven at 50° C. for 4 h to give the title compound (1.58 g, 94.4%^{$}) as an off-white solid. LCMS (ES⁺): 407.1 [MH]⁺. HPLC: Rt 5.71 min, 94.2% purity.

### Intermediate 65

### 2-Chloro-6-pyrimidin-5-yl-N-[4-(trifluoromethoxy)phenyl]pyridine-4-carboxamide

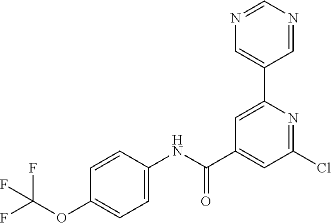 [SMILES: O=C(Nc1ccc(OC(F)(F)F)cc1)c1cc(Cl)nc(-c2cncnc2)c1]

Intermediate 62 (4.64 g, 13.0 mmol), (pyrimidin-5-yl)boronic acid (813 mg, 6.53 mmol), 25% aq K₂CO₃ (4.10 g, 29.5 mmol) and Pd(dppf)Cl₂DCM (114 mg, 0.13 mmol) in dioxane (20 mL) under argon were stirred at 90° C. for 4 h. The RM was cooled to RT, mixed with water (100 mL) and EtOAc (100 mL) and the separated organic layer was dried over Na₂SO₄ and concentrated under vacuum. Purification by column chromatography (Hex/TBME) gave the title compound (1.10 g, 35.4%) as a yellow solid. LCMS (ES⁺): 394.0 [MH]⁺.

### Intermediate 66

### 2-Methoxy-6-pyrimidin-5-yl-N-[4-(trifluoromethoxy)phenyl]pyridine-4-carboxamide

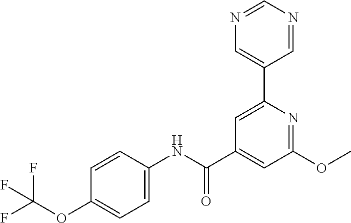 [SMILES: COc1cc(C(=O)Nc2ccc(OC(F)(F)F)cc2)cc(-c2cncnc2)n1]

MeOH (0.12 mL, 3.00 mmol) was dissolved in dry DMF (5.0 mL) followed by addition of NaH (60% in mineral oil, 61.0 mg, 1.50 mmol) then the resulting mixture stirred for 30 min at RT and 30 min at 60° C. Intermediate 65 (203 mg, 0.51 mmol) was added and the RM was left under stirring at 60° C. overnight. The RM was neutralized with few drops of AcOH, concentrated under vacuum then purified by HPLC to give the title compound (113 mg, 55.4%) as a white solid. LCMS (ES⁺): 390.0 [MH]⁺.

### Intermediate 67

### 2-Chloro-N-[4-[chloro(difluoro)methoxy]phenyl]-1-methyl-6-oxo-pyridine-4-carboxamide

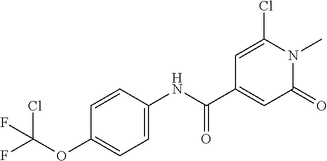 [SMILES: Cn1c(Cl)cc(C(=O)Nc2ccc(OC(F)(F)Cl)cc2)cc1=O]

Intermediate 63 (1.52 g, 4.30 mmol) was dissolved in DMF (4.0 mL) and NaH (60% in mineral oil, 213 mg, 4.70 mmol) was added. The RM was stirred for 15 min at RT then iodomethane (904 mg, 6.45 mmol) was added. The RM was heated overnight at 70° C., diluted with water (10 mL) and the precipitate was collected by filtration. Recrystallisation (1:2 EtOAc:Hex) gave the title compound (484 mg, 31.5%) as a light brown solid. LCMS (ES⁺): 364.0 [MH]⁺.

### Intermediate 68

### N-[4-[Chloro(difluoro)methoxy]phenyl]-3-iodo-benzamide

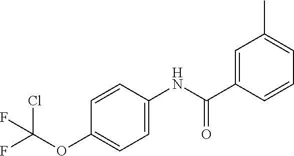 [SMILES: Cc1cccc(C(=O)Nc2ccc(OC(F)(F)Cl)cc2)c1]

Intermediate 68 was synthesised similarly to General Amidation Procedure C, using 3-iodobenzoic acid as the acid reactant, to give the title compound (3.56 g, 98.7%) as a light brown solid. LCMS (ES⁺): 423.9 [MH]⁺. UPLC: Rt 3.25 min, 98.1% purity.

### Intermediate 69

### 3-Iodo-4-methyl-N-[4-(trifluoromethoxy)phenyl]benzamide

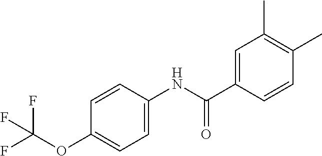 [SMILES: Cc1ccc(C(=O)Nc2ccc(OC(F)(F)F)cc2)cc1C]

Intermediate 69 was synthesised similarly to General Amidation Procedure C, using 3-iodo-4-methyl-benzoic acid as the acid reactant, to give the title compound (3.21 g, 74.7%^{$}) as a white solid. LCMS (ES⁺): 421.9 [MH]⁺. UPLC: Rt 3.29 min, 97.2% purity.

### Intermediate 70

### Methyl 5-(3-pyridyl)pyridine-3-carboxylate

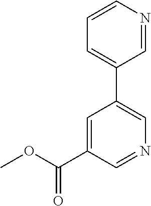 [SMILES: COC(=O)c1cncc(-c2cccnc2)c1]

Methyl 5-bromopyridine-3-carboxylate (2.08 g, 9.61 mmol) in 1,4-dioxane (20 mL) and water (1.5 mL) was degassed with N₂ for 5 min. 3-(4,4,5,5-Tetramethyl-1,3,2-dioxaborolan-2-yl)pyridine (2.07 g, 10.1 mmol), Pd(PPh3)4 (1.11 g, 0.96 mmol), and K2CO3 (1.99 g, 14.4 mmol) were added, and heated at 80° C. for 20 h. The mixture was allowed to cool to RT, filtered, and concentrated in vacuo. The crude material was treated with 2M HCl (40 mL) and EtOAc (160 mL), the aqueous layer was separated, and added to EtOAc (200 mL). The mixture was neutralised with Na2CO3 to ˜pH 7, and the phases separated. The organic phase was dried (MgSO4) and concentrated in vacuo to give the title compound (1.78 g, 86.4%^{$}) as an off-white solid. LCMS (ES⁺): 215.1 [MH]⁺. UPLC: Rt 1.99 min, 98.3% purity.

### Intermediate 71

### 5-(3-Pyridyl)pyridine-3-carboxylic acid

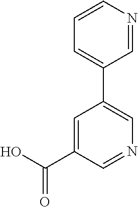 [SMILES: O=C(O)c1cncc(-c2cccnc2)c1]

Intermediate 70 (1.78 g, 8.30 mmol) was dissolved in THF (10 mL) and water (10 mL), cooled to 0° C., LiOH (522 mg, 12.5 mmol) was added, and stirred for 30 min. The organics were evaporated, the aqueous phase was diluted with water (20 mL), and washed with EtOAc (20 mL). The aqueous phase was acidified with aq KHSO4 (1M) until pH-4, the precipitate was collected by filtration, and washed

with water to give the title compound (929 mg, 55.9%^{$}) as a white solid. LCMS (ES⁺): 201.1 [MH]⁺. UPLC: Rt 1.20 min, 100% purity.

### Example 1

### N-[4-[Chloro(difluoro)methoxy]phenyl]-1-(3-ethoxy-1-methyl-pyrazol-4-yl)-6-oxo-pyridine-3-carboxamide

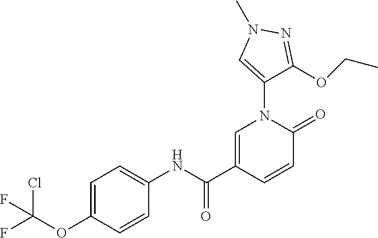 [SMILES: CCOc1nn(C)cc1-n1cc(C(=O)Nc2ccc(OC(F)(F)Cl)cc2)ccc1=O]

Intermediate 3 (144 mg, 99.4% purity, 516 μmol) and 1M aq NaOH (568 μL, 568 μmol) was stirred in THF:water (1:1, 4.0 mL) for 1 h. 1M aq HCl (568 μL, 568 μmol) was added then the mixture was concentrated in vacuo and dried in a vacuum oven at 60° C. overnight. The residue was reacted with 4-[chloro(difluoro)methoxy]aniline (120 mg, 620 μmol) similarly to General

Amidation Procedure C. Purification by RP HPLC and trituration (MeOH:water) gave the title compound (62.1 mg, 27.3%^{$}) as a white solid. LCMS (ES⁺): 439.0 [MH]⁺. UPLC: Rt 5.80 min, 99.4% purity.

### Examples 2-24

Examples 2-24 were prepared similarly to Example 1, by ester hydrolysis of Intermediates 1, 2 and 4-24 then amide coupling with the appropriate aniline similarly to General Amidation Procedures A-E; see Table 4 below.

TABLE 4
Ester hydrolysis then amide coupling reactions

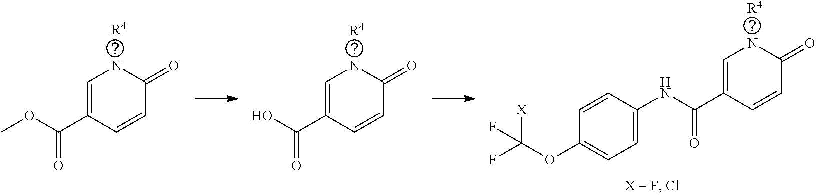

| | | | Intermediate(s) used, |
|---|---|---|---|
| | | | Amidation procedure, |
| | | | Form, Yield, LCMS, |
| Ex. | Structure | Name | UPLC |
| 2 | 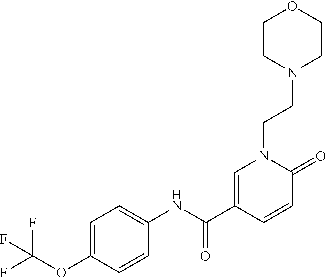 [SMILES: O=C(Nc1ccc(OC(F)(F)F)cc1)c1ccc(=O)n(CCN2CCOCC2)c1] | 1-(2- Morpholinoethyl)- 6-oxo-N-[4- (trifluoromethoxy) phenyl] pyridine-3- carboxamide | From Intermediate 1 Amidation procedure C White solid Yield 133 mg, 53.1%^{$} LCMS (ES⁺): 412.1 [MH]⁺ UPLC: Rt 4.20 min, 100% purity |
| 3 | 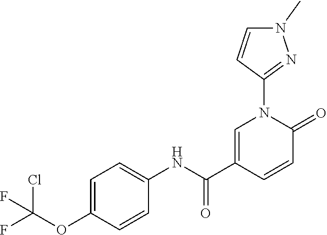 [SMILES: Cn1ccc(-n2cc(C(=O)Nc3ccc(OC(F)(F)Cl)cc3)ccc2=O)n1] | N-[4- (Chlorodifluoro- methoxy)phenyl]-1- (1-methyl-1H- pyrazol-3-yl)-6- oxo-1,6- dihydropyridine-3- carboxamide | From Intermediate 2 Amidation procedure D Off-white solid Yield: 173 mg, 18.0% LCMS (ES⁺): 395.0 [MH]⁺ UPLC: Rt 5.50 min, 99.8% purity |
| 4 | 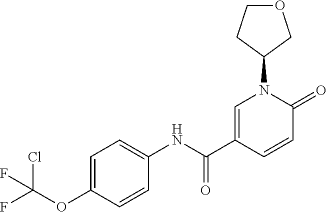 [SMILES: O=C(Nc1ccc(OC(F)(F)Cl)cc1)c1ccc(=O)n([C@H]2CCOC2)c1] | N-[4- [Chloro(difluoro) methoxy]phenyl]-6- oxo-1-[(3S)- tetrahydrofuran-3- yl]pyridine-3- carboxamide | From Intermediate 4 Amidation procedure B White solid Yield 15.0 mg, 10.9%^{$} LCMS (ES⁺): 385.0 [MH]⁺ UPLC: Rt 5.34 min, 99.5% purity |
| 5 | 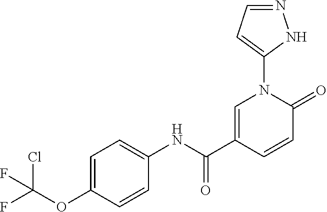 [SMILES: O=C(Nc1ccc(OC(F)(F)Cl)cc1)c1ccc(=O)n(-c2ccn[nH]2)c1] | N-[4- [Chloro(difluoro) methoxy]phenyl]-6- oxo-1-(1H- pyrazol-5- yl)pyridine-3- carboxamide | From Intermediate 5 Amidation procedure A White solid Yield 13.8 mg, 4.0%^{$} LCMS (ES⁺): 381.0 [MH]⁺ UPLC: Rt 5.19 min, 99.8% purity |
| 6 | 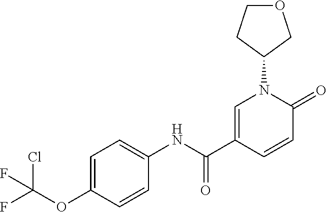 [SMILES: O=C(Nc1ccc(OC(F)(F)Cl)cc1)c1ccc(=O)n([C@@H]2CCOC2)c1] | N-[4- [Chloro(difluoro) methoxy]phenyl]-6- oxo-1-[(3R)- tetrahydrofuran-3- yl]pyridine-3- carboxamide | From Intermediate 6 Amidation procedure B Off-white solid Yield 122 mg, 35.4%^{$} LCMS (ES⁺): 385.0 [MH]⁺ UPLC: Rt 5.33 min, 100% purity |
| 7 | 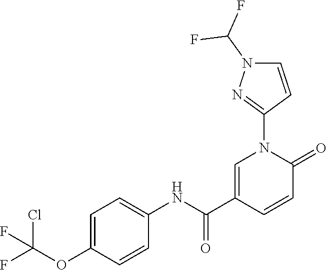 [SMILES: O=C(Nc1ccc(OC(F)(F)Cl)cc1)c1ccc(=O)n(-c2ccn(C(F)F)n2)c1] | N-[4- (Chlorodifluoro- methoxy)phenyl]-1- [1-(difluoromethyl)- 1H-pyrazol-3-yl]-6- oxo-1,6- dihydropyridine-3- carboxamide | From Intermediate 7 Amidation procedure D Off-white solid Yield: 63.0 mg, 36.4% LCMS (ES⁺): 431.0 [MH]⁺ UPLC: Rt 5.93 min, 100% purity |
| 8 | 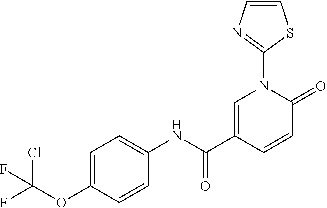 [SMILES: O=C(Nc1ccc(OC(F)(F)Cl)cc1)c1ccc(=O)n(-c2nccs2)c1] | N-[4- [Chloro(difluoro) methoxy]phenyl]-6- oxo-1-thiazol-2-yl- pyridine-3- carboxamide | From Intermediate 8 Amidation procedure B White solid Yield 16.4 mg, 6.59%^{$} LCMS (ES⁺): 397.9 [MH]⁺ UPLC: Rt 6.23 min, 98.1% purity |
| 9 | 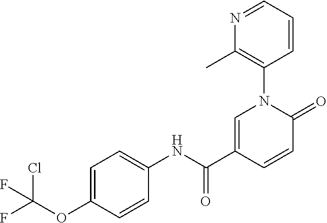 [SMILES: Cc1ncccc1-n1cc(C(=O)Nc2ccc(OC(F)(F)Cl)cc2)ccc1=O] | N-[4- [Chloro(difluoro) methoxy]phenyl]-1- (2-methyl-3- pyridyl)-6-oxo- pyridine-3- carboxamide | From Intermediate 9 Amidation procedure C Off-white solid Yield 14.8 mg, 13.7%^{$} LCMS (ES⁺): 406.0 [MH]⁺ UPLC: Rt 4.81 min, 99.1% purity |
| 10 | 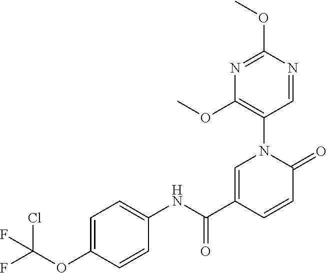 [SMILES: COc1ncc(-n2cc(C(=O)Nc3ccc(OC(F)(F)Cl)cc3)ccc2=O)c(OC)n1] | N-[4- [Chloro(difluoro) methoxy]phenyl]-1- (2,4- dimethoxypyrimidin- 5-yl)-6-oxo- pyridine-3- carboxamide | From Intermediate 10 Amidation procedure C White solid Yield 57.9 mg, 21.0% LCMS (ES⁺): 453.0 [MH]⁺ UPLC: Rt 5.82 min, 99.5% purity |
| 11 | 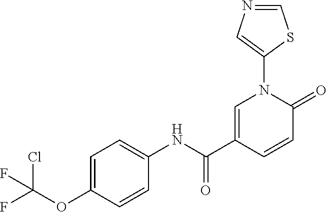 [SMILES: O=C(Nc1ccc(OC(F)(F)Cl)cc1)c1ccc(=O)n(-c2cncs2)c1] | N-[4- [Chloro(difluoro) methoxy]phenyl]-6- oxo-1-thiazol-5-yl- pyridine-3- carboxamide | From Intermediate 11 Amidation procedure C White solid Yield 102 mg, 25.0%^{$} LCMS (ES⁺): 397.9 [MH]⁺ UPLC: Rt 5.58 min, 99.5% purity |
| 12 | 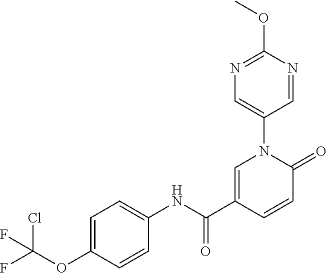 [SMILES: COc1ncc(-n2cc(C(=O)Nc3ccc(OC(F)(F)Cl)cc3)ccc2=O)cn1] | N-[4- [Chloro(difluoro) methoxy]phenyl]-1- (2- methoxypyrimidin- 5-yl)-6-oxo- pyridine-3- carboxamide | From Intermediate 12 Amidation procedure C White solid Yield 215 mg, 37.4%^{$} LCMS (ES⁺): 423.0 [MH]⁺ UPLC: Rt 5.59 min, 99.7% purity |
| 13 | 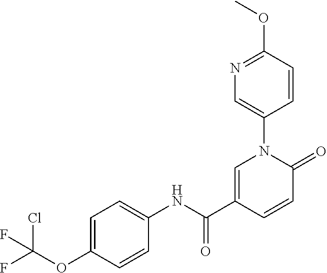 [SMILES: COc1ccc(-n2cc(C(=O)Nc3ccc(OC(F)(F)Cl)cc3)ccc2=O)cn1] | N-[4- [Chloro(difluoro) methoxy]phenyl]-1- (6-methoxy-3- pyridyl)-6-oxo- pyridine-3- carboxamide | From Intermediate 13 Amidation procedure C White solid Yield 218 mg, 43.7%^{$} LCMS (ES⁺): 422.0 [MH]⁺ UPLC: Rt 5.93 min, 99.6% purity |
| 14 | 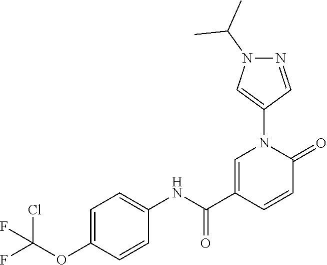 [SMILES: CC(C)n1cc(-n2cc(C(=O)Nc3ccc(OC(F)(F)Cl)cc3)ccc2=O)cn1] | N-[4- [Chloro(difluoro) methoxy]phenyl]-1- (1- isopropylpyrazol- 4-yl)-6-oxo- pyridine-3- carboxamide | From Intermediate 14 Amidation procedure C White solid Yield 251 mg, 38.6%^{$} LCMS (ES⁺): 423.0 [MH]⁺ UPLC: Rt 5.33 min, 99.6% purity |
| 15 | 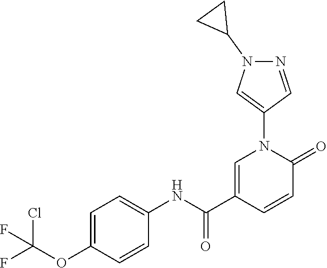 [SMILES: O=C(Nc1ccc(OC(F)(F)Cl)cc1)c1ccc(=O)n(-c2cnn(C3CC3)c2)c1] | N-[4- [Chloro(difluoro) methoxy]phenyl]-1- (1- cyclopropylpyrazol- 4-yl)-6-oxo- pyridine-3- carboxamide | From Intermediate 15 Amidation procedure C White solid Yield 259 mg, 52.2%^{$} LCMS (ES⁺): 421.0 [MH]⁺ UPLC: Rt 5.18 min, 99.4% purity |
| 16 | 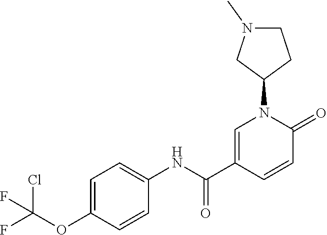 [SMILES: CN1CC[C@@H](n2cc(C(=O)Nc3ccc(OC(F)(F)Cl)cc3)ccc2=O)C1] | N-[4- [Chloro(difluoro) methoxy]phenyl]-1- [(3R)-1- methylpyrrolidin-3- yl]-6-oxo-pyridine- 3-carboxamide | From Intermediate 16 Amidation procedure C Colourless gum Yield 53.4 mg, 12.7%^{$} LCMS (ES⁺): 398.0 [MH]⁺ UPLC: Rt 4.81 min, 98.5% purity |
| 17 | 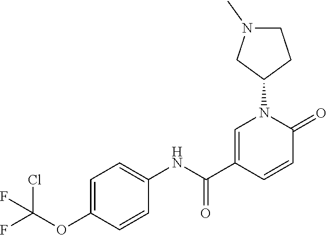 [SMILES: CN1CC[C@H](n2cc(C(=O)Nc3ccc(OC(F)(F)Cl)cc3)ccc2=O)C1] | N-[4- [Chloro(difluoro) methoxy]phenyl]-1- [(3S)-1- methylpyrrolidin-3- yl]-6-oxo-pyridine- 3-carboxamide | From Intermediate 17 Amidation procedure C Colourless gum Yield 34.0 mg, 7.1%^{$} LCMS (ES⁺): 398.1 [MH]⁺ UPLC: Rt 4.48 min, 97.4% purity |
| 18 | 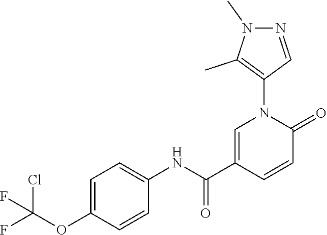 [SMILES: Cc1c(-n2cc(C(=O)Nc3ccc(OC(F)(F)Cl)cc3)ccc2=O)cnn1C] | N-[4- [Chloro(difluoro) methoxy]phenyl]-1- (1,5- dimethylpyrazol-4- yl)-6-oxo-pyridine- 3-carboxamide | From Intermediate 18 Amidation procedure C Off-white solid Yield 233 mg, 46.6%^{$} LCMS (ES⁺): 409.0 [MH]⁺ UPLC: Rt 5.38 min, 100% purity |
| 19 | 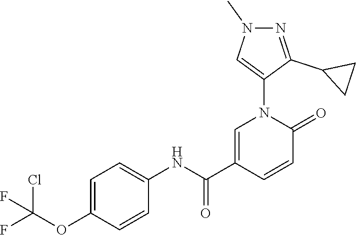 [SMILES: Cn1cc(-n2cc(C(=O)Nc3ccc(OC(F)(F)Cl)cc3)ccc2=O)c(C2CC2)n1] | N-[4- [Chloro(difluoro) methoxy]phenyl]-1- (3-cyclopropyl-1- methyl-pyrazol-4- yl)-6-oxo-pyridine- 3-carboxamide | From Intermediate 19 Amidation procedure C Off-white solid Yield 53.7 mg, 48.9%^{$} LCMS (ES⁺): 435.0 [MH]⁺ UPLC: Rt 5.73 min, 99.9% purity |
| 20 | 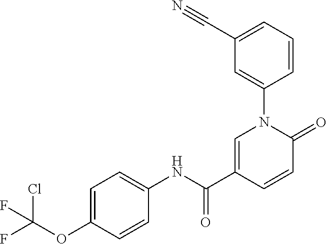 [SMILES: N#Cc1cccc(-n2cc(C(=O)Nc3ccc(OC(F)(F)Cl)cc3)ccc2=O)c1] | N-[4- (Chlorodifluoro- methoxy)phenyl]-1-(3- cyanophenyl)-6- oxo-1,6- dihydropyridine-3- carboxamide | From Intermediate 20 Amidation procedure D Brown solid Yield: 53.0 mg, 10.9% LCMS (ES⁺): 416.0 [MH]⁺ UPLC: Rt 2.84 min, 98.4% purity |
| 21 | 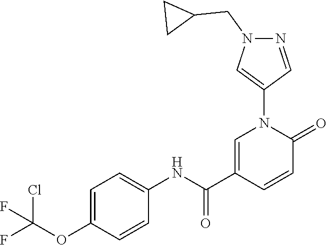 [SMILES: O=C(Nc1ccc(OC(F)(F)Cl)cc1)c1ccc(=O)n(-c2cnn(CC3CC3)c2)c1] | N-[4- [Chloro(difluoro) methoxy]phenyl]-1- [1- (cyclopropylmethyl) pyrazol-4-yl]-6- oxo-pyridine-3- carboxamide | From Intermediate 21 Amidation procedure C Off-white solid Yield 183 mg, 52.7%^{$} LCMS (ES⁺): 435.0 [MH]⁺ UPLC: Rt 5.95 min, 99.4% purity |
| 22 | 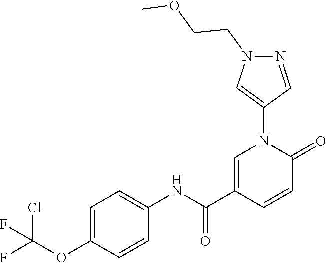 [SMILES: COCCn1cc(-n2cc(C(=O)Nc3ccc(OC(F)(F)Cl)cc3)ccc2=O)cn1] | N-[4- [Chloro(difluoro) methoxy]phenyl]-1- [1-(2- methoxyethyl)pyrazol- 4-yl]-6-oxo- pyridine-3- carboxamide | From Intermediate 22 Amidation procedure C Off-white solid Yield 156 mg, 41.5%^{$} LCMS (ES⁺): 439.0 [MH]⁺ UPLC: Rt 5.48 min, 99.7% purity |
| 23 | 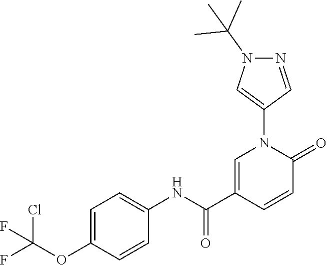 [SMILES: CC(C)(C)n1cc(-n2cc(C(=O)Nc3ccc(OC(F)(F)Cl)cc3)ccc2=O)cn1] | 1-(1-tert- Butylpyrazol-4-yl)- N-[4- [chloro(difluoro) methoxy]phenyl]-6- oxo-pyridine-3- carboxamide | From Intermediate 23 Amidation procedure C White solid Yield 112 mg, 41.9%^{$} LCMS (ES⁺): 437.1 [MH]⁺ UPLC: Rt 6.14 min, 100% purity |
| 24 | 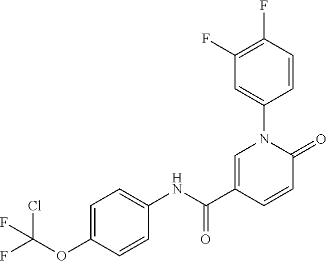 [SMILES: O=C(Nc1ccc(OC(F)(F)Cl)cc1)c1ccc(=O)n(-c2ccc(F)c(F)c2)c1] | N-[4- (Chlorodifluoro- methoxy)phenyl]-1- (3,4- difluorophenyl)-6- oxo-1,6- dihydropyridine-3- carboxamide | From Intermediate 24 Amidation procedure D Off-white solid Yield: 55.0 mg, 13.8% LCMS (ES⁺): 427.0 [MH]⁺ UPLC: Rt 2.93 min, 99.7% purity |

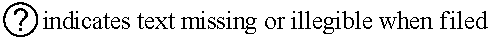

### Example 25

### N-[4-(Chlorodifluoromethoxy)phenyl]-1-(1-methyl-1H-pyrazol-5-yl)-6-oxo-1,6-dihydropyridine-3-carboxamide

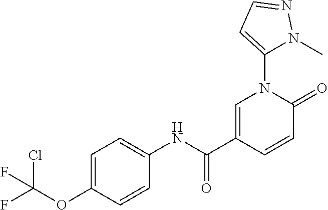 [SMILES: Cn1nccc1-n1cc(C(=O)Nc2ccc(OC(F)(F)Cl)cc2)ccc1=O]

Intermediate 27 (248 mg, 1.14 mmol) was reacted with 4-[chloro(difluoro)methoxy]aniline (263 mg, 1.14 mmol) similarly to General Amidation Procedure D. Purification by RP HPLC gave the title compound (132 mg, 29.2%) as a white solid. LCMS (ES⁺): 395.2 [MH]⁺. UPLC: Rt 5.38 min, 99.9% purity.

### Examples 26-49

Examples 26-49 were prepared similarly to Example 25, by amide coupling of the appropriate carboxylic acid with the appropriate aniline using procedures similar to General Amidation Procedure A-E, see Table 5 below. If no intermediates are specified, then commercial reactants were used.

TABLE 5
Amidation reactions

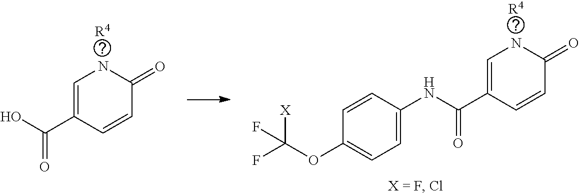

| | | | Intermediate(s) used |
|---|---|---|---|
| | | | Amidation procedure, |
| | | | Form, Yield, LCMS, |
| Ex. | Structure | Name | UPLC |
| 26 | 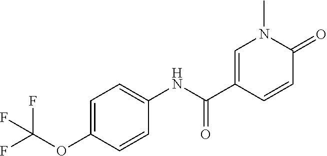 [SMILES: Cn1cc(C(=O)Nc2ccc(OC(F)(F)F)cc2)ccc1=O] | 1-Methyl-6-oxo-N-[4- (trifluoromethoxy) phenyl]pyridine-3- carboxamide | Amidation procedure C White solid Yield: 103 mg, 59.1% LCMS (ES⁺): 313.0 [MH]⁺ UPLC: Rt 4.91 min, 100% purity |
| 27 | 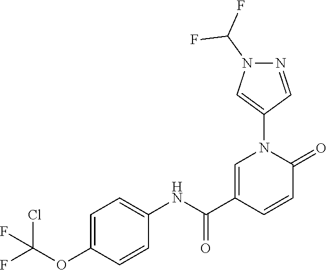 [SMILES: O=C(Nc1ccc(OC(F)(F)Cl)cc1)c1ccc(=O)n(-c2cnn(C(F)F)c2)c1] | N-[4- (Chlorodifluoro- methoxy)phenyl]-1-[1- (difluoromethyl)-1H- pyrazol-4-yl]-6-oxo- 1,6-dihydropyridine- 3-carboxamide | From Intermediate 28 Amidation procedure D Brown solid Yield: 55.0 mg, 20.4% LCMS (ES⁺): 431.0 [MH]⁺ UPLC: Rt 5.76 min, 99.2% purity |
| 28 | 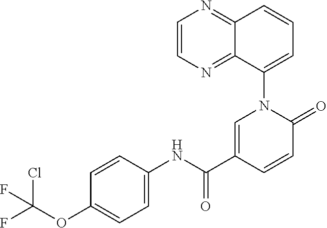 [SMILES: O=C(Nc1ccc(OC(F)(F)Cl)cc1)c1ccc(=O)n(-c2cccc3nccnc23)c1] | N-[4- (Chlorodifluoro- methoxy)phenyl]-6-oxo- 1-(quinoxalin-5-yl)-1,6- dihydropyridine-3- carboxamide | From Intermediate 29 Amidation procedure D Yellow solid Yield: 43.0 mg, 9.3% LCMS (ES⁺): 443.0 [MH]⁺ UPLC: Rt 2.70 min, 99.0% purity |
| 29 | 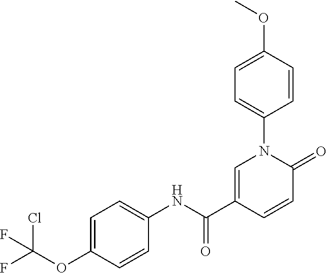 [SMILES: COc1ccc(-n2cc(C(=O)Nc3ccc(OC(F)(F)Cl)cc3)ccc2=O)cc1] | N-[4- (Chlorodifluoro- methoxy)phenyl]-1-(4- methoxyphenyl)-6- oxo-1,6- dihydropyridine-3- carboxamide | From Intermediate 30 Amidation procedure D Off-white solid Yield: 43.0 mg, 13.2% LCMS (ES⁺): 421.0 [MH]⁺ UPLC: Rt 2.90 min, 99.2% purity |
| 30 | 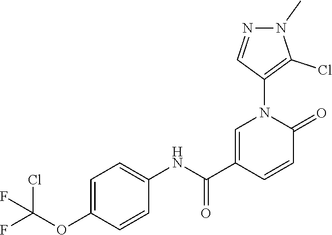 [SMILES: Cn1ncc(-n2cc(C(=O)Nc3ccc(OC(F)(F)Cl)cc3)ccc2=O)c1Cl] | 1-(5-Chloro-1- methy-1H-pyrazol- 4-yl)-N-[4- (chlorodifluoro- methoxy)phenyl]-6-oxo- 1,6-dihydropyridine- 3-carboxamide | From Intermediate 31 Amidation procedure C Brown solid Yield: 61.0 mg, 8.5% LCMS (ES⁺): 429.0 [MH]⁺ UPLC: Rt 2.74 min, 98.5% purity |
| 31 | 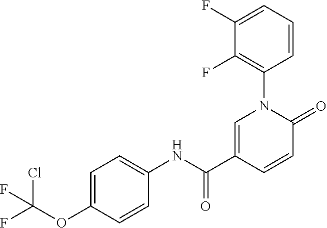 [SMILES: O=C(Nc1ccc(OC(F)(F)Cl)cc1)c1ccc(=O)n(-c2cccc(F)c2F)c1] | N-[4- (Chlorodifluoro- methoxy)phenyl]-1-(2,3- difluorophenyl)-6- oxo-1,6- dihydropyridine-3- carboxamide | From Intermediate 32 Amidation procedure D White solid Yield: 43.0 mg, 14.4% LCMS (ES⁺): 427.0 [MH]⁺ UPLC: Rt 2.93 min, 100% purity |
| 32 | 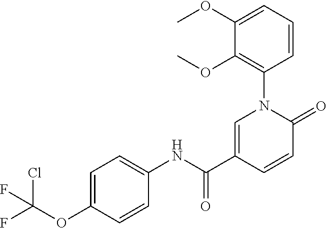 [SMILES: COc1cccc(-n2cc(C(=O)Nc3ccc(OC(F)(F)Cl)cc3)ccc2=O)c1OC] | N-[4- (Chlorodifluoro- methoxy)phenyl]-1-(2,3- dimethoxyphenyl)-6- oxo-1,6- dihydropyridine-3- carboxamide | From Intermediate 33 Amidation procedure D White solid Yield: 5.0 mg, 6.4% LCMS (ES⁺): 451.0 [MH]⁺ UPLC: Rt 2.90 min, 100% purity |
| 33 | 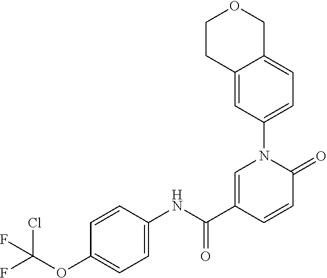 [SMILES: O=C(Nc1ccc(OC(F)(F)Cl)cc1)c1ccc(=O)n(-c2ccc3c(c2)CCOC3)c1] | N-[4- (Chlorodifluoro- methoxy)phenyl]-1-(3,4- dihydro-1H-2- benzopyran-6-yl)-6- oxo-1,6- dihydropyridine-3- carboxamide | From Intermediate 34 Amidation procedure D White solid Yield: 58.0 mg, 36.2% LCMS (ES⁺): 447.0 [MH]⁺ UPLC: Rt 2.86 min, 99.4% purity |
| 34 | 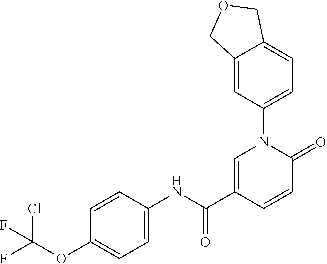 [SMILES: O=C(Nc1ccc(OC(F)(F)Cl)cc1)c1ccc(=O)n(-c2ccc3c(c2)COC3)c1] | N-[4- (Chlorodifluoro- methoxy)phenyl]-1-(1,3- dihydro-2- benzofuran-5-yl)-6- oxo-1,6- dihydropyridine-3- carboxamide | From Intermediate 35 Amidation procedure D White solid Yield: 41.0 mg, 11.4% LCMS (ES⁺): 433.0 [MH]⁺ UPLC: Rt 2.80 min, 96.9% purity |
| 35 | 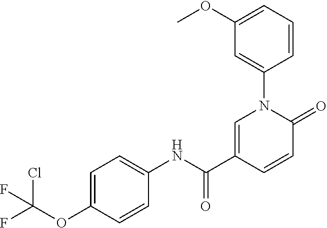 [SMILES: COc1cccc(-n2cc(C(=O)Nc3ccc(OC(F)(F)Cl)cc3)ccc2=O)c1] | N-[4- (Chlorodifluoro- methoxy)phenyl]-1-(3- methoxyphenyl)-6- oxo-1,6- dihydropyridine-3- carboxamide | From Intermediate 36 Amidation procedure D Beige solid Yield: 73.0 mg, 21.3% LCMS (ES⁺): 421.0 [MH]⁺ UPLC: Rt 2.90 min, 100% purity |
| 36 | 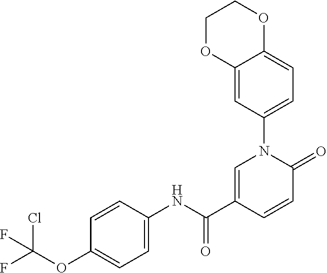 [SMILES: O=C(Nc1ccc(OC(F)(F)Cl)cc1)c1ccc(=O)n(-c2ccc3c(c2)OCCO3)c1] | N-[4- (Chlorodifluoro- methoxy)phenyl]-1-(2,3- dihydro-1,4- benzodioxin-6-yl)-6- oxo-1,6- dihydropyridine-3- carboxamide | From Intermediate 37 Amidation procedure C Yellow solid Yield: 37.0 mg, 36.3% LCMS (ES⁺): 449.0 [MH]⁺ UPLC: Rt 2.89 min, 99.2% purity |
| 37 | 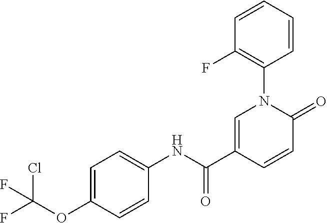 [SMILES: O=C(Nc1ccc(OC(F)(F)Cl)cc1)c1ccc(=O)n(-c2ccccc2F)c1] | N-[4- (Chlorodifluoro- methoxy)phenyl]-1-(2- fluorophenyl)-6-oxo- 1,6-dihydropyridine- 3-carboxamide | From Intermediate 38 Amidation procedure C Yellow solid Yield: 9.0 mg, 19.3% LCMS (ES⁺): 409.0 [MH]⁺ UPLC: Rt 2.88 min, 98.2% purity |
| 38 | 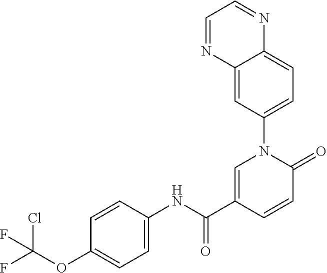 [SMILES: O=C(Nc1ccc(OC(F)(F)Cl)cc1)c1ccc(=O)n(-c2ccc3nccnc3c2)c1] | N-[4- (Chlorodifluoro- methoxy)phenyl]-6-oxo- 1-(quinoxalin-6-yl)-1,6- dihydropyridine-3- carboxamide | From Intermediate 39 Amidation procedure D Brown solid Yield: 13.0 mg, 3.4% LCMS (ES⁺): 443.2 [MH]⁺ UPLC: Rt 2.73 min, 98.8% purity |
| 39 | 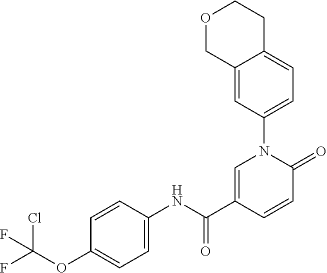 [SMILES: O=C(Nc1ccc(OC(F)(F)Cl)cc1)c1ccc(=O)n(-c2ccc3c(c2)COCC3)c1] | N-[4- (Chlorodifluoro- methoxy)phenyl]-1-(3,4- dihydro-1H-2- benzopyran-7-yl)-6- oxo-1,6- dihydropyridine-3- carboxamide | From Intermediate 40 Amidation procedure D White solid Yield: 58.0 mg, 17.1% LCMS (ES⁺): 447.0 [MH]⁺ UPLC: Rt 2.87 min, 98.4% purity |
| 40 | 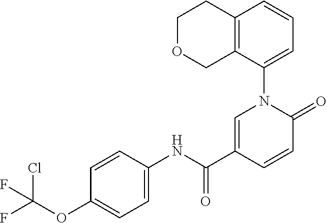 [SMILES: O=C(Nc1ccc(OC(F)(F)Cl)cc1)c1ccc(=O)n(-c2cccc3c2COCC3)c1] | N-[4- (Chlorodifluoro- methoxy)phenyl]-1-(3,4- dihydro-1H-2- benzopyran-8-yl)-6- oxo-1,6- dihydropyridine-3- carboxamide | From Intermediate 41 Amidation procedure C White solid Yield: 64.0 mg, 37.5% LCMS (ES⁺): 447.0 [MH]⁺ UPLC: Rt 2.88 min, 100% purity |
| 41 | 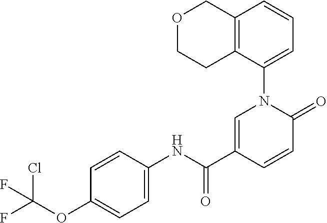 [SMILES: O=C(Nc1ccc(OC(F)(F)Cl)cc1)c1ccc(=O)n(-c2cccc3c2CCOC3)c1] | N-[4- (Chlorodifluoro- methoxy)phenyl]-1-(3,4- dihydro-1H-2- benzopyran-5-yl)-6- oxo-1,6- dihydropyridine-3- carboxamide | From Intermediate 42 Amidation procedure C White solid Yield: 29.0 mg, 33.3% LCMS (ES⁺): 447.0 [MH]⁺ UPLC: Rt 2.85 min, 99.0% purity |
| 42 | 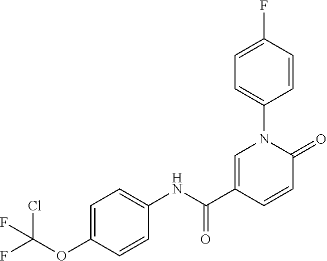 [SMILES: O=C(Nc1ccc(OC(F)(F)Cl)cc1)c1ccc(=O)n(-c2ccc(F)cc2)c1] | N-[4- (Chlorodifluoro- methoxy)phenyl]-1-(4- fluorophenyl)-6-oxo- 1,6-dihydropyridine- 3-carboxamide | From Intermediate 43 Amidation procedure C White solid Yield: 23.0 mg, 24.3% LCMS (ES⁺): 409.0 [MH]⁺ UPLC: Rt 2.89 min, 99.4% purity |
| 43 | 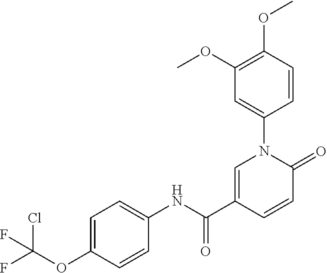 [SMILES: COc1ccc(-n2cc(C(=O)Nc3ccc(OC(F)(F)Cl)cc3)ccc2=O)cc1OC] | N-[4- (Chlorodifluoro- methoxy)phenyl]-1-(3,4- dimethoxyphenyl)-6- oxo-1,6- dihydropyridine-3- carboxamide | From Intermediate 26 Amidation procedure C White solid Yield: 12.0 g, 12.3% LCMS (ES⁺): 451.0 [MH]⁺ UPLC: Rt 2.83 min, 100% purity |
| 44 | 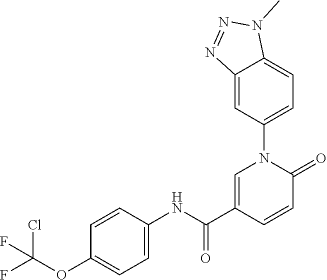 [SMILES: Cn1nnc2cc(-n3cc(C(=O)Nc4ccc(OC(F)(F)Cl)cc4)ccc3=O)ccc21] | N-[4- (Chlorodifluoro- methoxy)phenyl]-1-(1- methyl-1H-1,2,3- benzotriazol-5-yl)-6- oxo-1,6- dihydropyridine-3- carboxamide | From Intermediate 44 Amidation procedure C Yellow solid Yield: 5.0 mg, 5.1% LCMS (ES⁺): 446.0 [MH]⁺ UPLC: Rt 2.71 min, 98.1% purity |
| 45 | 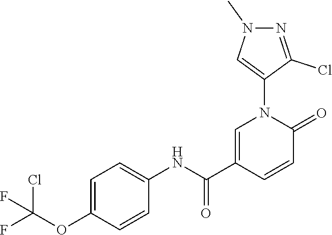 [SMILES: Cn1cc(-n2cc(C(=O)Nc3ccc(OC(F)(F)Cl)cc3)ccc2=O)c(Cl)n1] | 1-(3-Chloro-1- methyl-1H-pyrazol- 4-yl)-N-[4- (chlorodifluoro- methoxy)phenyl]-6-oxo- 1,6-dihydropyridine- 3-carboxamide | From Intermediate 45 Amidation procedure E Yellow solid Yield: 56.0 mg, 45.1% LCMS (ES⁺): 428.9 [MH]⁺ UPLC: Rt 2.75 min, 100% purity |
| 46 | 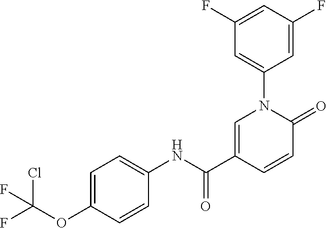 [SMILES: O=C(Nc1ccc(OC(F)(F)Cl)cc1)c1ccc(=O)n(-c2cc(F)cc(F)c2)c1] | N-[4- (Chlorodifluoro- methoxy)phenyl]-1-(3,5- difluorophenyl)-6- oxo-1,6- dihydropyridine-3- carboxamide | From Intermediate 46 Amidation procedure C White solid Yield: 54.0 mg, 19.1% LCMS (ES⁺): 427.0 [MH]⁺ UPLC: Rt 2.94 min, 99.6% purity |
| 47 | 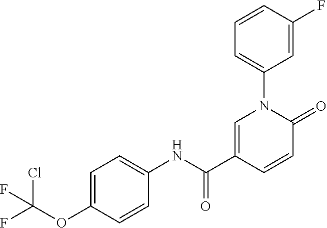 [SMILES: O=C(Nc1ccc(OC(F)(F)Cl)cc1)c1ccc(=O)n(-c2cccc(F)c2)c1] | N-[4- (Chlorodifluoro- methoxy)phenyl]-1-(3- fluorophenyl)-6-oxo- 1,6-dihydropyridine- 3-carboxamide | Amidation procedure D White solid Yield: 61.0 mg, 11.6% LCMS (ES⁺): 408.8 [MH]⁺ UPLC: Rt 2.89 min, 99.8% purity |
| 48 | 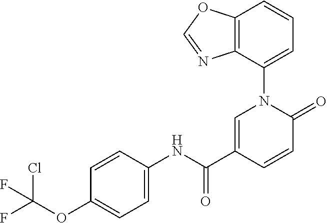 [SMILES: O=C(Nc1ccc(OC(F)(F)Cl)cc1)c1ccc(=O)n(-c2cccc3ocnc23)c1] | 1-(1,3-Benzoxazol- 4-yl)-N-[4- (chlorodifluoro- methoxy)phenyl]-6-oxo- 1,6-dihydropyridine- 3-carboxamide | From Intermediate 49 Amidation procedure E White solid Yield: 33.0 mg, 6.8% LCMS (ES⁺): 432.0 [MH]⁺ UPLC: Rt 2.55 min, 96.2% purity |
| 49 | 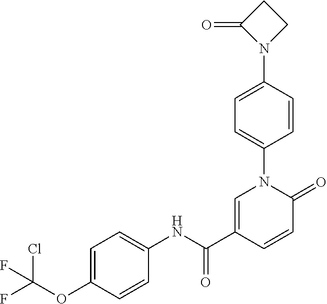 [SMILES: O=C(Nc1ccc(OC(F)(F)Cl)cc1)c1ccc(=O)n(-c2ccc(N3CCC3=O)cc2)c1] | N-[4- [Chloro(difluoro) methoxy]phenyl]-6-oxo- 1-[4-(2-oxoazetidin- 1-yl)phenyl]pyridine- 3-carboxamide | From Intermediate 47 White solid Yield 48.0 mg, 10.8% LCMS (ES⁺): 460.0 [MH]⁺ UPLC: Rt 5.78 min, 99.6% purity |

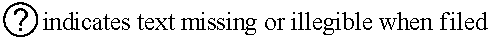

### Example 50

### 6-Oxo-1-(pyrimidin-5-yl)-N-[4-(trifluoromethoxy)phenyl]-1.6-dihydropyridine-3-carboxamide

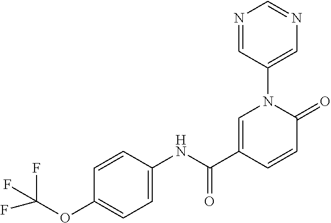 [SMILES: O=C(Nc1ccc(OC(F)(F)F)cc1)c1ccc(=O)n(-c2cncnc2)c1]

To (pyrimidin-5-yl)boronic acid (398 mg, 3.22 mmol) in MeOH (10 mL), Cu(OTf)₂ (1.17 g, 3.22 mmol) and Intermediate 54 were added sequentially, followed by pyridine (551 μL, 5.15 mmol). The RM was stirred at 25° C. for 16 h in a sealed vessel equipped with a bubble counter then the precipitate was filtered off and the filtrate was concentrated under vacuum. The residue was dissolved in EtOAc (20 mL), washed with aq ammonia (2×50 mL) and the organic layer was concentrated under vacuum. Purification by HPLC gave the title compound (253 mg, 20.9%) as a brown solid. LCMS (ES⁺): 377.0 [MH]⁺. HPLC: Rt 4.95 min, 99.7% purity.

### Examples 51-60

Examples 51-60 were prepared similarly to Example 50, by Chan-Lam coupling of Intermediates 53, 56, 57 and 60 with the appropriate aryl or heteroaryl boronate ester or boronic acid; see Table 6 below.

TABLE 6
Chan-Lam reactions

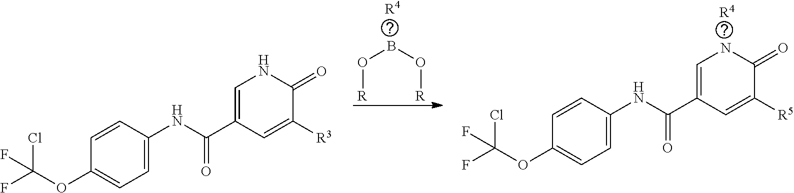

| | | | Intermediate(s) |
|---|---|---|---|
| | | | used, Form, Yield, |
| Ex. | Structure | Name | LCMS, UPLC |
| 51 | 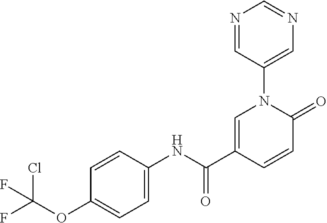 [SMILES: O=C(Nc1ccc(OC(F)(F)Cl)cc1)c1ccc(=O)n(-c2cncnc2)c1] | N-[4- (Chlorodifluoro- methoxy)phenyl]-6-oxo- 1-(pyrimidin-5-yl)- 1,6- dihydropyridine-3- carboxamide | From Intermediate 53 White solid Yield 107 mg, 13.4% LCMS (ES⁺): 393.0 [MH]⁺ UPLC: Rt 5.12 min, 98.4% purity |
| 52 | 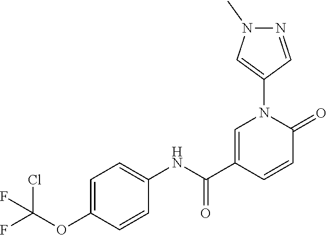 [SMILES: Cn1cc(-n2cc(C(=O)Nc3ccc(OC(F)(F)Cl)cc3)ccc2=O)cn1] | N-[4- [Chloro(difluoro) methoxy]phenyl]-1-(1- methylpyrazol-4-yl)- 6-oxo-pyridine-3- carboxamide | From Intermediate 53 White solid Yield 49.7 mg, 19.7%^{$} LCMS (ES⁺): 395.0 [MH]⁺ UPLC: Rt 5.32 min, 99.3% purity |
| 53 | 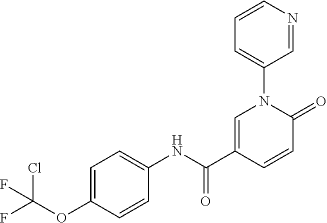 [SMILES: O=C(Nc1ccc(OC(F)(F)Cl)cc1)c1ccc(=O)n(-c2cccnc2)c1] | N-[4- [Chloro(difluoro) methoxy]phenyl]-6- oxo-1-(3- pyridyl)pyridine-3- carboxamide | From Intermediate 53 Off-white solid Yield 29.0 mg, 11.6%^{$} LCMS (ES⁺): 392.0 [MH]⁺ UPLC: Rt 4.99 min, 98.9% purity |
| 54 | 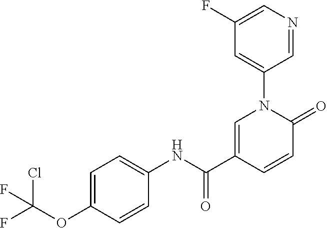 [SMILES: O=C(Nc1ccc(OC(F)(F)Cl)cc1)c1ccc(=O)n(-c2cncc(F)c2)c1] | N-[4- [Chloro(difluoro) methoxy]phenyl]-1-(5- fluoro-3-pyridyl)-6- oxo-pyridine-3- carboxamide | From Intermediate 53 White solid Yield 173 mg, 66.7%^{$} LCMS (ES⁺): 410.0 [MH]⁺ UPLC: Rt 5.55 min, 100% purity |
| 55 | 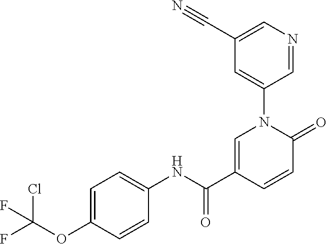 [SMILES: N#Cc1cncc(-n2cc(C(=O)Nc3ccc(OC(F)(F)Cl)cc3)ccc2=O)c1] | N-[4- [Chloro(difluoro) methoxy]phenyl]-1-(5- cyano-3-pyridyl)-6- oxo-pyridine-3- carboxamide | From Intermediate 53 White solid Yield 50.2 mg, 18.7%^{$} LCMS (ES⁺): 417.0 [MH]⁺ UPLC: Rt 5.54 min, 98.2% purity |
| 56 | 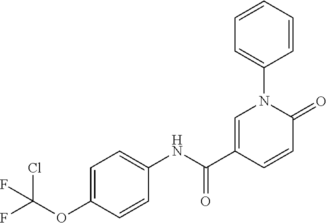 [SMILES: O=C(Nc1ccc(OC(F)(F)Cl)cc1)c1ccc(=O)n(-c2ccccc2)c1] | N-[4- [Chloro(difluoro) methoxy]phenyl]-6- oxo-1-phenyl- pyridine-3- carboxamide | From Intermediate 53 White solid Yield 83.3 mg, 22.7%^{$} LCMS (ES⁺): 391.0 [MH]⁺ UPLC: Rt 5.92 min, 99.5% purity |
| 57 | 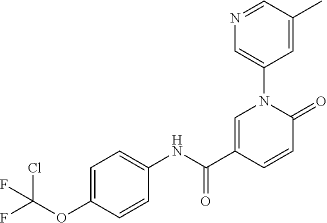 [SMILES: Cc1cncc(-n2cc(C(=O)Nc3ccc(OC(F)(F)Cl)cc3)ccc2=O)c1] | N-[4- [Chloro(difluoro) methoxy]phenyl]-1-(5- methyl-3-pyridyl)-6- oxo-pyridine-3- carboxamide | From Intermediate 53 White solid Yield 97.2 mg, 38.2%^{$} LCMS (ES⁺): 406.0 [MH]⁺ UPLC: Rt 5.09 min, 99.5% purity |
| 58 | 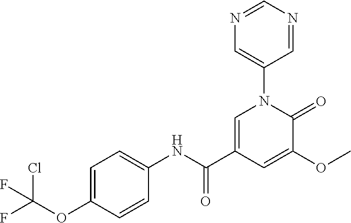 [SMILES: COc1cc(C(=O)Nc2ccc(OC(F)(F)Cl)cc2)cn(-c2cncnc2)c1=O] | N-[4- [Chloro(difluoro) methoxy]phenyl]-5- methoxy-6-oxo-1- pyrimidin-5-yl- pyridine-3- carboxamide | From Intermediate 56 White solid Yield 21.7 mg, 8.9%^{$} LCMS (ES⁺): 423.0 [MH]⁺ UPLC: Rt 5.21 min, 99.8% purity |
| 59 | 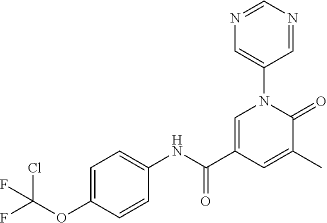 [SMILES: Cc1cc(C(=O)Nc2ccc(OC(F)(F)Cl)cc2)cn(-c2cncnc2)c1=O] | N-[4- [Chloro(difluoro) methoxy]phenyl]-5- methyl-6-oxo-1- pyrimidin-5-yl- pyridine-3- carboxamide | From Intermediate 57 White solid Yield 43.9 mg, 14.4%^{$} LCMS (ES⁺): 407.1 [MH]⁺ UPLC: Rt 5.47 min, 99.5% purity |
| 60 | 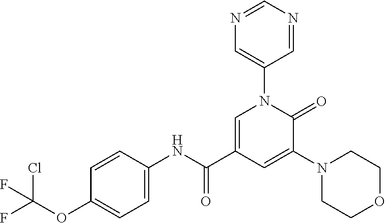 [SMILES: O=C(Nc1ccc(OC(F)(F)Cl)cc1)c1cc(N2CCOCC2)c(=O)n(-c2cncnc2)c1] | N-[4- (Chlorodifluoro- methoxy)phenyl]-5- (morpholin-4-yl)-6- oxo-1-(pyrimidin-5- yl)-1,6- dihydropyridine-3- carboxamide | From Intermediate 60 White solid Yield 73.0 mg, 24.3% LCMS (ES⁺): 478.0 [MH]⁺ UPLC: Rt 5.43 min, 99.3% purity |

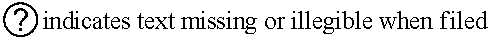

### Example 61

### N-[4-[Chloro(difluoro)methoxy]phenyl]-1-(5-methoxy-3-pyridyl)-6-oxo-pyridine-3-carboxamide

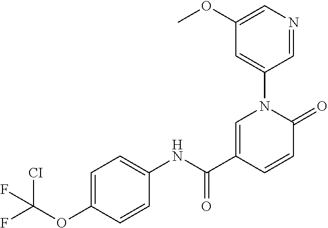

Intermediate 53 (200 mg, 97.8% pure, 622 μmol), Cs₂CO₂ (621 mg, 1.91 mmol), DMEDA (137 μL, 1.27 mmol), and 3-bromo-5-methoxypyridine (239 mg, 1.27 mmol) in 1,4-dioxane (3.0 mL) was degassed with N₂ for 5 min. CuI (60.5 mg, 318 μmol) was added, the vial was sealed and heated at 120° C. for 15 h. The RM was added to water (30 mL) and extracted with DCM (30 mL). The organic phase was dried (MgSO4) and concentrated in vacuo. The residue was purified by RP column chromatography to give the title compound (53.5 mg, 20.4%^{$}) as an off-white solid. LCMS (ES⁺): 422.0 [MH]⁺. HPLC: Rt 5.40 min, 99.8% purity.

### Examples 62-75

Examples 62-75 were prepared similarly to Example 61, by Ullmann reaction of Intermediate 53 with the appropriate aryl or heteroaryl halide; see Table 7 below.

TABLE 7
Ullmann reactions

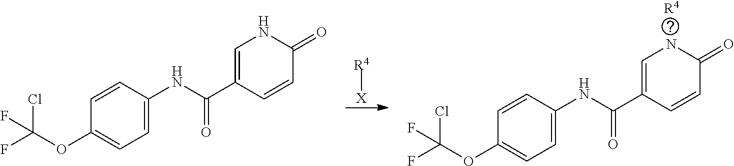

| | | | Intermediate(s) used, |
|---|---|---|---|
| | | | Form, Yield, LCMS, |
| Ex. | Structure | Name | UPLC |
| 62 | 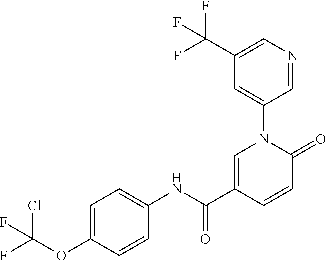 [SMILES: O=C(Nc1ccc(OC(F)(F)Cl)cc1)c1ccc(=O)n(-c2cncc(C(F)(F)F)c2)c1] | N-[4- [Chloro(difluoro)methoxy] phenyl]-6-oxo-1-[5- (trifluoromethyl)-3- pyridyl]pyridine-3- carboxamide | From Intermediate 53 Off-white solid Yield 31.2 mg, 10.5%^{$} LCMS (ES⁺): 460.0 [MH]⁺ UPLC: Rt 6.10 min, 96.2% purity |
| 63 | 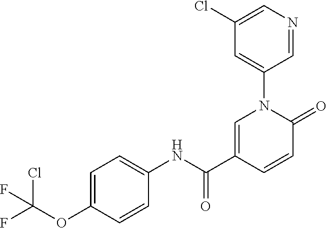 [SMILES: O=C(Nc1ccc(OC(F)(F)Cl)cc1)c1ccc(=O)n(-c2cncc(Cl)c2)c1] | N-[4- [Chloro(difluoro)methoxy] phenyl]-1-(5-chloro- 3-pyridyl)-6-oxo- pyridine-3- carboxamide | From Intermediate 53 Off-white solid Yield 24.0 mg, 18.0%^{$} LCMS (ES⁺): 426.0 [MH]⁺ UPLC: Rt 5.84 min, 99.2% purity |
| 64 | 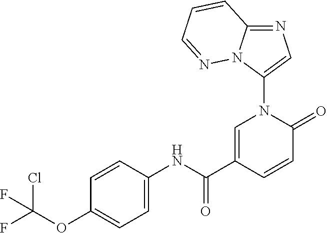 [SMILES: O=C(Nc1ccc(OC(F)(F)Cl)cc1)c1ccc(=O)n(-c2cnc3cccnn23)c1] | N-[4- (Chlorodifluoromethoxy) phenyl]-1- {imidazo[1,2- b]pyridazin-3-yl}-6- oxo-1,6- dihydropyridine-3- carboxamide | From Intermediate 53 Yellow solid Yield 14.0 mg, 4.3% LCMS (ES⁺): 432.2 [MH]⁺ UPLC: Rt 5.18 min, 96.7% purity |
| 65 | 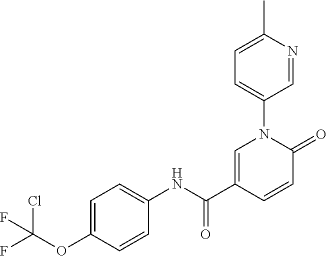 [SMILES: Cc1ccc(-n2cc(C(=O)Nc3ccc(OC(F)(F)Cl)cc3)ccc2=O)cn1] | N-[4- (Chlorodifluoromethoxy) phenyl]-6′-methyl-2- oxo-2H-[1,3′- bipyridine]-5- carboxamide | From Intermediate 53 White solid Yield 46.0 mg, 14.1% LCMS (ES⁺): 406.0 [MH]⁺ UPLC: Rt 2.42 min, 99.4% purity |
| 66 | 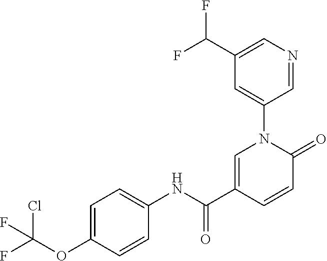 [SMILES: O=C(Nc1ccc(OC(F)(F)Cl)cc1)c1ccc(=O)n(-c2cncc(C(F)F)c2)c1] | N-[4- (Chlorodifluoromethoxy) phenyl]-5′- (difluoromethyl)-2- oxo-2H-[1,3′- bipyridine]-5- carboxamide | From Intermediate 53 White solid Yield 67.0 mg, 19.4% LCMS (ES⁺): 442.0 [MH]⁺ UPLC: Rt 2.79 min, 99.5% purity |
| 67 | 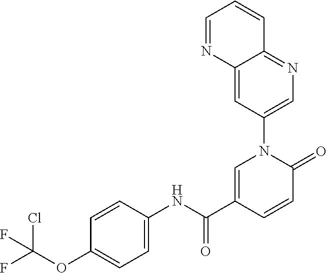 [SMILES: O=C(Nc1ccc(OC(F)(F)Cl)cc1)c1ccc(=O)n(-c2cnc3cccnc3c2)c1] | N-[4- (Chlorodifluoromethoxy) phenyl]-1-(1,5- naphthyridin-3-yl)-6- oxo-1,6- dihydropyridine-3- carboxamide | From Intermediate 53 Yellow solid Yield 23.0 mg, 7.1% LCMS (ES⁺): 443.0 [MH]⁺ UPLC: Rt 2.66 min, 100% purity |
| 68 | 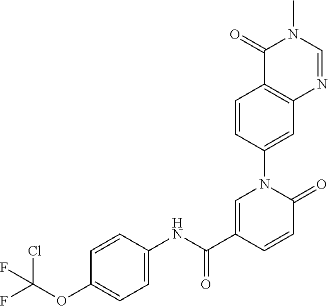 [SMILES: Cn1cnc2cc(-n3cc(C(=O)Nc4ccc(OC(F)(F)Cl)cc4)ccc3=O)ccc2c1=O] | N-[4- (Chlorodifluoromethoxy) phenyl]-1-(3-methyl- 4-oxo-3,4- dihydroquinazolin-7- yl)-6-oxo-1,6- dihydropyridine-3- carboxamide | From Intermediate 53 White solid Yield 46.0 mg, 12.4% LCMS (ES⁺): 473.2 [MH]⁺ UPLC: Rt 2.65 min, 98.2% purity |
| 69 | 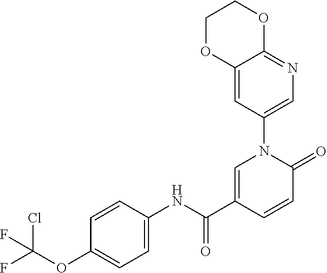 [SMILES: O=C(Nc1ccc(OC(F)(F)Cl)cc1)c1ccc(=O)n(-c2cnc3c(c2)OCCO3)c1] | N-[4- (Chlorodifluoromethoxy) phenyl]-1-{2H,3H- [1,4]dioxino[2,3- b]pyridin-7-yl}-6-oxo- 1,6-dihydropyridine-3- carboxamide | From Intermediate 53 Beige solid Yield 7.5 mg, 2.3% LCMS (ES⁺): 450.0 [MH]⁺ UPLC: Rt 2.69 min, 96.2% purity |
| 70 | 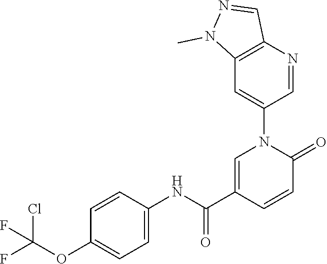 [SMILES: Cn1ncc2ncc(-n3cc(C(=O)Nc4ccc(OC(F)(F)Cl)cc4)ccc3=O)cc21] | N-[4- (Chlorodifluoromethoxy) phenyl]-1-{1-methyl- 1H-pyrazolo[4,3- b]pyridin-6-yl}-6-oxo- 1,6-dihydropyridine-3- carboxamide | From Intermediate 53 Yellow solid Yield 41.0 mg, 11.6% LCMS (ES⁺): 446.0 [MH]⁺ UPLC: Rt 2.65 min, 97.9% purity |
| 71 | 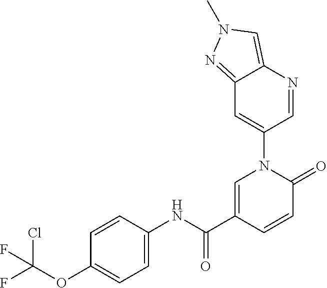 [SMILES: Cn1cc2ncc(-n3cc(C(=O)Nc4ccc(OC(F)(F)Cl)cc4)ccc3=O)cc2n1] | N-[4- (Chlorodifluoromethoxy) phenyl]-1-{2-methyl- 2H-pyrazolo[4,3- b]pyridin-6-yl}-6-oxo- 1,6-dihydropyridine-3- carboxamide | From Intermediate 53 Yellow solid Yield 23.0 mg, 9.3% LCMS (ES⁺): 446.0 [MH]⁺ UPLC: Rt 2.60 min, 98.2% purity |
| 72 | 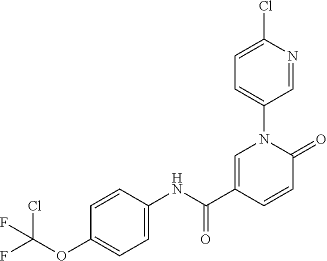 [SMILES: O=C(Nc1ccc(OC(F)(F)Cl)cc1)c1ccc(=O)n(-c2ccc(Cl)nc2)c1] | 6′-Chloro-N-[4- (chlorodifluoromethoxy) phenyl]-2-oxo-2H- [1,3′-bipyridine]-5- carboxamide | From Intermediate 53 White solid Yield 58.0 mg, 17.1% LCMS (ES⁺): 425.9 [MH]⁺ UPLC: Rt 2.82 min, 100% purity |
| 73 | 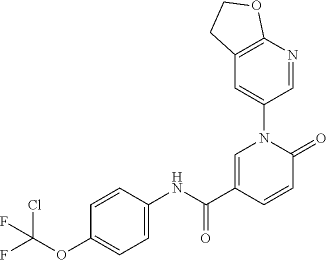 [SMILES: O=C(Nc1ccc(OC(F)(F)Cl)cc1)c1ccc(=O)n(-c2cnc3c(c2)CCO3)c1] | N-[4- (Chlorodifluoromethoxy) phenyl]-1-{2H,3H- furo[2,3-b]pyridin-5- yl}-6-oxo-1,6- dihydropyridine-3- carboxamide | From Intermediate 53 Yellow solid Yield 14.0 mg, 4.1% LCMS (ES⁺): 434.0 [MH]⁺ UPLC: Rt 2.67 min, 99.7% purity |
| 74 | 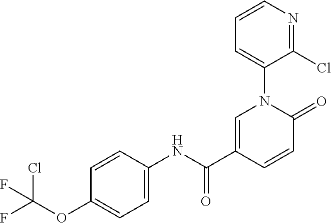 [SMILES: O=C(Nc1ccc(OC(F)(F)Cl)cc1)c1ccc(=O)n(-c2cccnc2Cl)c1] | 2′-Chloro-N-[4- (chlorodifluoromethoxy) phenyl]-2-oxo-2H- [1,3′-bipyridine]-5- carboxamide | From Intermediate 53 Yellow solid Yield 7.0 mg, 2.1% LCMS (ES⁺): 426.0 [MH]⁺ UPLC: Rt 3.03 min, 96.1% purity |
| 75 | 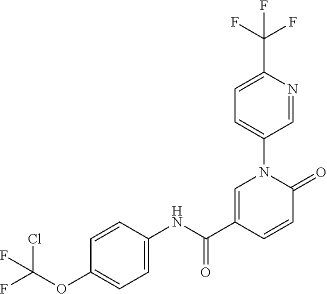 [SMILES: O=C(Nc1ccc(OC(F)(F)Cl)cc1)c1ccc(=O)n(-c2ccc(C(F)(F)F)nc2)c1] | N-[4- (Chlorodifluoromethoxy) phenyl]-2-oxo-6′- (trifluoromethyl)-2H- [1.3′-bipyridine]-5- carboxamide | From Intermediate 53 Yellow solid Yield 12.0 mg, 3.1% LCMS (ES⁺): 460.0 [MH]⁺ UPLC: Rt 2.92 min, 95.4% purity |

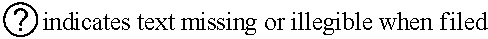

### Example 76

### N-[4-(Chlorodifluoromethoxy)phenyl]-1-methyl-6-oxo-1,6-dihydropyridine-3-carboxamide

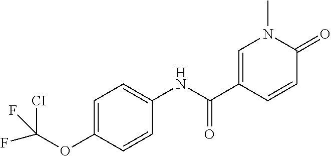

Example 76 was isolated as a byproduct from an attempted Ullman coupling between Intermediate 53 and 3-bromo-5-fluoro-4-methoxypyridine, following a procedure similar to that used in the synthesis of Example 61. Example 76 was isolated (16.0 mg, 16.3%) as a white solid. LCMS (ES⁺): 329.1 [MH]⁺. HPLC: Rt 2.55 min, 100% purity.

It is expected that Example 76 could be made similarly to Example 26 using 4-[chloro(difluoro)methoxy]aniline instead of 4-(trifluoromethoxy)aniline, following General Amidation Procedure C.

### Example 77

### N-[4-(Chlorodifluoromethoxy)phenyl]-1-(oxan-4-yl)-6-oxo-1,6-dihydropyridine-3-carboxamide

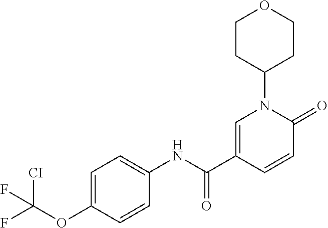

Intermediate 53 (223 mg, 0.63 mmol), Na₂CO₃ (134 mg, 1.26 mmol) and 4-iodotetrahydro-2H-pyran (164 mg, 0.76 mmol) in DMF (5.0 mL) were stirred at RT overnight then partitioned between EtOAc (30 mL) and water (10 mL). The EtOAc layer was dried over Na₂SO₄, filtered and evaporated to dryness. The residue was purified by preparative HPLC to give the title compound (12.0 mg, 5.3%) as a white solid. LCMS (ES⁺): 399.0 [MH]⁺. HPLC: Rt 6.24 min, 99.4% purity.

### Example 78

### 5-{[4-(Chlorodifluoromethoxy)phenyl]carbamoyl}-2-oxo-2H-[1.3′-bipyridin]-1′-ium-1′-olate

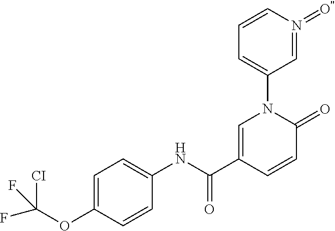

Example 53 (213 mg, 0.54 mmol) was dissolved in DCM (5.0 mL) and mCPBA (284 mg, 1.60 mmol) was added. The resulting mixture was stirred at RT for 24 h, washed with 1M aq NaOH (2×10 mL) then the organic layer was dried over Na₂SO₄, concentrated under reduced pressure and purified by prep HPLC to provide the title compound (27.0 mg, 12.3%) as a yellow solid. LCMS (ES⁺): 408.0 [MH]⁺. HPLC: Rt 2.45 min, 95.8% purity.

### Example 79

### N-[4-(Chlorodifluoromethoxy)phenyl]-5-cyano-1-(1-methyl-1H-pyrazol-4-yl)-6-oxo-1.6-dihydropyridine-3-carboxamide

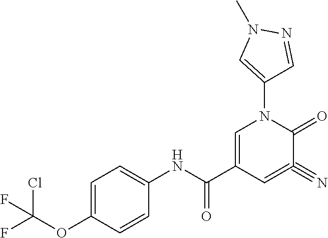

CuCN (94.0 mg, 1.05 mmol) was added to Intermediate 61 (335 mg, 0.70 mmol) then the mixture was heated under reflux for 10 h, cooled to 80° C. and poured into a solution of NaCN (214 mg, 4.37 mmol) in water (10 mL). After stirring for 1 h at RT, the mixture was extracted with EtOAc (2×25 mL) and the organic phase was washed with brine (10 mL), dried over Na₂SO₄, concentrated under reduced pressure and purified by RP HPLC to give the title compound (104 mg, 34.1%) as a beige solid. LCMS (ES⁺): 420.0 [MH]⁺. UPLC: Rt 2.75 min, 99.6% purity.

### Example 80

### N-[4-(Chlorodifluoromethoxy)phenyl]-6-oxo-5-(1H-pyrazol-3-yl)-1,6-dihydropyridine-3-carboxamide

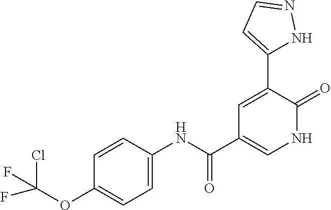 [SMILES: O=C(Nc1ccc(OC(F)(F)Cl)cc1)c1c[nH]c(=O)c(-c2ccn[nH]2)c1]

Example 80 was prepared similarly to Example 25 using Intermediate 52 instead of Intermediate 27 and following General Amidation Procedure D to give the title compound (63.0 mg, 6.6%) as a white solid. LCMS (ES⁺): 381.0 [MH]⁺. HPLC: Rt 2.51 min, 99.2% purity.

### Example 81

### N-[4-[Chloro(difluoro)methoxy]phenyl]-1-methyl-6-oxo-5-(3-pyridyl)pyridine-3-carboxamide

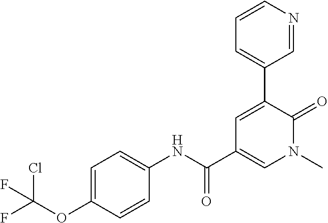 [SMILES: Cn1cc(C(=O)Nc2ccc(OC(F)(F)Cl)cc2)cc(-c2cccnc2)c1=O]

Intermediate 64 (100 mg, 94.2% purity, 231 μmol), pyridin-3-ylboronic acid (56.8 mg, 462 μmol), K₂CO₃ (95.8 mg, 693 μmol), Pd(OAc)₂ (10.4 mg, 46.2 μmol) and PPh₃ (30.3 mg, 116 μmol) in dioxane (1.0 mL) were heated in a sealed tube at 110° C. under N₂ for 18 h, then the RM was purified by column chromatography to give the title compound (48.3 mg, 50.8%^{$}) as a beige solid. LCMS (ES⁺): 406.0 [MH]⁺. UPLC: Rt 4.54 min, 98.7% purity.

### Examples 82-89

Examples 82-89 were prepared similarly to Example 81, by Suzuki reaction of Intermediates 61 and 64 with the appropriate aryl or heteroaryl boronic acid or boronate ester; see Table 8 below.

TABLE 8
Suzuki reactions

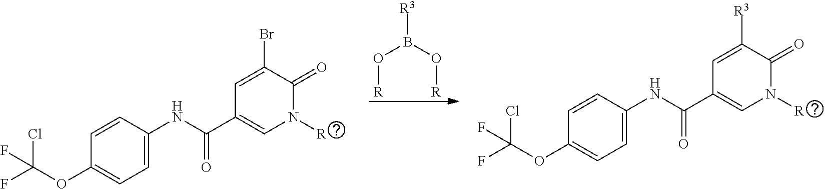

| | | | Intermediate(s) |
|---|---|---|---|
| | | | used, Form, Yield, |
| Ex. | Structure | Name | LCMS, UPLC |
| 82 | 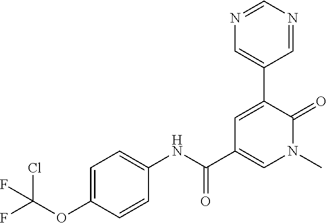 [SMILES: Cn1cc(C(=O)Nc2ccc(OC(F)(F)Cl)cc2)cc(-c2cncnc2)c1=O] | N-[4- (Chlorodifluoromethoxy) phenyl]-1-methyl- 6-oxo-5-(pyrimidin-5- yl)-1,6- dihydropyridine-3- carboxamide | From Intermediate 64 Yellow solid Yield 57.0 mg, 28.6% LCMS (ES⁺): 407.0 [MH]⁺ UPLC: Rt 5.29 min, 99.2% purity |
| 83 | 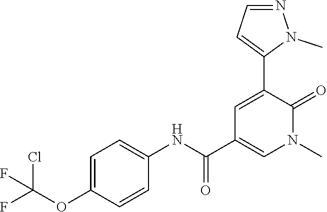 [SMILES: Cn1nccc1-c1cc(C(=O)Nc2ccc(OC(F)(F)Cl)cc2)cn(C)c1=O] | N-[4- (Chlorodifluoromethoxy) phenyl]-1-methyl- 5-(1-methyl-1H- pyrazol-5-yl)-6-oxo- 1,6-dihydropyridine- 3-carboxamide | From Intermediate 64 White solid Yield 54.0 mg, 25.3% LCMS (ES⁺): 409.0 [MH]⁺ UPLC: Rt 5.44 min, 99.4% purity |
| 84 | 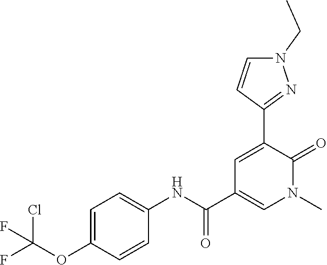 [SMILES: CCn1ccc(-c2cc(C(=O)Nc3ccc(OC(F)(F)Cl)cc3)cn(C)c2=O)n1] | N-[4- (Chlorodifluoromethoxy) phenyl]-5-(1-ethyl- 1H-pyrazol-3-yl)-1- methyl-6-oxo-1,6- dihydropyridine-3- carboxamide | From Intermediate 64 White solid Yield 47.0 mg, 32.9% LCMS (ES⁺): 423.2 [MH]⁺ UPLC: Rt 5.81 min, 99.7% purity |
| 85 | 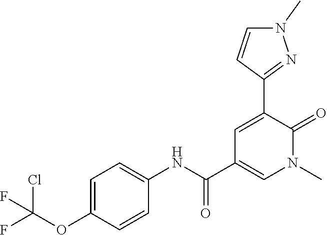 [SMILES: Cn1ccc(-c2cc(C(=O)Nc3ccc(OC(F)(F)Cl)cc3)cn(C)c2=O)n1] | N-[4- (Chlorodifluoromethoxy) phenyl]-1-methyl- 5-(1-methyl-1H- pyrazol-3-yl)-6-oxo- 1,6-dihydropyridine- 3-carboxamide | From Intermediate 64 Yellow solid Yield 53.0 mg, 25.4% LCMS (ES⁺): 409.2 [MH]⁺ UPLC: Rt 5.50 min, 98.8% purity |
| 86 | 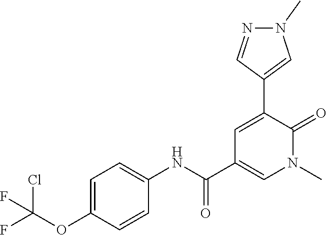 [SMILES: Cn1cc(-c2cc(C(=O)Nc3ccc(OC(F)(F)Cl)cc3)cn(C)c2=O)cn1] | N-[4- [Chloro(difluoro)methoxy] phenyl]-1- methyl-5-(1- methylpyrazol-4-yl)- 6-oxo-pyridine-3- carboxamide | From Intermediate 64 White solid Yield 34.5 mg, 36.3%^{$} LCMS (ES⁺): 409.0 [MH]⁺ UPLC: Rt 5.51 min, 99.5% purity |
| 87 | 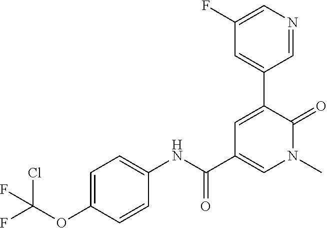 [SMILES: Cn1cc(C(=O)Nc2ccc(OC(F)(F)Cl)cc2)cc(-c2cncc(F)c2)c1=O] | N-[4- [Chloro(difluoro)methoxy] phenyl]-5-(5- fluoro-3-pyridyl)-1- methyl-6-oxo- pyridine-3- carboxamide | From Intermediate 64 Beige solid Yield 14.5 mg, 14.7%^{$} LCMS (ES⁺): 424.0 [MH]⁺ UPLC: Rt 5.57 min, 99.1% purity |
| 88 | 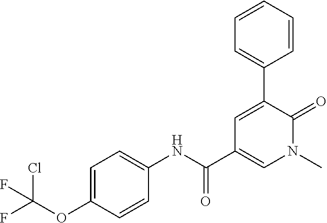 [SMILES: Cn1cc(C(=O)Nc2ccc(OC(F)(F)Cl)cc2)cc(-c2ccccc2)c1=O] | N-[4- [Chloro(difluoro)methoxy] phenyl]-1- methyl-6-oxo-5- phenyl-pyridine-3- carboxamide | From Intermediate 64 White solid Yield 67.7 mg, 71.8%^{$} LCMS (ES⁺): 405.0 [MH]⁺ UPLC: Rt 6.32 min, 99.2% purity |
| 89 | 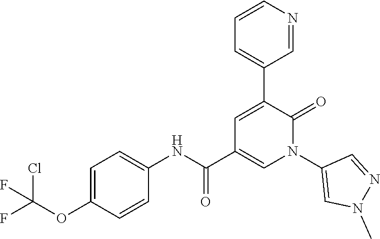 [SMILES: Cn1cc(-n2cc(C(=O)Nc3ccc(OC(F)(F)Cl)cc3)cc(-c3cccnc3)c2=O)cn1] | N-[4- (Chlorodifluoromethoxy) phenyl]-1-(1- methyl-1H-pyrazol-4- yl)-2-oxo-1,2- dihydro-[3,3′- bipyridine]-5- carboxamide | From Intermediate 61 Beige solid Yield 12.0 mg, 19.3% LCMS (ES⁺): 472.0 [MH]⁺ UPLC: Rt 2.40 min, 96.8% purity |

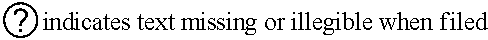

### Example 90

### N-[4-[Chloro(difluoro)methoxy]phenyl]-1-methyl-6-oxo-5-pyrazol-1-yl-pyridine-3-carboxamide

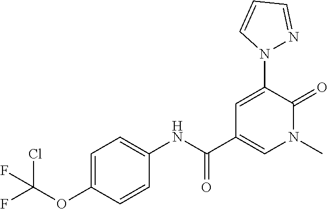 [SMILES: Cn1cc(C(=O)Nc2ccc(OC(F)(F)Cl)cc2)cc(-n2cccn2)c1=O]

To a mixture of Intermediate 64 (100 mg, 94.2% purity, 231 μmol), CuI (8.80 mg, 46.2 μmol) and K₂CO₃ (95.8 mg, 693 μmol) in toluene (4.0 mL) was added trans N,N-dimethylcyclohexane-1,2-diamine (7.29 μL, 46.2 μmol) and pyrazole (31.5 mg, 462 μmol), then the RM heated at 110° C. under nitrogen for 66 h. The RM was diluted with DCM (5.0 mL), filtered, then purified by column chromatography and RP HPLC to give the title compound (21.6 mg, 23.5%^{$}) as a white solid. LCMS (ES⁺): 394.8 [MH]⁺. UPLC: Rt 5.81 min, 99.4% purity.

### Example 91

### N-[4-[Chloro(difluoro)methoxy]phenyl]-5-imidazol-1-yl-1-methyl-6-oxo-pyridine-3-carboxamide

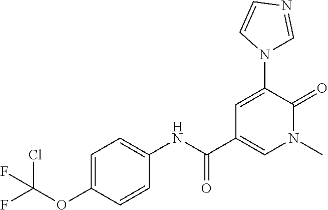 [SMILES: Cn1cc(C(=O)Nc2ccc(OC(F)(F)Cl)cc2)cc(-n2ccnc2)c1=O]

Example 91 was prepared similarly to Example 90 from Intermediate 64, using imidazole instead of pyrazole, to give the title compound (8.25 mg, 5.9%^{$}) as a beige solid. LCMS (ES⁺): 395.0 [MH]⁺. UPLC: Rt 4.53 min, 97.7% purity.

### Example 92

### N-[4-(Chlorodifluoromethoxy)phenyl]-2-oxo-6-(1H-pyrazol-5-yl)-1,2-dihydropyridine-4-carboxamide

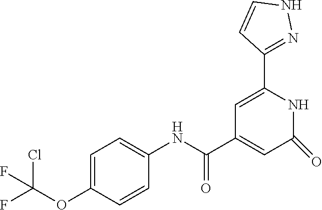 [SMILES: O=C(Nc1ccc(OC(F)(F)Cl)cc1)c1cc(-c2cc[nH]n2)[nH]c(=O)c1]

Intermediate 59 (201 mg, 554 μmol), 3-(tetramethyl-1,3,2-dioxaborolan-2-yl)-1H-pyrazole (140 mg, 720 μmol), K₂CO₃ (305 mg, 2.22 mmol) and Pd(dppf)Cl₂.DCM (23.0 mg, 28.0 μmol) in dioxane-water (10 mL, 1:1, v/v) was heated to 90° C. for 12 h. The RM was mixed with water (5.0 mL), extracted with EtOAc (2×10 mL) and concentrated in vacuo. The residue was treated with conc HCl (5.0 mL) and THF (5.0 mL) and stirred at 35-40° C. for 1 h. After cooling to RT the RM was extracted with EtOAc (2×10 mL) and the combined organic extracts were dried over MgSO4, concentrated in vacuo and purified by RP HPLC to give the title compound (12.0 mg, 5.7%) as a white solid. LCMS (ES⁺): 381.2 [MH]⁺. UPLC: Rt 2.56 min, 99.2% purity.

### Example 93

### 2-Oxo-6-(pyrimidin-5-yl)-N-[4-(trifluoromethoxy)phenyl]-1,2-dihydropyridine-4-carboxamide

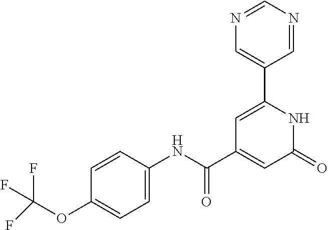 [SMILES: O=C(Nc1ccc(OC(F)(F)F)cc1)c1cc(-c2cncnc2)[nH]c(=O)c1]

Intermediate 66 (313 mg, 0.77 mmol) was dissolved in AcOH (2.0 mL), conc HCl (2.0 mL) was added and the RM was stirred for 12 h at 50° C. then concentrated in vacuo and purified by RP HPLC to give the title compound (14.0 mg, 3.1%) as an off-white solid. LCMS (ES⁺): 377.2 [MH]⁺. HPLC: Rt 1.26 min, 100% purity.

### Example 94

### N-[4-(Chlorodifluoromethoxy)phenyl]-1-methyl-6-(1-methyl-1H-pyrazol-4-yl)-2-oxo-1,2-dihydropyridine-4-carboxamide

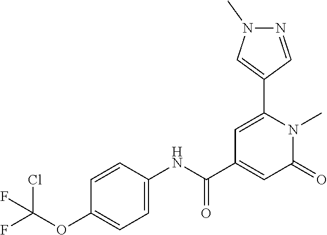 [SMILES: Cn1cc(-c2cc(C(=O)Nc3ccc(OC(F)(F)Cl)cc3)cc(=O)n2C)cn1]

A mixture of Intermediate 67 (152 mg, 0.41 mmol), 1-methyl-4-(4,4,5,5-tetramethyl-1,3,2-dioxaborolan-2-yl)-1H-pyrazole (134 mg, 0.62 mmol), 30% aq K₂CO₃ (284 mg, 2.07 mmol) and Pd(dppf)Cl₂DCM (9.1 mg, 12.4 μmol) was evacuated and backfilled with argon three times. Dioxane (2.0 mL) was added and the RM was stirred at 100° C. overnight then cooled to RT, the organic layer was separated, dried under Na2SO₄ and evaporated. The crude product was purified by RP HPLC (water/MeCN) to give the title compound (25.0 mg, 15.2%) as a white solid. LCMS (ES⁺): 409.2 [MH]+, Rt 5.40 min, 99.4% purity.

### REFERENCE COMPOUNDS

### Reference Example 1

### N-[4-[Chloro(difluoro)methoxy]phenyl]-6-[(3R)-3-hydroxypyrrolidin-1-yl]-5-(1H-pyrazol-5-yl)pyridine-3-carboxamide

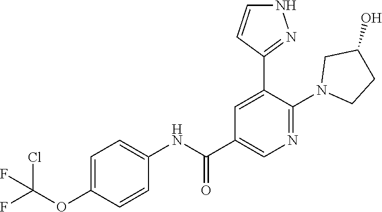 [SMILES: O=C(Nc1ccc(OC(F)(F)Cl)cc1)c1cnc(N2CC[C@@H](O)C2)c(-c2cc[nH]n2)c1]

Asciminib was purchased from Med Chem Express (CAS: 1492952-76-7) and used as received.

### Reference Example 2

### N-[4-[Chloro(difluoro)methoxy]phenyl]-3-pyrimidin-5-ylbenzamide

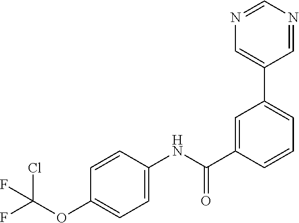 [SMILES: O=C(Nc1ccc(OC(F)(F)Cl)cc1)c1cccc(-c2cncnc2)c1]

Intermediate 68 (250 mg, 0.59 mmol), pyrimidine-5-boronic acid pinacol ester (304 mg, 1.48 mmol) and Pd(dppf)Cl₂.DCM (48.2 mg, 0.06 mmol) in a mixture of 1,4-dioxane (15 mL) and 2M aq Na₂CO₃ (5.0 mL, 10.0 mmol) were heated by microwave at 130° C. for 30 min. The RM was partitioned between EtOAc (20 mL) and water (20 mL), and the separated aqueous layer was extracted with EtOAc (20 mL). The combined organic layers were washed with brine (20 mL), dried (MgSO4), concentrated in vacuo and the residue purified by column chromatography to give the title compound (174 mg, 78.5%) as a grey solid. LCMS (ES⁺): 376.0 [MH]⁺. Rt 5.96 min, 100% purity.

### Reference Example 3

### 4-Methyl-3-(3-pyridyl)-N-[4-(trifluoromethoxy)phenyl]benzamide

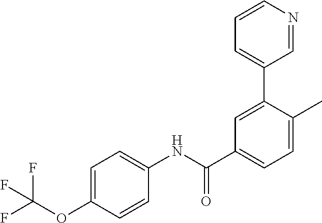 [SMILES: Cc1ccc(C(=O)Nc2ccc(OC(F)(F)F)cc2)cc1-c1cccnc1]

Intermediate 69 (2.40 g, 5.70 mmol), pyridin-3-ylboronic acid (1.05 g, 8.55 mmol),

Na2CO3 (1.81 g, 17.1 mmol) and Pd(PPh3)₄ (400 mg, 570 μmol) were dissolved in EtOH/DME/water (13.5 mL, 1.5:10:2). The reaction was heated using a microwave reactor to 130° C. for 30 min. The solvents were removed in vacuo and the residue purified by column chromatography and RP HPLC to give the title compound (320 mg, 15.1%^{$}) as a white solid. LCMS (ES⁺): 373.1 [MH]⁺. UPLC:

Rt 4.93 min, 99.1% purity.

### Reference Example 4

### 5-(3-Pyridyl)-N-[4-(trifluoromethoxy)phenyl]pyridine-3-carboxamide

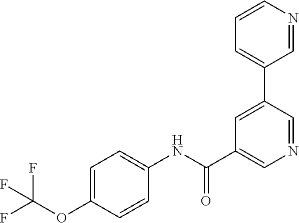 [SMILES: O=C(Nc1ccc(OC(F)(F)F)cc1)c1cncc(-c2cccnc2)c1]

Reference Example 4 was synthesised similarly to General Amidation Procedure C, using Intermediate 71 and 4-(trifluoromethoxy)aniline, to give the title compound (104 mg, 58.1%^{$}) as a white solid. LCMS (ES⁺): 360.1 [MH]⁺. UPLC: Rt 4.44 min, 100% purity.

### Reference Example 5

### 2-Oxo-1-(pyrimidin-5-yl)-N-[4-(trifluoromethoxy)phenyl]-1,2-dihydropyridine-3-carboxamide

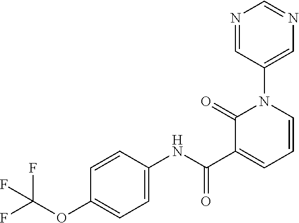 [SMILES: O=C(Nc1ccc(OC(F)(F)F)cc1)c1cccn(-c2cncnc2)c1=O]

To Intermediate 58 (483 mg, 1.62 mmol), (pyrimidin-5-yl)boronic acid (206 mg, 1.62 mmol) and Cu(OTf)₂ (604 mg, 1.62 mmol) in MeOH (10 mL) was added pyridine (0.27 mL, 2.56 mmol), then the mixture stirred at 25° C. for 16 h in a sealed vessel with a bubble counter. The precipitate was filtered off and the filtrate was concentrated under vacuum. The residue was dissolved in EtOAc (20 mL), washed with aq ammonia (2×50 mL) then then organic layer was dried over Na2SO₄, concentrated under vacuum and purified by HPLC to give the title compound (33.0 mg, 5.2%) as a beige solid. LCMS (ES⁺): 377.0 [MH]⁺. Rt 2.67 min, 99.6% purity.

High Resolution Mass Spectrometry Data

| | | | | | MIM | MIM |
|---|---|---|---|---|---|---|
| | | | | | calculated | calculated |
| | | | positive | negative | from | from |
| Example | MWt | MIM | ion m/z | ion m/z | positive ion* | negative ion* |
| 1 | 438.81 | 438.09 | 439.097 | 437.0827 | 438.0897 | 438.09 |
| 2 | 411.38 | 411.14 | 412.147 | 410.3127 | 411.1397 | 411.32 |
| 3 | 394.76 | 394.06 | 395.071 | 393.057 | 394.0637 | 394.0643 |
| 4 | 384.76 | 384.07 | 385.0755 | 383.0614 | 384.0682 | 384.0687 |
| 5 | 380.73 | 380.05 | 381.0554 | 379.0412 | 380.0481 | 380.0485 |
| 6 | 384.76 | 384.07 | 385.0755 | 383.0612 | 384.0682 | 384.0685 |
| 7 | 430.74 | 430.05 | 431.0522 | 429.0378 | 430.0449 | 430.0451 |
| 8 | 397.78 | 397.01 | 398.0169 | 396.0024 | 397.0096 | 397.0097 |
| 9 | 405.78 | 405.07 | 406.0756 | 404.0613 | 405.0683 | 405.0686 |
| 10 | 452.8 | 452.07 | 453.0762 | 451.0621 | 452.0689 | 452.0694 |
| 11 | 397.78 | 397.01 | 398.0164 | 396.0022 | 397.0091 | 397.0095 |
| 12 | 422.77 | 422.06 | 423.0657 | 421.0517 | 422.0584 | 422.059 |
| 13 | 421.78 | 421.06 | 422.0707 | 420.0565 | 421.0634 | 421.0638 |
| 14 | 422.81 | 422.1 | 423.1035 | 421.0887 | 422.0962 | 422.096 |
| 15 | 420.8 | 420.08 | 421.0862 | 419.0723 | 420.0789 | 420.0796 |
| 16 | 397.8 | 397.1 | 398.1076 | 396.093 | 397.1003 | 397.1003 |
| 17 | 397.8 | 397.1 | 398.1072 | 396.0928 | 397.0999 | 397.1001 |
| 18 | 408.79 | 408.08 | 409.0867 | 407.0725 | 408.0794 | 408.0798 |
| 19 | 434.82 | 434.1 | 435.102 | 433.0878 | 434.0947 | 434.0951 |
| 20 | 415.78 | 415.05 | 416.06 | 414.0459 | 415.0527 | 415.0532 |
| 21 | 434.82 | 434.1 | 435.1024 | 433.0878 | 434.0951 | 434.0951 |
| 22 | 438.81 | 438.09 | 439.0973 | 437.0828 | 438.09 | 438.0901 |
| 23 | 436.84 | 436.11 | 437.1181 | 435.1034 | 436.1108 | 436.1107 |
| 24 | 426.75 | 426.04 | 427.0462 | 425.0318 | 426.0389 | 426.0391 |
| 25 | 394.76 | 394.06 | 395.0711 | 393.057 | 394.0638 | 394.0643 |
| 27 | 430.74 | 430.05 | 431.0525 | 429.0378 | 430.0452 | 430.0451 |
| 28 | 442.8 | 442.06 | 443.071 | 441.0566 | 442.0637 | 442.0639 |
| 29 | 420.79 | 420.07 | 421.0751 | 419.0613 | 420.0678 | 420.0686 |
| 30 | 429.2 | 428.03 | 429.0324 | 427.0176 | 428.0251 | 428.0249 |
| 32 | 450.82 | 450.08 | 451.0871 | 449.0723 | 450.0798 | 450.0796 |
| 33 | 446.83 | 446.08 | 447.0911 | 445.0767 | 446.0838 | 446.084 |
| 34 | 432.8 | 432.07 | 433.0754 | 431.061 | 432.0681 | 432.0683 |
| 35 | 420.79 | 420.07 | 421.0754 | 419.0613 | 420.0681 | 420.0686 |
| 36 | 448.8 | 448.06 | 449.0703 | 447.056 | 448.063 | 448.0633 |
| 40 | 446.83 | 446.08 | 447.0912 | 445.0766 | 446.0839 | 446.0839 |
| 41 | 446.83 | 446.08 | 447.0926 | 455.0769 | 446.0853 | 456.0842 |
| 42 | 408.76 | 408.05 | 409.0557 | 407.0412 | 408.0484 | 408.0485 |
| 43 | 450.82 | 450.08 | 451.0859 | 449.0715 | 450.0786 | 450.0788 |
| 45 | 429.2 | 428.02 | 429.0323 | 427.0178 | 428.025 | 428.0251 |
| 46 | 426.74 | 426.03 | 427.0459 | 425.032 | 426.0386 | 426.0393 |
| 47 | 408.76 | 408.05 | 409.0558 | 407.0413 | 408.0485 | 408.0486 |
| 48 | 431.78 | 431.05 | 432.0552 | 430.0408 | 431.0479 | 431.0481 |
| 49 | 459.83 | 459.08 | 460.0863 | 458.0721 | 459.079 | 459.0794 |
| 50 | 376.29 | 376.08 | 377.0849 | 375.0708 | 376.0776 | 376.0781 |
| 51 | 392.74 | 392.05 | 393.0554 | 391.0414 | 392.0481 | 392.0487 |
| 52 | 394.76 | 394.06 | 395.0714 | 393.0571 | 394.0641 | 394.0644 |
| 53 | 391.76 | 391.05 | 392.0599 | 390.0461 | 391.0526 | 391.0534 |
| 54 | 409.75 | 409.04 | 410.0508 | 408.0364 | 409.0435 | 409.0437 |
| 55 | 416.77 | 416.05 | 417.0554 | 415.0412 | 416.0481 | 416.0485 |
| 56 | 390.77 | 390.06 | 391.0648 | 389.0509 | 390.0575 | 390.0582 |
| 57 | 405.78 | 405.07 | 406.0756 | 404.0613 | 405.0683 | 405.0686 |
| 58 | 422.77 | 422.06 | 423.0658 | 421.0517 | 422.0585 | 422.059 |
| 59 | 406.77 | 406.06 | 407.0712 | 405.0566 | 406.0639 | 406.0639 |
| 60 | 477.85 | 477.1 | 478.1082 | 476.0936 | 477.1009 | 477.1009 |
| 61 | 421.78 | 421.06 | 422.0706 | 420.0564 | 421.0633 | 421.0637 |
| 62 | 459.75 | 459.04 | 460.0474 | 458.0333 | 459.0401 | 459.0406 |
| 63 | 426.2 | 425.01 | 426.0212 | 424.0071 | 425.0139 | 425.0144 |
| 64 | 431.78 | 431.06 | 432.0663 | 430.0519 | 431.059 | 431.0592 |
| 65 | 405.78 | 405.07 | 406.0758 | 404.0614 | 405.0685 | 405.0687 |
| 66 | 441.76 | 441.05 | 442.0582 | 440.043 | 441.0509 | 441.0503 |
| 67 | 442.8 | 442.06 | 443.071 | 441.0566 | 442.0637 | 442.0639 |
| 68 | 472.83 | 472.07 | 473.0816 | 471.0671 | 472.0743 | 472.0744 |
| 69 | 449.79 | 449.05 | 450.0655 | 448.0513 | 449.0582 | 449.0586 |
| 70 | 445.81 | 445.07 | 446.082 | 444.0675 | 445.0747 | 445.0748 |
| 71 | 445.81 | 445.07 | 446.082 | 444.0677 | 445.0747 | 445.075 |
| 72 | 426.2 | 425.01 | 426.0209 | 424.0069 | 425.0136 | 425.0142 |
| 73 | 433.79 | 433.06 | 434.0707 | 432.0563 | 433.0634 | 433.0636 |
| 74 | 392.4 | 392.14 | 393.152 | 391.139 | 392.1447 | 392.1463 |
| 75 | 459.75 | 459.04 | 460.0476 | 458.0332 | 459.0403 | 459.0405 |
| 76 | 328.69 | 328.04 | 329.0494 | 327.0353 | 328.0421 | 328.0426 |
| 77 | 398.79 | 398.08 | 399.0912 | 397.0769 | 398.0839 | 398.0842 |
| 78 | 407.76 | 407.05 | 408.0549 | 406.0407 | 407.0476 | 407.048 |
| 79 | 419.77 | 419.06 | 420.066 | 418.0518 | 419.0587 | 419.0591 |
| 80 | 380.73 | 380.05 | 381.0553 | 379.0411 | 380.048 | 380.0484 |
| 81 | 405.78 | 405.07 | 406.0759 | 404.0616 | 405.0686 | 405.0689 |
| 82 | 406.77 | 406.06 | 407.0708 | 405.0568 | 406.0635 | 406.0641 |
| 83 | 408.79 | 408.08 | 409.0868 | 407.0725 | 408.0795 | 408.0798 |
| 84 | 422.81 | 422.1 | 423.1021 | 421.0883 | 422.0948 | 422.0956 |
| 85 | 408.79 | 408.08 | 409.0866 | 407.0724 | 408.0793 | 408.0797 |
| 86 | 408.79 | 408.08 | 409.0872 | 407.0734 | 408.0799 | 408.0807 |
| 87 | 423.77 | 423.06 | 424.0662 | 422.0522 | 423.0589 | 423.0595 |
| 88 | 404.79 | 404.07 | 405.0804 | 403.0663 | 404.0731 | 404.0736 |
| 89 | 471.84 | 471.09 | 472.0977 | 470.0831 | 471.0904 | 471.0904 |
| 90 | 394.76 | 394.06 | 395.0712 | 393.0571 | 394.0639 | 394.0644 |
| 91 | 394.76 | 394.06 | 395.0712 | 393.0571 | 394.0639 | 394.0644 |
| 92 | 380.73 | 380.05 | 381.0555 | 379.0412 | 380.0482 | 380.0485 |
| 93 | 376.29 | 376.08 | 377.0851 | 357.0708 | 376.0778 | 358.0781 |
| 94 | 408.79 | 408.08 | 409.0867 | 407.0725 | 408.0794 | 408.0798 |
| Reference | 449.84 | 449.11 | 450.1137 | 448.0992 | 449.1064 | 449.1065 |
| Example 1 | | | | | | |
| Reference | 375.76 | 375.06 | 376.0654 | 374.0511 | 375.0581 | 375.0584 |
| Example 2 | | | | | | |
| Reference | 372.34 | 372.11 | 373.1149 | 371.1011 | 372.1076 | 372.1084 |
| Example 3 | | | | | | |
| Reference | 359.3 | 359.09 | 360.0947 | 358.0808 | 359.0874 | 359.0881 |
| Example 4 | | | | | | |
| Reference | 376.29 | 376.08 | 377.0852 | 375.0714 | 376.0779 | 376.0787 |
| Example 5 | | | | | | |

### Biological Data

### Ba/F3 CellTiter-Glo Assay

The CellTiter-Glo luminescent cell viability assay is a homogeneous method of determining the number of viable cells in culture based on quantification of the ATP present. Briefly, IL-3 dependent Ba/F3 cells are modified to express BCR-ABL. Activity of the transformed kinase overrides IL3 dependency for cellular proliferation and survival. Test compounds that specifically inhibit kinase activity lead to programmed cell death which can be measured through the addition of CellTiter-Glo reagent. In this assay Ba/F3 cells expressing BCR-ABL (Advanced Cellular Dynamics) or parental Ba/F3 (control) cells were prepared at 5×104/mL in RPMI 1640 containing 10% FBS, 1× Glutamax and 750 ng/mL puromycin. Test compounds were dispensed into 384 well plates using the Tecan D300e at a top final assay concentration of 10 μM with dosing normalised to 0.1% DMSO in 50 μL volume. 50 μL cells were added to each well of the prepared 384 well plates and the plates spun at 1000 rpm for 1 min prior to incubation at 37° C., 5% CO₂ for 48 h. After 48 h 15 μL CellTiterGlo reagent was added to each well in the plate. Following a 60 min incubation at RT luminescence was read on the Pherastar FS reader.

The exemplified compounds of the invention were tested in the Ba/F3 CellTiter-Glo Assay and the IC₅₀ data is shown in Table 9. All of the exemplified compounds of the invention had an IC₅₀ value of 1000 nM or less. This data shows that the compounds of the invention can inhibit c-Abl.

Reference Example 5 had an IC₅₀ value of >10 μM and is therefore inactive against c-Abl. Without wishing to be bound by theory, the pyridone C═O bond in Reference Example 5 may sterically clash with the amide group, inducing an unfavourable twist in the amide-pyridone bond so that the two moieties are no longer co-planar. This may disrupt the edge-to-face pi-stacking interaction between the pyridone ring and the Tyr454 residue of c-Abl. Furthermore, the pyridone C═O bond may form a 6-membered ring through a intramolecular hydrogen bond with the NH of the amide group, which prevents the NH from forming a potentially crucial hydrogen bond to a water molecule within the active site of c-Abl, and may even displace said water molecule. The pyridone regioisomers in the compounds of the invention do not suffer these drawbacks and therefore exhibit much improved inhibition of c-Abl.

TABLE 9
Bcr-Abl IC₅₀ (A: 1000-151 nM, B: 150-31 nM, C: ≤30 nM)

| Ex. | IC₅₀ |
|---|---|
| Ref. ex. 1 | C |
| Ref. ex. 2 | C |
| Ref. ex. 3 | B |
| Ref. ex. 4 | B |
| Ref. ex. 5 | >10 μM |
| 1 | A |
| 2 | A |
| 3 | B |
| 4 | A |
| 5 | B |
| 6 | A |
| 7 | A |
| 8 | A |
| 9 | B |
| 10 | B |
| 11 | C |
| 12 | B |
| 13 | B |
| 14 | A |
| 15 | B |
| 16 | A |
| 17 | A |
| 18 | C |
| 19 | A |
| 20 | B |
| 21 | A |
| 22 | A |
| 23 | A |
| 24 | C |
| 25 | B |
| 26 | A |
| 27 | B |
| 28 | A |
| 29 | B |
| 30 | C |
| 31 | B |
| 32 | A |
| 33 | B |
| 34 | B |
| 35 | C |
| 36 | B |
| 37 | C |
| 38 | C |
| 39 | B |
| 40 | B |
| 41 | A |
| 42 | C |
| 43 | A |
| 44 | C |
| 45 | A |
| 46 | C |
| 47 | C |
| 48 | B |
| 49 | B |
| 50 | B |
| 51 | C |
| 52 | B |
| 53 | C |
| 54 | C |
| 55 | B |
| 56 | C |
| 57 | B |
| 58 | B |
| 59 | B |
| 60 | B |
| 61 | C |
| 62 | A |
| 63 | C |
| 64 | B |
| 65 | C |
| 66 | B |
| 67 | C |
| 68 | C |
| 69 | C |
| 70 | B |
| 71 | C |
| 72 | C |
| 73 | C |
| 74 | A |
| 75 | B |
| 76 | A |
| 77 | A |
| 78 | A |
| 79 | A |
| 80 | A |
| 81 | B |
| 82 | B |
| 83 | B |
| 84 | A |
| 85 | A |
| 86 | B |
| 87 | C |
| 88 | B |
| 89 | C |
| 90 | A |
| 91 | B |
| 92 | A |
| 93 | A |
| 94 | B |

### Determination of Pharmacokinetic Profile and Brain Penetrance in Rat

Male Sprague Dawley Rats 300-350 g (Charles River, UK) were group housed, n=3, under a 12 h light/dark cycle with food and water available ad libitum. At 17:00 on the day prior to dosing all food was removed. On the day of dosing animals were weighed, tail marked and dosed via oral gavage with compound at 3 mg/kg in a volume of 5 mL/kg. Animals were culled at 30 min, 1 h and 4 h post dose via intra-peritoneal administration of pentobarbital. Post mortem blood was withdrawn via cardiac puncture, and briefly stored in K2 EDTA blood tubes on ice before being spun at 14,000 g for 4 min at 4° C. Plasma was withdrawn into a 96 well plate, placed on dry ice and stored at −80° C. Brains were quickly dissected and placed on dry ice before storage at −80° C. Bioanalysis of plasma and brain samples is performed as detailed below. Methods were prepared with guidance from industry standard documents.^{i,ii}

### Plasma Bioanalysis

A 10 mM DMSO stock is used to prepare spiking solutions of test compound in the range of 10-100,000 ng/mL in diluent (MeCN:water, 1;1). Calibration lines are prepared in control male Sprague-Dawley Rat plasma at known concentrations in the range of 1-10000 ng/mL by spiking 2.5 μL of calibration spiking solution into 25 μL control plasma. Experimental samples are thawed to RT and 25 μL aliquots are extracted alongside the calibration lines using protein precipitation (agitation for at least 5 min at RT with 400 μL of MeCN containing 25 ng/mL tolbutamide as an internal standard). Protein precipitates are separated from the extracted test compound by centrifugation at 4000 rpm for 5 min, 4° C. The resulting supernatants are diluted in a ratio of 1:2 with a relevant diluent (e.g. 0.1% formic acid in water or 1:1 MeOH:water). Samples are analysed by UPLC-MS/MS on either an API6500 QTrap or Waters 30 TQS mass spectrometer using previously optimised analytical MRM (multiple reaction monitoring) methods, specific to the test compound. The concentration of test compound in isolated samples is determined following analysis of the samples against the two replicates of the calibration line, injected before and after the sample set with an appropriate regression and weighting used. Only samples within 20% of the expected test concentration are included in the calibration line and any samples that fall outside of the limits of the calibration line will be deemed to be less than or above the limit of quantification (LLoQ/ALoQ).

### Brain Bioanalysis

A 10 mM DMSO stock is used to prepare spiking solutions of test compound in the range of 10-100,000 ng/mL in diluent (1:1 MeCN:water). Calibration lines are prepared in control male Sprague-Dawley Rat brain homogenate at known concentrations in the range of 3-30000 ng/g by spiking 2.5 μL of calibration spiking solution into 25 μL control homogenate. To prepare control and experimental brain homogenates, brains are thawed, weighed and a volume of diluent added (water) in the ratio of 2 mL per gram of brain. Homogenisation of brains is performed by bead-beater homogenisation using Precellys Evolution and CK14 7 mL small ceramic bead homogenisation tubes. Aliquots of 25 μL experimental sample are extracted alongside the calibration lines using protein precipitation (agitation for at least 5 min at RT with 400 μL of MeCN containing 25 ng/mL tolbutamide as an internal standard). Protein precipitates are separated from the extracted test compound by centrifugation at 4000 rpm for 5 min, 4° C. The resulting supernatants are diluted in a ratio of 1:2 with a relevant diluent (e.g. 0.1% formic acid in water or 1:1 MeOH:water). Samples are analysed by UPLC-MS/MS on either an API6500 QTrap or Waters TQS mass spectrometer using previously optimised analytical MRM (multiple reaction monitoring) methods, specific to the test compound. The concentration of test compound in isolated samples is determined following analysis of the samples against the two replicates of the calibration line, injected before and after the sample set with an appropriate regression and weighting used. Only samples within 20% of the expected test concentration are included in the calibration line and any samples that fall outside of the limits of the calibration line will be deemed to be less than or above the limit of quantification (LLoQ/ALoQ).

### Determination of Brain to Plasma (B:P) Ratio

Total brain to plasma (B:P) ratios were calculated by dividing the concentration in the brain by the concentration in plasma for each timepoint. The mean brain to plasma ratio is calculated by averaging these brain to plasma ratios across certain timepoints. Table 10 shows the brain to plasma (B:P) ratios for compounds of the invention and reference examples. The examples of the invention have much improved brain to plasma (B:P) ratios compared to the reference examples. Therefore, compounds of the invention are particularly useful in the treatment of certain diseases and conditions in which blood-brain barrier penetration is important. It is noted that blood-brain barrier penetration is unpredictable and is established empirically. Overcoming the challenges associated with delivering therapeutic agents to specific regions of the brain presents a major challenge to treatment of most brain disorders.

TABLE 10
Brain to plasma ratio in rat

| | | | | Mean | Timepoints |
|---|---|---|---|---|---|
| | | | Dose | B:P | averaged |
| Ex. | Formulation | Route | (mg/kg) | ratio | (h) |
| Ref. | 30% HPBC in water | i.v. | 1 | <0.01 | 0.17-1 |
| ex. 1 | | | | | |
| 13 | 30% HPBC in water | p.o. | 3.0 | 0.79 | 0.5-4 |
| 24 | 30% HPBC in water | p.o. | 3.0 | 0.68 | 0.5-4 |
| 35 | 30% HPBC in water | p.o. | 3.0 | 1.11 | 1 |
| 42 | 30% HPBC in water | p.o. | 3.0 | 0.98 | 0.5-4 |
| 47 | 30% HPBC in water | p.o. | 3.0 | 1.54 | 0.5-4 |
| 51 | 3.3% NMP in 30% HPBC | i.v. | 1.5 | 0.67 | 0.5-1 |
| | in water | | | | |
| 53 | 5% NMP in 30% HPBC in | p.o. | 3.0 | 0.82 | 0.5-1 |
| | water | | | | |
| 54 | 5% NMP in 30% HPBC in | i.v. | 0.5 | 0.53 | 0.17-1 |
| | water | | | | |
| 56 | 30% HPBC in water | p.o. | 3.0 | 1.91 | 0.5-4 |
| 61 | 30% HPBC in water | p.o. | 3.0 | 0.45 | 0.5-4 |
| 63 | 30% HPBC in water | p.o. | 3.0 | 0.64 | 0.5-4 |
| 65 | 30% HPBC in water | p.o. | 3.0 | 0.31 | 0.5-4 |
| 88 | 30% HPBC in water | P.o. | 3.0 | 0.69 | 0.5-4 |

### Determination of Bioavailability, Clearance and Half-Life In Vivo

Pharmacokinetic parameters such as bioavailability (% F), clearance (CL), half-life (T½) and volume of distribution are calculated by non-compartmental analysis using Phoenix Winnonlin 64 software (build 8.0). The bioavailability was calculated from p.o. dosed rats, whereas the clearance and half-life were calculated from i.v. dosed rats. Briefly, in vivo plasma concentrations, timepoints and dose values are imported into the software in a compatible format. Plasma concentration for each animal is plotted against time, and the elimination phase identified and selected. The area under the curve for each plot is calculated using a linear trapezoidal linear interpolation from which pharmacokinetic parameters can subsequently be determined. Table 11 shows pharmacokinetic parameters for compounds of the invention and reference examples. Compounds of the invention have much improved clearance (CL) and half-life (T½) compared to the reference examples.

TABLE 11
Rat PK profile

| | | i.v. | p.o. | | | |
|---|---|---|---|---|---|---|
| | | dose | dose | | CL | T½ |
| Ex. | Formulation | (mg/kg) | (mg/kg) | % F | (mL/min/kg) | (h) |
| Ref. | 30% HPBC in | 1 | 3 | 4 | 22 | 2.1 |
| ex. 1 | water | | | | | |
| Ref. | 20% PEG/24% | 1 | 3 | 113 | 12 | 1.2 |
| ex. 4 | HPBC in water | | | | | |
| 54 | 5% NMP, 30% | 0.5 | 3 | 97 | 4.0 | 7.6 |
| | HPBC in water | | | | | |
| 56 | 5% NMP, 30% | 1 | 3 | 97 | 1.9 | 20 |
| | HPBC in water | | | | | |
| 81 | 5% NMP, 30% | 1 | 3 | 102 | 3.0 | 6.2 |
| | HPBC in water | | | | | |

### Kinetic Aqueous Solubility Assay

Using a 10 mM stock solution of test compound in 100% DMSO, test compounds were spiked into 0.05M potassium phosphate buffer (pH 7.4) at a final concentration of 250 μM. Samples in buffer (n=2 aqueous samples, in 96-well plates) were allowed to equilibrate at RT on an orbital shaker for 30 min (300 rpm) to induce precipitation of test compounds. The appearance of each sample was determined by visual examination and noted (clear, cloudy, precipitate observed etc.). The aqueous phosphate buffer samples were filtered using a Multiscreen HTS solubility filter plate (Millipore) and filtrate was analysed by LC-UV alongside calibration standards of the test compounds prepared at 5, 25, 100 and 250 μM in 50:50 acetonitrile:water. The concentration of compound in phosphate buffer filtrate was determined by comparing the UV absorbance peak area of each replicate against that of the calibration standards. Table 12 shows kinetic aqueous solubility values for compounds of the invention and reference examples. Compounds of the invention have much improved solubility compared to the reference examples.

TABLE 12
Kinetic aqueous solubility

| Ex. | Solubility (μM) |
|---|---|
| Ref. ex. 2 | 9 |
| Ref. ex. 3 | 56 |
| Ref. ex. 4 | <5 |
| 2 | 215 |
| 3 | 140 |
| 4 | 180 |
| 12 | 140 |
| 16 | 180 |
| 18 | 200 |
| 20 | 100 |
| 25 | 170 |
| 27 | 100 |
| 30 | 140 |
| 42 | 120 |
| 51 | 190 |
| 52 | 160 |
| 53 | 170 |
| 54 | 170 |
| 55 | 150 |
| 57 | 170 |
| 58 | 180 |
| 59 | 190 |
| 60 | 210 |
| 61 | 160 |
| 63 | 120 |
| 65 | 170 |
| 71 | 110 |
| 78 | 150 |
| 82 | 170 |

hERG Assay

The human ether-a-go-go related gene (hERG) potassium channel (Kᵥ11.1) contributes to human cardiac action potential repolarisation. Inhibition of hERG channels can prolong the human cardiac action potential, resulting in QTc prolongation and potentially lethal arrhythmias (e.g. Torsade de Pointes).

Test samples were screened against the hERG channel on a QPatch 48 gigaseal automated patch clamp platform, using a Chinese Hamster Ovary (CHO) cell line stably expressing the human ether-á-go-go related gene, which encodes the hERG channel. All recordings were made in the conventional whole-cell configuration and performed at RT (−21° C.) using standard single hole chips (Rchip 1.5-4M0). Series resistance (4-15Mc) was compensated by >80%. Currents were elicited from a holding potential of −90 mV using the industry standard “⁺40/−40” voltage protocol, which was applied at a stimulus frequency of 0.1 Hz. On achieving the whole-cell configuration, vehicle (0.1% DMSO v/v in external recording solution) was applied to each cell in two bolus additions with a 2 min recording period between each addition. Following the vehicle period, eight increasing concentrations of test sample were applied from 0.003 μM to 10 μM as a single bolus addition per test concentration, and the effects on hERG tail current amplitude measured during the 2 min recording period. Each eight-point concentration-response curve was constructed using cumulative single sample additions of each concentration to the same cell. The positive control used in this study was verapamil hydrochloride (Tocris, Cat #0654, Batch #5A/61673) and prepared to a stock concentration of 10 mM in 100% DMSO and kept as frozen aliquots.

Data analysis: All cells passing the QC parameters (i.e. >200 pA outward current, membrane resistance and rundown) are selected as ‘passed QC’ using the QPatch software, which then calculates the mean peak current for the last three sweeps at the end of each concentration application period from the cursor positions defined. Percent inhibition is calculated for each test concentration application period as the reduction in mean cursor value (peak current or charge) relative to the cursor value measured at the end of the control (i.e. vehicle) period. The percent inhibition values from each cell are used to construct concentration-response curves employing a four parametric logistic fit with 0 and 100% inhibition levels fixed at very low and very high concentrations, respectively, and a free Hill slope factor. The IC₅₀ (50% inhibitory concentration) and Hill coefficient are then determined, but only data from cells with Hill slopes within 0.5>nH<2.0 are included. The IC₅₀ data reported below represents the mean (and S.D.) of at least three separate cells (N≥3). If a test sample failed to achieve >50% block at the top concentration it was deemed inactive, and assigned an arbitrary IC₅₀ value of ′>10 μM. Table 13 shows hERG IC₅₀ values for compounds of the invention and reference examples. Compounds of the invention show reduced hERG inhibitory activity compared to the reference examples.

TABLE 13
hERG IC₅₀ (μM)

| Ex. | hERG IC₅₀ (μM) |
|---|---|
| Ref. ex. 2 | 1.9 |
| Ref. ex. 3 | 2.5 |
| Ref. ex. 4 | 4.7 |
| 18 | >10 |
| 24 | >10 |
| 26 | >10 |
| 35 | >10 |
| 47 | >10 |
| 50 | >10 |
| 51 | >10 |
| 52 | >10 |
| 53 | >10 |
| 56 | >10 |
| 61 | >10 |
| 63 | >10 |
| 82 | >10 |

### Ames Assay

Examples 50, 52 and 54 were tested, in vitro, for their ability to induce mutations in 2 histidine dependent auxotrophic mutants of Salmonella typhimurium, strains TA98 and TA100. The mutation screen was conducted using the plate incorporation method and was performed in both the presence and absence of S-9 mix (a liver post-mitochondrial fraction derived from the livers of Aroclor 1254 treated rats). The bacteria were exposed to the test items dissolved in DMSO, which was also the negative Control. The test items were tested up to the regulatory maximum dose level. The dose levels used were 5, 15, 50, 150, 500, 1500 or 5000 μg/plate, unless the highest treatment level of the test items was limited by solubility, or toxicity against the background bacterial lawn. Dose levels were expressed in terms of the free base.

There were no increases of 2-fold or greater in revertant numbers compared with negative Control values in either strain at any dose level in the presence or absence of S-9 mix, so Examples 50, 52 and 54 were not mutagenic in the Bacterial Reverse Mutation (i.e. Ames) Test under the conditions of this screen.

### Off-Target Selectivity Assays

Examples 50 and 54 were tested for kinase selectivity against the Eurofins KinaseProfiler™ kinase screen consisting of 430 wild-type and mutant kinases, including a panel of lipid kinases, up to a top concentration of 1 μM. The radiometric kinase activity assays were run at ATP Km. Neither of the compounds showed >50% inhibition against any of the kinases at the top concentration.

Example 50 was screened against the Eurofins SafetyScreen™ panel of 87 targets across GPCRs, ion channels, and enzymes. Only 2 of 87 targets were inhibited >50% at a top concentration of 10 μM, with a maximum inhibition of 65%.

### REFERENCES

ⁱ Food and Drug Administration. (2018). Bioanalytical Method Validation: Guidance for Industry.
ⁱⁱ Whitmire M, Ammerman J, de Lisio P, Killmer J, Kyle D (2011) LCMS/MS Bioanalysis Method Development, Validation, and Sample Analysis: Points to Consider When Conducting Nonclinical and Clinical Studies in Accordance with Current Regulatory Guidances. J Anal Bioanal Techniques S4:001. doi:10.4172/2155-9872.S4-001.

1. A compound of formula (I): 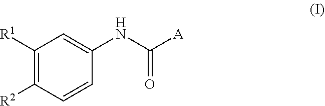
or a pharmaceutically acceptable salt, solvate, hydrate, geometrical isomer, tautomer, optical isomer, N-oxide thereof, and/or prodrug thereof, wherein
R¹ is selected from the group consisting of H and halo, preferably H, F, and Cl, more preferably H;
R² is selected from the group consisting of —OCF₂Cl, —OCF₃, —SCF₃, —SCF₂Cl, —CF₂CF₃, —CF₂CF₂Cl, —OCF₂CF₃, —SF₅, OF₂CH₃, —SOCF₃, —SO₂CF₃, —OCF₂CF₂H, and —SCF₂H, preferably —OCF₂C₁ and —OCF₃;
A is 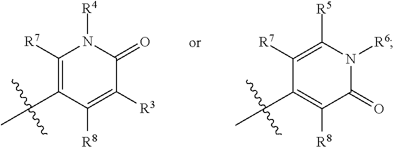
R³, R⁴, R⁵, and R⁶ are independently selected from the group consisting of:
(i) H, halo, —OH, —C(O)NR^{d}R^{e}, —NR^{a}R^{b}, cyano, —C(O)OR^{c}, and —C(O)R^{c};
(ii) C₁-C₇ alkyl, C₂-C₆ alkenyl, C₂-C₆ alkynyl, C₁-C₆ alkoxy, optionally substituted with one or more substituents independently selected from the group consisting of —NR^{a}R^{b}, cyano, —OR^{c}, halo, oxo, 6- to 10-membered aryl, 5- to 10-membered heteroaryl, and 4- to 10-membered heterocycle, optionally wherein the 5- to 10-membered heteroaryl and 4- to 10-membered heterocycle are independently substituted with one or more substituents selected from halo and C₁-C₇ alkyl, wherein the C₁-C₇ alkyl is optionally substituted with one or more halo atoms;
(iii) 6- to 10-membered aryl and 5- to 10 membered heteroaryl, optionally substituted with one or more substituents independently selected from the group consisting of halo, —C(O)NR^{d}R^{e}, —NR^{d}R^{e}, —OH, oxo, cyano, —C(O)OR^{c}, and —C(O)R^{c}, C₁-C₇ alkyl, C₂-C₆ alkenyl, C₂-C₆ alkynyl, C₁-C₆ alkoxy, (C₁-C₃ alkyl)-O—(C₁-C₃ alkyl), and 4- to 10-membered heterocycle, wherein the 4- to 10-membered heterocycle is optionally substituted with an oxo group, wherein the alkyl, alkenyl, and alkynyl groups are each optionally independently substituted with one or more halo atoms; and
(iv) 4- to 10-membered heterocycle, optionally substituted with one or more substituents independently selected from the group consisting of halo, oxo, —C(O)NR^{d}R^{e}, —NR^{d}R^{e}, cyano, —C(O)OR^{c}, and —C(O)R^{c}, C₁-C₇ alkyl, C₂-C₆ alkenyl, C₂-C₆ alkynyl, C₁-C₆ alkoxy, (C₁-C₃ alkyl)-O—(C₁-C₃ alkyl), 6- to 10-membered aryl, 5- to 10-membered heteroaryl, and 4- to 10-membered heterocycle, wherein the alkyl, alkenyl, and alkynyl groups are each optionally independently substituted with one or more halo atoms;
each R⁷ and R⁸ is independently selected from the group consisting of H and halo, preferably H, F and Cl, more preferably H and F, most preferably H;
each R^{a}, R^{b}, and R^{c} is independently selected from H and C₁-C₇ alkyl, wherein the C₁-C₇alkyl is optionally substituted with one or more halo atoms; and
each R^{d} and R^{e} are independently selected from H and C₁-C₇ alkyl, wherein the C₁-C₇ alkyl is optionally substituted with one or more halo atoms, or R^{d} and R^{e} can be taken together with the nitrogen atom to which they are attached to form a 5- or 6-membered saturated, partially saturated, or unsaturated ring, wherein the ring contains one or more heteroatoms;
preferably R⁵ and R³ and/or R⁴ are selected from substituents (iii), more preferably R⁵ or R⁴ is selected from substituents (iii).

2. A compound according to claim 1, wherein the compound is a compound of formula (II): 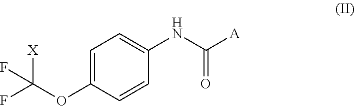
wherein X is F or C₁; and
wherein A is

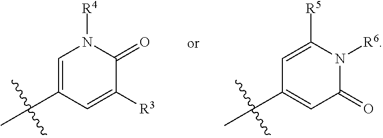

3. A compound according to claim 2, wherein X═Cl.

4. A compound according to any preceding claim, wherein R³, R⁴, R⁵ and R⁶ are independently selected from the group consisting of:
(i) H and cyano;
(ii) C₁-C₆ alkyl and C₁-C₆ alkoxy, optionally substituted with one or more substituents independently selected from halo and 5- or 6-membered heterocycle;
(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and
(iv) phenyl optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;
(v) 5- to 10-membered heteroaryl optionally substituted with one or more substituents independently selected from the group consisting of halo, cyano, oxo, C₁-C₆ alkyl, C₁-C₆ alkoxy, and (C₁-C₃ alkyl)-O—(C₁-C₃ alkyl), wherein the alkyl groups are optionally substituted with one or more halo atoms;
preferably R⁵ and R³ and/or R⁴ are selected from substituents (iv) and (v), more preferably R⁵ or R⁴ is selected from substituents (iv) and (v).

5. A compound according to any of claims 2 to 4, wherein the compound is a compound of formula (IIa): 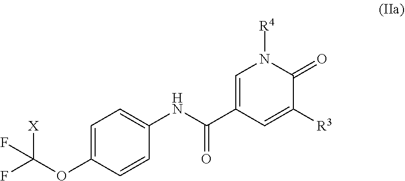
wherein R³ and R⁴ are independently selected from the group consisting of:
(i) H and cyano;
(ii) C₁-C₆ alkyl and C₁-C₆ alkoxy, optionally substituted with one or more substituents independently selected from halo and 5- or 6-membered heterocycle;
(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and
(iv) phenyl, optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;
(v) 5- to 10-membered heteroaryl optionally substituted with one or more substituents independently selected from the group consisting of halo, cyano, oxo, C₁-C₆ alkyl, C₁-C₆ alkoxy, and (C₁-C₃ alkyl)-O—(C₁-C₃ alkyl), wherein the alkyl groups are optionally substituted with one or more halo atoms;
preferably R³ and/or R⁴ is selected from substituents (iv) and (v), more preferably R⁴ is selected from substituents (iv) and (v).

6. A compound according to claim 5, wherein R³ is selected from the group consisting of:
(i) H, C₁-C₆ alkoxy, and cyano;
(ii) C₁-C₆ alkyl, optionally substituted with one or more halo atoms;
(iii) phenyl and 5- or 6-membered heteroaryl, optionally substituted with one or more substituents independently selected from the group consisting of halo and C₁-C₆ alkyl, wherein the C₁-C₆ alkyl is optionally substituted with one or more halo atoms; and
(iv) 5- or 6-membered heterocycle, optionally substituted with one or more substituents independently selected from the group consisting of halo and C₁-C₆ alkyl, wherein the C₁-C₆ alkyl is optionally substituted with one or more halo atoms.
preferably R³ is selected from substituents (iii).

7. A compound according to claim 5 or 6, wherein R⁴ is selected from the group consisting of:
(i) H;
(ii) C₁-C₆ alkyl, wherein the C₁-C₆ alkyl is optionally substituted with one or more substituents selected from halo and 5- or 6-membered heterocycle;
(iii) phenyl, optionally substituted with one or more substituents independently selected from halo, cyano, C₁-C₆ alkyl, C₁-C₆ alkoxy, and 4-membered heterocycle, wherein the 4-membered heterocycle is optionally substituted with an oxo group, wherein the C₁-C₆ alkyl is optionally substituted with one or more halo atoms;
(iv) 5- or 6-membered heteroaryl, optionally substituted with one or more substituents independently selected from halo, cyano, C₁-C₆ alkyl, C₁-C₆ alkoxy, and (C₁-C₃ alkyl)-O—(C₁-C₃ alkyl), wherein the alkyl groups are optionally substituted with one or more halo atoms;
(v) 5- or 6-membered heterocycle, optionally substituted with one or more substituents independently selected from the group consisting of halo, and C₁-C₆ alkyl, wherein the C₁-C₆ alkyl is optionally substituted with one or more halo atoms;
(vi) group B 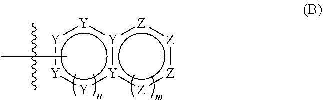
wherein each Y and Z is independently selected from C, S, O, and N, at least one Y or Z is S, O or N,
each Y and Z is optionally independently substituted with halo, or C₁-C₆ alkyl, wherein the C₁-C₆ alkyl is optionally substituted with one or more halo atoms,
n is 0 or 1,
m is 0 or 1;
(vii) group C 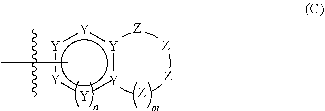
wherein each Y and Z is independently selected from C, S, O, and N,
at least one Y or Z is S, O or N,
each Y and Z is optionally independently substituted with halo or C₁-C₆ alkyl, wherein the C₁-C₆alkyl is optionally substituted with one or more halo atoms,
n is 0 or 1,
m is 0 or 1; and
(viii) group D 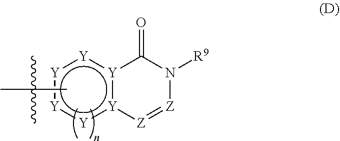
wherein each Y and Z is independently selected from C, S, O, and N, at least one Z is C,
each Y and Z is optionally independently substituted with halo or C₁-C₆ alkyl, wherein the C₁-C₆alkyl is optionally substituted with one or more halo atoms, R⁹ is selected from halo and C₁-C₆ alkyl, wherein the C₁-C₆ alkyl is optionally substituted with one or more halo atoms,
n is 0 or 1;
preferably R⁴ is selected from substituents (iii), (iv), (vi), (vii), and (viii).

8. A compound according to any of claims 2 to 4, wherein the compound is a compound of formula (IIb): 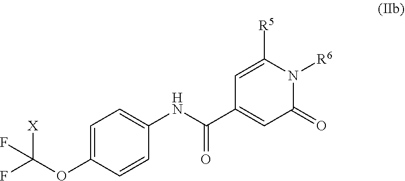
wherein R⁵ and R⁶ are independently selected from the group consisting of:
(i) H and cyano;
(ii) C₁-C₆ alkyl and C₁-C₆ alkoxy, optionally substituted with one or more substituents independently selected from halo and 5- or 6-membered heterocycle; and
(iii) 5- or 6-membered heterocycle, optionally substituted with one or more substituents independently selected from the group consisting of halo, and C₁-C₆ alkyl, wherein the C₁-C₆ alkyl is optionally substituted with one or more halo atoms; and
(iv) phenyl, optionally substituted with one or more substituents independently selected from the group consisting of halo, cyano, C₁-C₆ alkyl, C₁-C₆ alkoxy, and 4-membered heterocycle, wherein the 4-membered heterocycle is optionally substituted with an oxo group, wherein the alkyl groups are optionally substituted with one or more halo atoms;
(v) 5- to 10-membered heteroaryl optionally substituted with one or more substituents independently selected from the group consisting of halo, cyano, oxo, C₁-C₆ alkyl, C₁-C₆ alkoxy, (C₁-C₃ alkyl)-O—(C₁-C₃ alkyl), wherein the alkyl groups are optionally substituted with one or more halo atoms;
preferably R⁵ is selected from substituents (iv) and (v).

9. A compound according to claim 8, wherein R⁵ is a 5- or 6-membered heteroaryl group, optionally substituted with one or more substituents independently selected from the group consisting of halo and C₁-C₆ alkyl, wherein the C₁-C₆ alkyl is optionally substituted with one or more halo atoms.

10. A compound according to claim 8 or 9, wherein R⁶ is selected from the group consisting of H and C₁-C₆ alkyl, wherein the C₁-C₆ alkyl is optionally substituted with one or more halo atoms.

11. A compound according to any preceding claim, wherein the compound is
N-[4-[Chloro(difluoro)methoxy]phenyl]-1-(3-ethoxy-1-methyl-pyrazol-4-yl)-6-oxo-pyridine-3-carboxamide;
1-(2-Morpholinoethyl)-6-oxo-N-[4-(trifluoromethoxy)phenyl]pyridine-3-carboxamide;
N-[4-(Chlorodifluoromethoxy)phenyl]-1-(1-methyl-1H-pyrazol-3-yl)-6-oxo-1,6-dihydropyridine-3-carboxamide;
N-[4-[Chloro(difluoro)methoxy]phenyl]-6-oxo-1-[(3S)-tetrahydrofuran-3-yl]pyridine-3-carboxamide;
N-[4-[Chloro(difluoro)methoxy]phenyl]-6-oxo-1-(1H-pyrazol-5-yl)pyridine-3-carboxamide;
N-[4-[Chloro(difluoro)methoxy]phenyl]-6-oxo-1-[(3R)-tetrahydrofuran-3-yl]pyridine-3-carboxamide;
N-[4-(Chlorodifluoromethoxy)phenyl]-1-[1-(difluoromethyl)-1H-pyrazol-3-yl]-6-oxo-1,6-dihydropyridine-3-carboxamide;
N-[4-[Chloro(difluoro)methoxy]phenyl]-6-oxo-1-thiazol-2-yl-pyridine-3-carboxamide;
N-[4-[Chloro(difluoro)methoxy]phenyl]-1-(2-methyl-3-pyridyl)-6-oxo-pyridine-3-carboxamide;
N-[4-[Chloro(difluoro)methoxy]phenyl]-1-(2,4-dimethoxypyrimidin-5-yl)-6-oxo-pyridine-3-carboxamide;
N-[4-[Chloro(difluoro)methoxy]phenyl]-6-oxo-1-thiazol-5-yl-pyridine-3-carboxamide;
N-[4-[Chloro(difluoro)methoxy]phenyl]-1-(2-methoxypyrimidin-5-yl)-6-oxo-pyridine-3-carboxamide;
N-[4-[Chloro(difluoro)methoxy]phenyl]-1-(6-methoxy-3-pyridyl)-6-oxo-pyridine-3-carboxamide;
N-[4-[Chloro(difluoro)methoxy]phenyl]-1-(1-isopropylpyrazol-4-yl)-6-oxo-pyridine-3-carboxamide;
N-[4-[Chloro(difluoro)methoxy]phenyl]-1-(1-cyclopropylpyrazol-4-yl)-6-oxo-pyridine-3-carboxamide;
N-[4-[Chloro(difluoro)methoxy]phenyl]-1-[(3R)-1-methylpyrrolidin-3-yl]-6-oxo-pyridine-3-carboxamide;
N-[4-[Chloro(difluoro)methoxy]phenyl]-1-[(3S)-1-methylpyrrolidin-3-yl]-6-oxo-pyridine-3-carboxamide;
N-[4-[Chloro(difluoro)methoxy]phenyl]-1-(1,5-dimethylpyrazol-4-yl)-6-oxo-pyridine-3-carboxamide;
N-[4-[Chloro(difluoro)methoxy]phenyl]-1-(3-cyclopropyl-1-methyl-pyrazol-4-yl)-6-oxo-pyridine-3-carboxamide;
N-[4-(Chlorodifluoromethoxy)phenyl]-1-(3-cyanophenyl)-6-oxo-1,6-dihydropyridine-3-carboxamide;
N-[4-[Chloro(difluoro)methoxy]phenyl]-1-[1-(cyclopropylmethyl)pyrazol-4-yl]-6-oxo-pyridine-3-carboxamide;
N-[4-[Chloro(difluoro)methoxy]phenyl]-1-[1-(2-methoxyethyl)pyrazol-4-yl]-6-oxo-pyridine-3-carboxamide;
1-(1-tert-Butylpyrazol-4-yl)-N-[4-[chloro(difluoro)methoxy]phenyl]-6-oxo-pyridine-3-carboxamide;
N-[4-(Chlorodifluoromethoxy)phenyl]-1-(3,4-difluorophenyl)-6-oxo-1,6-dihydropyridine-3-carboxamide;
N-[4-(Chlorodifluoromethoxy)phenyl]-1-(1-methyl-1H-pyrazol-5-yl)-6-oxo-1,6-dihydropyridine-3-carboxamide;
1-Methyl-6-oxo-N-[4-(trifluoromethoxy)phenyl]pyridine-3-carboxamide N-[4-(Chlorodifluoromethoxy)phenyl]-1-[1-(difluoromethyl)-1H-pyrazol-4-yl]-6-oxo-1,6-dihydropyridine-3-carboxamide;
N-[4-(Chlorodifluoromethoxy)phenyl]-6-oxo-1-(quinoxalin-5-yl)-1,6-dihydropyridine-3-carboxamide;
N-[4-(Chlorodifluoromethoxy)phenyl]-1-(4-methoxyphenyl)-6-oxo-1,6-dihydropyridine-3-carboxamide;
1-(5-Chloro-1-methyl-1H-pyrazol-4-yl)-N-[4-(chlorodifluoromethoxy)phenyl]-6-oxo-1,6-dihydropyridine-3-carboxamide;
N-[4-(Chlorodifluoromethoxy)phenyl]-1-(2,3-difluorophenyl)-6-oxo-1,6-dihydropyridine-3-carboxamide;
N-[4-(Chlorodifluoromethoxy)phenyl]-1-(2,3-dimethoxyphenyl)-6-oxo-1,6-dihydropyridine-3-carboxamide;
N-[4-(Chlorodifluoromethoxy)phenyl]-1-(3,4-dihydro-1H-2-benzopyran-6-yl)-6-oxo-1,6-dihydropyridine-3-carboxamide;
N-[4-(Chlorodifluoromethoxy)phenyl]-1-(1,3-dihydro-2-benzofuran-5-yl)-6-oxo-1,6-dihydropyridine-3-carboxamide;
N-[4-(Chlorodifluoromethoxy)phenyl]-1-(3-methoxyphenyl)-6-oxo-1,6-dihydropyridine-3-carboxamide;
N-[4-(Chlorodifluoromethoxy)phenyl]-1-(2,3-dihydro-1,4-benzodioxin-6-yl)-6-oxo-1,6-dihydropyridine-3-carboxamide;
N-[4-(Chlorodifluoromethoxy)phenyl]-1-(2-fluorophenyl)-6-oxo-1,6-dihydropyridine-3-carboxamide;
N-[4-(Chlorodifluoromethoxy)phenyl]-6-oxo-1-(quinoxalin-6-yl)-1,6-dihydropyridine-3-carboxamide;
N-[4-(Chlorodifluoromethoxy)phenyl]-1-(3,4-dihydro-1H-2-benzopyran-7-yl)-6-oxo-1,6-dihydropyridine-3-carboxamide;
N-[4-(Chlorodifluoromethoxy)phenyl]-1-(3,4-dihydro-1H-2-benzopyran-8-yl)-6-oxo-1,6-dihydropyridine-3-carboxamide;
N-[4-(Chlorodifluoromethoxy)phenyl]-1-(3,4-dihydro-1H-2-benzopyran-5-yl)-6-oxo-1,6-dihydropyridine-3-carboxamide;
N-[4-(Chlorodifluoromethoxy)phenyl]-1-(4-fluorophenyl)-6-oxo-1,6-dihydropyridine-3-carboxamide;
N-[4-(Chlorodifluoromethoxy)phenyl]-1-(3,4-dimethoxyphenyl)-6-oxo-1,6-dihydropyridine-3-carboxamide;
N-[4-(Chlorodifluoromethoxy)phenyl]-1-(1-methyl-1H-1,2,3-benzotriazol-5-yl)-6-oxo-1,6-dihydropyridine-3-carboxamide;
1-(3-Chloro-1-methyl-1H-pyrazol-4-yl)-N-[4-(chlorodifluoromethoxy)phenyl]-6-oxo-1,6-dihydropyridine-3-carboxamide;
N-[4-(Chlorodifluoromethoxy)phenyl]-1-(3,5-difluorophenyl)-6-oxo-1,6-dihydropyridine-3-carboxamide;
N-[4-(Chlorodifluoromethoxy)phenyl]-1-(3-fluorophenyl)-6-oxo-1,6-dihydropyridine-3-carboxamide;
1-(1,3-Benzoxazol-4-yl)-N-[4-(chlorodifluoromethoxy)phenyl]-6-oxo-1,6-dihydropyridine-3-carboxamide;
N-[4-[Chloro(difluoro)methoxy]phenyl]-6-oxo-1-[4-(2-oxoazetidin-1-yl)phenyl]pyridine-3-carboxamide;
6-Oxo-1-(pyrimidin-5-yl)-N-[4-(trifluoromethoxy)phenyl]-1.6-dihydropyridine-3-carboxamide;
N-[4-(Chlorodifluoromethoxy)phenyl]-6-oxo-1-(pyrimidin-5-yl)-1,6-dihydropyridine-3-carboxamide;
N-[4-[Chloro(difluoro)methoxy]phenyl]-1-(1-methylpyrazol-4-yl)-6-oxo-pyridine-3-carboxamide;
N-[4-[Chloro(difluoro)methoxy]phenyl]-6-oxo-1-(3-pyridyl)pyridine-3-carboxamide;
N-[4-[Chloro(difluoro)methoxy]phenyl]-1-(5-fluoro-3-pyridyl)-6-oxo-pyridine-3-carboxamide;
N-[4-[Chloro(difluoro)methoxy]phenyl]-1-(5-cyano-3-pyridyl)-6-oxo-pyridine-3-carboxamide;
N-[4-[Chloro(difluoro)methoxy]phenyl]-6-oxo-1-phenyl-pyridine-3-carboxamide;
N-[4-[Chloro(difluoro)methoxy]phenyl]-1-(5-methyl-3-pyridyl)-6-oxo-pyridine-3-carboxamide;
N-[4-[Chloro(difluoro)methoxy]phenyl]-5-methoxy-6-oxo-1-pyrimidin-5-yl-pyridine-3-carboxamide;
N-[4-[Chloro(difluoro)methoxy]phenyl]-5-methyl-6-oxo-1-pyrimidin-5-yl-pyridine-3-carboxamide;
N-[4-(Chlorodifluoromethoxy)phenyl]-5-(morpholin-4-yl)-6-oxo-1-(pyrimidin-5-yl)-1,6-dihydropyridine-3-carboxamide;
N-[4-[Chloro(difluoro)methoxy]phenyl]-1-(5-methoxy-3-pyridyl)-6-oxo-pyridine-3-carboxamide;
N-[4-[Chloro(difluoro)methoxy]phenyl]-6-oxo-1-[5-(trifluoromethyl)-3-pyridyl]pyridine-3-carboxamide;
N-[4-[Chloro(difluoro)methoxy]phenyl]-1-(5-chloro-3-pyridyl)-6-oxo-pyridine-3-carboxamide;
N-[4-(Chlorodifluoromethoxy)phenyl]-1-{imidazo[1,2-b]pyridazin-3-yl}-6-oxo-1,6-dihydropyridine-3-carboxamide;
N-[4-(Chlorodifluoromethoxy)phenyl]-6′-methyl-2-oxo-2H-[1,3′-bipyridine]-5-carboxamide;
N-[4-(Chlorodifluoromethoxy)phenyl]-5′-(difluoromethyl)-2-oxo-2H-[1,3′-bipyridine]-5-carboxamide;
N-[4-(Chlorodifluoromethoxy)phenyl]-1-(1,5-naphthyridin-3-yl)-6-oxo-1,6-dihydropyridine-3-carboxamide;
N-[4-(Chlorodifluoromethoxy)phenyl]-1-(3-methyl-4-oxo-3,4-dihydroquinazolin-7-yl)-6-oxo-1,6-dihydropyridine-3-carboxamide;
N-[4-(Chlorodifluoromethoxy)phenyl]-1-{2H,3H-[1,4]dioxino[2,3-b]pyridin-7-yl}-6-oxo-1,6-dihydropyridine-3-carboxamide;
N-[4-(Chlorodifluoromethoxy)phenyl]-1-{1-methyl-1H-pyrazolo[4,3-b]pyridin-6-yl}-6-oxo-1,6-dihydropyridine-3-carboxamide;
N-[4-(Chlorodifluoromethoxy)phenyl]-1-{2-methyl-2H-pyrazolo[4,3-b]pyridin-6-yl}-6-oxo-1,6-dihydropyridine-3-carboxamide;
6′-Chloro-N-[4-(chlorodifluoromethoxy)phenyl]-2-oxo-2H-[1,3′-bipyridine]-5-carboxamide;
N-[4-(Chlorodifluoromethoxy)phenyl]-1-{2H,3H-furo[2,3-b]pyridin-5-yl}-6-oxo-1,6-dihydropyridine-3-carboxamide;
2′-Chloro-N-[4-(chlorodifluoromethoxy)phenyl]-2-oxo-2H-[1,3′-bipyridine]-5-carboxamide;
N-[4-(Chlorodifluoromethoxy)phenyl]-2-oxo-6′-(trifluoromethyl)-2H-[1.3′-bipyridine]-5-carboxamide;
N-[4-(Chlorodifluoromethoxy)phenyl]-1-methyl-6-oxo-1,6-dihydropyridine-3-carboxamide;
N-[4-(Chlorodifluoromethoxy)phenyl]-1-(oxan-4-yl)-6-oxo-1,6-dihydropyridine-3-carboxamide;
5-{[4-(Chlorodifluoromethoxy)phenyl]carbamoyl}-2-oxo-2H-[1.3′-bipyridin]-1′-ium-t-olate;
N-[4-(Chlorodifluoromethoxy)phenyl]-5-cyano-1-(1-methyl-1H-pyrazol-4-yl)-6-oxo-1.6-dihydropyridine-3-carboxamide;
N-[4-(Chlorodifluoromethoxy)phenyl]-6-oxo-5-(1H-pyrazol-3-yl)-1,6-dihydropyridine-3-carboxamide;
N-[4-[Chloro(difluoro)methoxy]phenyl]-1-methyl-6-oxo-5-(3-pyridyl)pyridine-3-carboxamide;
N-[4-(Chlorodifluoromethoxy)phenyl]-1-methyl-6-oxo-5-(pyrimidin-5-yl)-1,6-dihydropyridine-3-carboxamide;
N-[4-(Chlorodifluoromethoxy)phenyl]-1-methyl-5-(1-methyl-1H-pyrazol-5-yl)-6-oxo-1,6-dihydropyridine-3-carboxamide;
N-[4-(Chlorodifluoromethoxy)phenyl]-5-(1-ethyl-1H-pyrazol-3-yl)-1-methyl-6-oxo-1,6-dihydropyridine-3-carboxamide;
N-[4-(Chlorodifluoromethoxy)phenyl]-1-methyl-5-(1-methyl-1H-pyrazol-3-yl)-6-oxo-1,6-dihydropyridine-3-carboxamide;
N-[4-[Chloro(difluoro)methoxy]phenyl]-1-methyl-5-(1-methylpyrazol-4-yl)-6-oxo-pyridine-3-carboxamide;
N-[4-[Chloro(difluoro)methoxy]phenyl]-5-(5-fluoro-3-pyridyl)-1-methyl-6-oxo-pyridine-3-carboxamide;
N-[4-[Chloro(difluoro)methoxy]phenyl]-1-methyl-6-oxo-5-phenyl-pyridine-3-carboxamide;
N-[4-(Chlorodifluoromethoxy)phenyl]-1-(1-methyl-1H-pyrazol-4-yl)-2-oxo-1,2-dihydro-[3,3′-bipyridine]-5-carboxamide;
N-[4-[Chloro(difluoro)methoxy]phenyl]-1-methyl-6-oxo-5-pyrazol-1-yl-pyridine-3-carboxamide;
N-[4-[Chloro(difluoro)methoxy]phenyl]-5-imidazol-1-yl-1-methyl-6-oxo-pyridine-3-carboxamide;
N-[4-(Chlorodifluoromethoxy)phenyl]-2-oxo-6-(1H-pyrazol-5-yl)-1,2-dihydropyridine-4-carboxamide;
2-Oxo-6-(pyrimidin-5-yl)-N-[4-(trifluoromethoxy)phenyl]-1,2-dihydropyridine-4-carboxamide; or
N-[4-(Chlorodifluoromethoxy)phenyl]-1-methyl-6-(1-methyl-1H-pyrazol-4-yl)-2-oxo-1,2-dihydropyridine-4-carboxamide;
or a pharmaceutically acceptable salt, solvate, hydrate, geometrical isomer, tautomer, optical isomer, N-oxide thereof, and/or prodrug thereof.

12. A pharmaceutical composition comprising a compound according to any preceding claim and a pharmaceutically acceptable carrier, excipient, and/or diluent.

13. The compound according to any one of claims 1 to 11, or the pharmaceutical composition of claim 12, for use in therapy.

14. The compound according to any one of claims 1 to 11, or the pharmaceutical composition of claim 12, for use in the treatment or prevention of a neurodegenerative disorder, a cancer, a prion disease, a viral infection, diabetes, an inflammatory disease, acute pancreatitis (preferably severe acute pancreatitis), pulmonary arterial hypertension, or a skeletal or muscular dystrophy, preferably a neurodegenerative disorder or a cancer.

15. Use of the compound according to any one of claims 1 to 11 for the manufacture of a medicament for the treatment or prevention of a neurodegenerative disorder, a cancer, a prion disease, a viral infection, diabetes, an inflammatory disease, acute pancreatitis (preferably severe acute pancreatitis), pulmonary arterial hypertension, or a skeletal or muscular dystrophy, preferably a neurodegenerative disorder or a cancer.

16. A method for the treatment or prevention of a disease or condition responsive to c-Abl inhibition comprising administering a therapeutically effective amount of the compound according to any one of claims 1 to 11, or the pharmaceutical composition of claim 12, to a subject.

17. The method of claim 16, wherein the disease or condition is a neurodegenerative disorder, a cancer, a prion disease, a viral infection, diabetes, an inflammatory disease, acute pancreatitis (preferably severe acute pancreatitis), pulmonary arterial hypertension, or a skeletal or muscular dystrophy, preferably a neurodegenerative disorder or a cancer.

18. The compound or pharmaceutical composition for use according to claim 14, the use of the compound according to claim 15, or the method of claim 16, wherein the neurodegenerative disorder is selected from Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, and spinocerebellar ataxia, fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

19. The compound for use, use of the compound, or method, of claim 18, wherein the neurodegenerative disorder is amyotrophic lateral sclerosis (ALS) or Parkinson's disease, preferably ALS.

20. The compound or pharmaceutical composition for use according to claim 14, the use of the compound according to claim 15, or the method of claim 16, wherein the cancer is leukaemia, preferably chronic myeloid leukaemia (CML), acute lymphoblastic leukaemia (ALL), acute myelogenous leukaemia (AML), or mixed-phenotype acute leukaemia (MPAL), or any central nervous system (CNS) metastases thereof, preferably CML or ALL.
